(19) United States
(12) Patent Application Publication
(10) Pub. No.: US 2011/0053173 A1
(43) Pub. Date: Mar. 3, 2011

(54) Game with detection capability

(75) Inventors: Leroy E. Hood, Seattle, WA (US); Roderick A. Hyde, Redmond, WA (US); Edward K.Y. Jung, Bellevue, WA (US); Eric C. Leuthardt, St Louis, MO (US); Elizabeth A. Sweeney, Seattle, WA (US); Clarence T. Tegreene, Bellevue, WA (US); Lowell L. Wood, JR., Bellevue, WA (US)

(73) Assignee: Searete LLC, a limited liability corporation of the State of Delaware

(21) Appl. No.: 12/657,166

(22) Filed: Jan. 13, 2010

Related U.S. Application Data
(63) Continuation-in-part of application No. 12/584,055, filed on Aug. 28, 2009.
Application No. 12/584,055 is a continuation-in-part of application No. 12/584,054, filed on Aug. 28, 2009.
Application No. 12/584,054 is a continuation-in-part of application No. 12/584,364, filed on Sep. 2, 2009.

(51) Int. Cl. G01N 33/53; A63F 9/24; G01N 33/566

(52) U.S. Cl. 435/7.1; 463/40; 436/501

(57) ABSTRACT

Methods and systems are described herein relating to game systems. In one aspect, a game system includes: at least one game component configured for use in a game system by an individual player; at least one sensor system operably connected to the at least one game component and configured to detect one or more analyte, the at least one sensor system including a signal transmitter; at least one signal detector configured to detect a signal transmitted from the at least one sensor system; and at least one principal game unit operably connected to the at least one signal detector, the at least one principal game unit including at least one signal transmitter configured to transmit a signal responsive to the at least one signal detector.

### CROSS-REFERENCE TO RELATED APPLICATIONS

The present application is related to and claims the benefit of the earliest available effective filing date(s) from the following listed application(s) (the “Related Applications”) (e.g., claims earliest available priority dates for other than provisional patent applications or claims benefits under 35 USC §119(e) for provisional patent applications, for any and all parent, grandparent, great-grandparent, etc. applications of the Related Application(s)). All subject matter of the Related Applications and of any and all parent, grandparent, great-grandparent, etc. applications of the Related Applications is incorporated herein by reference to the extent such subject matter is not inconsistent herewith.

### RELATED APPLICATIONS

For purposes of the USPTO extra-statutory requirements, the present application constitutes a continuation-in-part of U.S. patent application Ser. No. 12/584,055, entitled DEVICES AND METHODS FOR DETECTING AN ANALYTE 1N SALIVARY FLUID, naming Leroy E. Hood, Edward K. Y. Jung, Elizabeth A. Sweeney, Clarence T. Tegreene, and Lowell L. Wood, Jr. as inventors, filed Aug. 28, 2009, which is currently co-pending, or is an application of which a currently co-pending application is entitled to the benefit of the filing date.
For purposes of the USPTO extra-statutory requirements, the present application constitutes a continuation-in-part of United States Patent application Ser. No. 12/584,054, entitled BEVERAGE IMMERSATE WITH DETECTION CAPABILITY, naming Leroy E. Hood, Edward K. Y. Jung, Elizabeth A. Sweeney, Clarence T. Tegreene, and Lowell L. Wood, Jr. as inventors, filed Aug. 28, 2009, which is currently co-pending, or is an application of which a currently co-pending application is entitled to the benefit of the filing date.
For purposes of the USPTO extra-statutory requirements, the present application constitutes a continuation-in-part of U.S. patent application Ser. No. 12/584,364, entitled BEVERAGE CONTAINERS WITH DETECTION CAPABILITY, naming Leroy E. Hood, Edward K. Y. Jung, Elizabeth A. Sweeney, Clarence T. Tegreene, and Lowell L. Wood, Jr. as inventors, filed Sep. 2, 2009, which is currently co-pending, or is an application of which a currently co-pending application is entitled to the benefit of the filing date.

The United States Patent Office (USPTO) has published a notice to the effect that the USPTO's computer programs require that patent applicants reference both a serial number and indicate whether an application is a continuation or continuation-in-part. Stephen G. Kunin, Benefit of Prior-Filed application, USPTO Official Gazette Mar. 18, 2003, available at http://www.uspto.gov/web/offices/com/sol/og/2003/week11/patbene.htm. The present Applicant Entity (hereinafter “Applicant”) has provided above a specific reference to the application(s) from which priority is being claimed as recited by statute. Applicant understands that the statute is unambiguous in its specific reference language and does not require either a serial number or any characterization, such as “continuation” or “continuation-in-part,” for claiming priority to U.S. patent applications. Notwithstanding the foregoing, Applicant understands that the USPTO's computer programs have certain data entry requirements, and hence Applicant is designating the present application as a continuation-in-part of its parent applications as set forth above, but expressly points out that such designations are not to be construed in any way as any type of commentary and/or admission as to whether or not the present application contains any new matter in addition to the matter of its parent application(s).

### SUMMARY

In one aspect, a game system includes, but is not limited to: at least one game component configured for use in a game system by an individual player; at least one principal game unit including a port configured for communication with the at least one game component; at least one sensor, wherein the at least one sensor is configured to detect one or more analyte obtained from the individual player; and at least one signal output unit associated with the game component and configured to output signal in response to at least one sensed analyte. In one aspect, a game system includes, but is not limited to: at least one game component configured for use in a game system by an individual player; at least one sensor system operably connected to the at least one game component and configured to detect one or more analyte, the at least one sensor system including a signal transmitter; at least one signal detector configured to detect a signal transmitted from the at least one sensor system; and at least one principal game unit operably connected to the at least one signal detector, the at least one principal game unit including at least one signal transmitter configured to transmit a signal responsive to the at least one signal detector. In addition to the foregoing, other system aspects are described in the claims, drawings, and text forming a part of the present disclosure.

In one aspect, a method of determining the presence or absence of one or more analyte in at least one bodily fluid from an individual game player through a game interaction includes, but is not limited to, assessing at least one bodily fluid from an individual game player for one or more analyte with at least one sensor integral to at least one component of a game system, and indicating information from the assessment to at least one system user. In addition to the foregoing, other method aspects are described in the claims, drawings, and text forming a part of the present disclosure.

In one or more various aspects, related systems include but are not limited to circuitry and/or programming for effecting the herein-referenced method aspects; the circuitry and/or programming can be virtually any combination of hardware, software, and/or firmware configured to effect the herein-referenced method aspects depending upon the design choices of the system designer.

In addition to the foregoing, various other method and/or system and/or program product aspects are set forth and described in the teachings such as text (e.g., claims and/or detailed description) and/or drawings of the present disclosure.

The foregoing is a summary and thus may contain simplifications, generalizations, inclusions, and/or omissions of detail; consequently, those skilled in the art will appreciate that the summary is illustrative only and is NOT intended to be in any way limiting. Other aspects, features, and advantages of the devices and/or processes and/or other subject matter described herein will become apparent in the teachings set forth herein.

### BRIEF DESCRIPTION OF THE FIGURES

FIG. 1 illustrates aspects of a game system.

FIG. 2 depicts aspects of a game system.

FIG. 3A shows aspects of some embodiments of a game component.

FIG. 3B depicts aspects of some embodiments of a game component.

FIG. 4A illustrates aspects of some embodiments of a game component.

FIG. 4B depicts aspects of some embodiments of a game component.

FIG. 4C shows aspects of some embodiments of a game component.

FIG. 5 illustrates aspects of some embodiments of a game component.

FIG. 6 shows aspects of a game system.

FIG. 7 depicts aspects of a method.

FIG. 8 shows aspects of a method.

FIG. 9 illustrates aspects of a method.

FIG. 10 depicts aspects of the method illustrated in FIG. 9.

FIG. 11 shows aspects of the method illustrated in FIG. 9.

FIG. 12 depicts aspects of the method illustrated in FIG. 9.

FIG. 13 shows aspects of the method illustrated in FIG. 9.

FIG. 14 illustrates aspects of a game system.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

In some embodiments, a game system includes: at least one game component configured for use in a game system by an individual player; at least one principal game unit including a port configured for communication with the at least one game component; at least one sensor, wherein the at least one sensor is configured to detect one or more analyte obtained from the individual player; and at least one signal output unit associated with the game component and configured to output signal in response to at least one sensed analyte. In some embodiments, a game system includes: at least one game component configured for use in a game system by an individual player; at least one sensor system operably connected to the at least one game component and configured to detect one or more analyte, the at least one sensor system including a signal transmitter; at least one signal detector configured to detect a signal transmitted from the at least one sensor system; and at least one principal game unit operably connected to the at least one signal detector, the at least one principal game unit including at least one signal transmitter configured to transmit a signal responsive to the at least one signal detector.

The game systems and methods described herein can be used for the detection of analytes from individuals for whom conventional biological screening may be particularly unwanted or difficult, such as the elderly, infirm, children, or individuals suffering from psychiatric disorders. The game systems and methods described herein can be used for repeat monitoring of analytes from individuals, such as daily, weekly or monthly monitoring in a non-invasive and amusing or diverting format. For example, the game systems and methods described herein may be used to monitor analytes related to drug use, pathogenic infection, health or disease. For example, the game systems and methods described herein may be of used to monitor drug compliance in patients for whom clinical compliance is uncertain, such as psychiatric patients. See Cramer and Rosenbeck, “Compliance with medication regimens for mental and physical disorders,” Psychiatric Services, 49: 196-201 (1998), which is herein incorporated by reference. The game systems and methods described herein may be used in domestic, institutional and clinical settings. In some embodiments, additional physiological parameters are also evaluated from an individual game player.

The game systems and methods described herein can be implemented through electronic devices and units, mechanical devices and units, chemical devices and units, or a combination thereof. In some embodiments, all units of the systems described herein include electronic components configured to functionally operate together. In some embodiments, the units of the systems described herein may include electronic and mechanical components configured to functionally operate together. In some embodiments, the units of the systems described herein may include electronic and chemical components configured to functionally operate together. In some embodiments, the units of the systems described herein may include electronic, chemical and mechanical components configured to functionally operate together.

The game systems described herein are configured to detect one or more analytes associated with an individual game player. An “analyte,” as used herein, may include, but is not limited to, at least one biological marker, antibody, polypeptide, protein, complex, nucleic acid, cell, pathogen, lipid, alcohol, sterol, carbohydrate, metal, electrolyte, organic compound, nonorganic compound, organophosphate, drug, therapeutic, gas, taggant or pollutant. An analyte may include a metabolite, such as a metabolite of a substance provided with the game system, or a medicinal agent, or a metabolite indicative of a metabolic state. A game system may include a provided substance and the metabolite may include a metabolite of the provided substance. An analyte may include one or more metabolic compounds produced in whole or part by the individual game player's body. An analyte may include at least one moiety that is an indicator of a physiological state of an individual game player. An analyte may be assessed for its presence in bodily fluid from an individual game player. For example, a game system may be configured for detection of one or more analytes in salivary fluid from an individual game player. For example, a game system may be configured for detection of one or more analytes from the skin surface, such as from perspiration, of an individual game player. A game system may also, in addition to being configured to detect one or more analytes associated with an individual game player, be configured to detect one or more physiological parameters associated with the game player, such as pulse rate, blood oxygen levels, bodily activity or blood pressure. A game system may include one or more physiological sensors, such as a sensor configured to detect the body temperature, pulse rate, blood oxygen levels, bodily activity or blood pressure of an individual player.

A game component for use with a game system may include an individual marker for an individual game player, a game piece, a device operable during game play, and associated units. A game component is configured for use by a single individual player during game play activity, although each individual game player may use multiple game components during game play activity. For example, each individual game player may use a plurality of game component tokens or markers during game play activity, but the tokens or markers are not shared between players during game activity. For example, each individual game player may have his or her “own” handheld game component device for use during game play activity. For example, each individual game player may manipulate a specific and unique keyboard or game interface unit during game play activity. Different types of game activities may include different game components. A single game may include a single type of game component in addition to a principal game unit for game play activity, or it may include different types of game components. For example, game play activity for a specific game may include a single game component for each individual player, such as a handheld board marker or an individual game interface device. For example, game play activity for a specific game may include multiple game components for each individual player, such as markers, cards, interface devices, etc. For example, game play activity for a specific game may include one or more game interaction unit for each individual player, such as joysticks, keyboards, keypads, thumb pads, displays and touchscreens. Each individual game interaction unit may be operably attached, such as through wires. Each individual game interaction unit may be functionally attached, such as through being separate faces or units of a larger device.

A game system, including individual game components and a principal game unit, may be configured to facilitate the assessment of the presence or absence of one or more analytes from a particular bodily region of an individual game player. For example, a game system may be configured with game components shaped like candy, such as lollypops, candy sticks, or candy pieces, to encourage an individual player to suck on the game component and therefore to obtain analytes in salivary fluid. For example, a game system may be configured with game components shaped like a confection, such as ice pops or ice cream cones, to encourage an individual player to suck on the game component. Such activity may further be incorporated into the game itself, such as a game that rewards some actions by allowing an individual player to suck on a flavored game component as part of the game activity. For example, a game system may be configured with game components configured to encircle or enclose part of the body, such as the hand or arm, and therefore obtain perspiration fluid. A game system may include motion sensors incorporated in a feedback system with game play (see, for example, U.S. Patent Application No. 2008/0102953 to Schultz, titled “Toothbrush affecting game play,” which is herein incorporated by reference). A game system may include disposable modular elements, such as covers or wrappings. Disposable modular elements may be configured to protect, prior or subsequent to use, the portions of the game system that are configured to facilitate the detection of analytes associated with an individual game player. One or more modules of a game system, including game components and portions of game components, may be configured to be single-use and disposable.

Game systems and methods described herein may be configured to obtain samples of one or more analytes in bodily fluid, for example salivary fluid and perspiration fluid, of individual game players. As used herein, “salivary fluid” includes fluids routinely found in the oral cavity of an individual user, for example blood, breath condensate, breath aspirate, oral gas, crevicular fluid, transudate, exudate, gingival crevicular fluid, mucosal transudate or exudate, ingested remnants and mucus. Salivary fluid may include breath condensate or aspirate. For example, salivary fluids may include fluids originating with the mucus membranes of the esophagus, lungs or bronchial passages. For example, salivary fluids may include fluids containing dissolved or encapsulated gasses originating with the mucus membranes of the esophagus, lungs or bronchial passages. Game systems and methods described herein may be configured to obtain samples of analytes in the perspiration fluid of individual game players. As used herein, “perspiration fluid” includes fluids originating on the skin, such as through the sweat glands, and associated components such as salts, proteins, amino acids, peptides, nucleic acids and lipids. “Perspiration fluid” may also include cellular debris, hair follicles, and hairs, as well as skin-associated mites, bacteria, and pathogens. In some contexts, “perspiration fluid” may be referred to as “sweat.” In some situations, bodily fluids may be assessed even though they have dried, such as on the hands of an individual game player. For example, salivary fluid may be transferred to an individual game player's hands during coughing or sneezing, and the dried remnants subsequently assessed for the presence of mucus, bacteria, viruses, or other analytes.

Salivary fluid can provide a noninvasive source for biomarkers of systemic and local diseases and disorders. In addition to providing a copious supply of salivary fluids, the mouth can act as an access point to the gut, respiratory, and circulatory systems. In some individuals, such as children and the infirm, salivary fluids may be preferable to samples taken invasively. See, for example, European Patent Application Publication No. EP 1 397 997 A1 to Groschl and Rauh titled “Detection device,” and U.S. Pat. No. 6,022,326 to Tatum et al., titled “Device and method for automatic collection of whole saliva,” which are herein incorporated by reference. Studies illustrate the numbers and varieties of analytes that are available for testing in salivary fluids. See, for example: Kaufman and Lamster, “The Diagnostic Applications of Saliva-A Review”, Crit Rev Oral Biol Med, 13(2):197-212 (2002); Lawrence, “Salivary markers of systemic disease: noninvasive diagnosis of disease and monitoring of general health,” J. Can. Dent. Assoc. 68(3): 170-174 (2002); Li et al., “The oral fluid MEMS/NEMS chip (OFMNC): diagnostic and translational applications,” Adv. Dent. Res., 18: 3-5 (2005); “Salivary diagnostics, the ‘magic mirror’ to your health . . . at your personal computer,” ScienceDaily, Apr. 5, 2008; and Wong, “Salivary Diagnostics Powered by Nanotechnologies, Proteomics and Genomics,”J Am Dent Assoc., 137:313-321 (2006) which are herein incorporated by reference. In addition to compounds associated with the mouth and mucous membranes, such as immunoglobulin A (IgA), other analytes can be present at detectable levels, including markers of disease, drugs and alcohol. For example, salivary fluid has been used as a medium for the detection of HIV antibodies. See Hodinka et al., “Minireview: Detection of Human Immunodeficiency Virus antibodies in oral fluids,” Clin. & Diagn. Lab Immun., 5(4): 419-426 (1998), and Nishanian et al., “Oral fluids as an alternative to serum for measurement of markers of immune activation,” Clin. & Diagn. Lab Immun., 5(4): 507-512 (1998), which are herein incorporated by reference. Markers related to systemic health have also been measured in salivary fluids as an alternative source to serum. Hormones, antibodies, electrolytes, and cholesterol are just a few of the analytes that can be monitored in salivary fluids. See, for example: Hofman, “Human saliva as a diagnostic specimen,” J. Nutr., 131: 1621S-1625S (2001); Wong, “Oral Fluid NanoSensor Test (OFNASET)” grant 5U01DE017790-03 grant abstract; Karjalainen et al., “Salivary cholesterol of healthy adults in relation to serum cholesterol concentration and oral health,” J. Dent. Res. 76: 1637-1643 (1997); and Queyras and Carosi, “Non-invasive techniques for analyzing hormonal indicators of stress,” Ann 1st Super Sanita, 40(2): 211-221 (2004), which are incorporated herein by reference. Studies have also shown that markers of environmental chemical exposure are detectable in salivary fluids. See for example, Bauer “Saliva spits out information on chemical exposure,” Innovations Report, Oct. 24, 2003, which is herein incorporated by reference. Some analytes in the salivary fluids arise from gingival crevicular fluids, transudates or exudates. In addition, the oral mucosa is highly vascularised, which has led to the use of transmucosal access to the circulatory system.

The oral cavity is also directly connected to the digestive tract. This has allowed testing for stomach disorders, including the presence of Helicobacter pylori (H. pylori), a causal agent in stomach ulcers. The DNA of H. pylori can be isolated from salivary fluids taken from infected individuals. Additionally, under the right conditions, urea released by the pathogen can be measured in oral gases (see, for example, Pathak et al., “Urea breath test for Helicobacter pylori detection: present status,” Trop Gastroenterol. October-December; 25(4):156-61 (2004), which is herein incorporated by reference). Oral gases and condensates also provide a means of sampling lung exhalations to investigate pulmonary or systemic diseases. See, for example, U.S. Pat. No. 6,467,333 to Lewis et al., titled “Trace level detection of analytes using artificial olfactometry,” which is herein incorporated by reference. Nitric oxide, carbon monoxide, other volatile gases, as well as lipids, leukotrienes and eicosanoids are a few detectable markers of pulmonary diseases in oral samples (see, for example, “Exhaled Markers of Pulmonary Disease,” Kharitonov and Barnes, Am J Respir Crit Care Med, 163: 1693-1722, (2001), which is herein incorporated by reference). Respiratory diseases continue to be a major cause of morbidity and mortality throughout the world. In 2003, asthma alone affected 20.7 million American adults, or 9.7% of the total adult population (see, for example, Tables 3 and 4 as well as Appendix III, table V in the Summary Health Statistics for U.S. adults: National Health Interview Survey, 2003, published by the CDC) and 9.1 million children (12%; see, for example, Table 1 in the Summary Health Statistics for U.S. Children: National Health Interview Survey, 2003.) Game systems have been described as training mechanisms for lung function (see U.S. Patent Application No. 2008/0294061 to Wang and Li, titled “Health care gaming device and methods of using the same,” which is herein incorporated by reference).

Many analytes found in bodily fluids, such as perspiration fluid, salivary fluid and breath aspirate, have been shown to correlate with the presence of the analytes, or metabolic related species thereof, in serum. For example, many analytes have been shown to be detectable in perspiration fluid and/or salivary fluid as well as blood serum, and the relative concentrations in perspiration fluid and/or salivary fluid and serum and/or urine have been shown to correlate. Therefore, it is possible to convert values relating to the concentration of certain analytes in serum and/or urine to relative values for the same analytes in perspiration fluid and/or salivary fluid. For example, it has been shown that Δ⁹-tetrahydrocannabinol (THC) excretion in sweat correlates with the levels of THC in urine and serum from the same individuals (see Huestis et al., “Excretion of Δ⁹-Tetrahydrocannabinol in sweat,” Forensic Sci Int 174: 173-177 (2008), which is herein incorporated by reference). For example, individuals' immunization status for measles, mumps, and rubella have been shown to be detectable through antibodies in both serum and salivary fluids, with strong correlation between antibody levels in serum and salivary fluids (see Thieme et al., “Determination of measles, mumps, and rubella immunization status using oral fluid samples,” JAMA 272: 219-221 (1994), which is herein incorporated by reference). Therefore, information relating to the detection of analytes from salivary fluid or perspiration fluid as described herein may be converted to values corresponding to concentrations in blood serium and/or urine for many analytes. Such information may then be saved as part of an individual health history or record and used to compare relative levels of analytes present over time.

The detection of many analytes in salivary fluid have also been shown to be directly clinically relevant. For some analytes, concentration levels detected in salivary fluid have been shown to be at least as clinically relevant as concentration levels detected in blood serum or urine. For example, it has been shown that levels of phenyloin detected in salivary fluid are more accurate than serum levels for therapeutic dosing in the presence of valproate (see Knott et al., “Phenyloin-valproate interaction: importance of saliva monitoring in epilepsy,” British Medical Journal, 284: 13-16 (1982), which is herein incorporated by reference). For example, antibodies present in salivary fluid have been shown to be detectable after immunization (see Moldovenanu et al., “Human immune responses to influenza virus vaccines administered by systemic or mucosal routes,” Vaccine 13 (11): 1006-1012 (1995), which is herein incorporated by reference). For example, levels of nitric oxide (NO) as well as 8-isoprostane, INF-γ (INF-gamma), TNF-α (TNF-alpha), and IL-4 present in breath condensate have been shown to correlate with the presence and severity of asthma (see Robroeks et al., “Exhaled nitric oxide and biomarkers in exhaled breath condensate indicate the presence, severity and control of childhood asthma,” Clin. Exp. Allergy 37: 1303-1311 (2007) and Smith et al., “Use of exhaled nitric oxide measurements to guide treatment in chronic asthma,” N. Engl. J. Med. 352: 2163-73 (2005) which are incorporated herein by reference). For example, a bone resorption abnormality may be detected based on analytes present in perspiration fluid (see Kung et al., “Perspiration assay for bone resorption,” U.S. Pat. No. 5,661,039, which is herein incorporated by reference). Therefore information relating to the presence and relative concentrations of analytes may be saved directly into an individual's health or medical record for access by a system user, such as medical personnel or caregivers, for use in clinical evaluation.

Game systems and methods as described herein include one or more sensors configured to detect one or more analyte. For example, systems and methods as described herein may specifically identify an analyte found in salivary fluid, such as compounds associated with the mouth and mucous membranes, such as immunoglobulin A (IgA). An analyte may include an indicator of a physiological state, such as a disease state. In addition, other analytes can be present in salivary fluid and on the skin in perspiration fluid at detectable levels, including markers of disease, drugs and alcohol. For example, salivary fluid has been used as a medium for the detection of HIV antibodies. See Hodinka et al., “Minireview: Detection of Human Immunodeficiency Virus antibodies in oral fluids,” Clin. & Diagn. Lab Immun., 5(4): 419-426 (1998), and Nishanian et al., “Oral fluids as an alternative to serum for measurement of markers of immune activation,” Clin. & Diagn. Lab Immun., 5(4): 507-512 (1998), which are herein incorporated by reference. For example, it is possible to correlate the concentration of alcohol in exhaled breath and breath condensate with blood alcohol concentration. See, for example, the sheet titled “Scientific Method and Technology” under the header “Premium Digital Alcohol Breath Analyzer—Technology” regarding the AlcoHawk CA2000 and the AlcoHawk ABI Premium from Quick Medical, which is incorporated herein by reference. Markers related to systemic health have also been measured in salivary fluids as an alternative source to serum. Hormones, antibodies, electrolytes, and cholesterol are just a few of the analytes that can be monitored in salivary fluids. See, for example: Hofman, “Human saliva as a diagnostic specimen,” J. Nutr., 131: 1621S-1625S (2001); Wong, “Oral Fluid NanoSensor Test (OFNASET)” grant 5U01DE017790-03 grant abstract; Karjalainen et al., “Salivary cholesterol of healthy adults in relation to serum cholesterol concentration and oral health,” J. Dent. Res. 76: 1637-1643 (1997); and Queuras and Carosi, “Non-invasive techniques for analyzing hormonal indicators of stress,” Ann 1st Super Sanita, 40(2): 211-221 (2004), which are incorporated herein by reference. Studies have also shown that analytes that are markers of environmental chemical exposure are detectable in salivary fluids. See for example, Bauer “Saliva spits out information on chemical exposure,” PNNL news release, 2003, which is herein incorporated by reference. Some analytes in salivary fluids arise from gingival crevicular fluids, transudates or exudates.

Perspiration fluid can also provide a noninvasive source for biomarkers of systemic and local diseases and disorders. For example, analytes in perspiration fluid indicate recent drug use by an individual. See Barnes et al., “Excretion of methamphetamine and amphetamine in human sweat following controlled oral methamphetamine administration,” Clinical Chemistry 54: 172-180 (2008), and Kintz et al., “Sweat testing for heroin and metabolites in a heroin maintenance program,” Clinical Chemistry 43: 736-739 (1997), which are herein incorporated by reference. Analytes relating to drug use have been shown to persist for several days after drug use in some individuals (see, e.g. Huestis et al., ibid., which is herein incorporated by reference). Analytes in perspiration fluid may also indicate ingestion of other compounds, such as caffeine in relation to caffeinated beverages. See Kovacs et al., “Effect of caffeinated drinks on substrate metabolism, caffeine excretion, and performance,” J Appl Physiol 85: 709-715 (1998), which is herein incorporated by reference. Analytes in perspiration fluid may indicate ingestion of alcoholic beverages (see Philips, “Sweat-patch test for alcohol consumption: rapid assay with an electrochemical detector,” Alcoholism: Clinical and Experimental Research, 6(4): 532-534 (1982), which is herein incorporated by reference). In addition, physiological problems may be detectable through analytes in perspiration fluid. For example, a bone resorption abnormality may be detected based on analytes present in perspiration fluid (see Kung et al., “Perspiration assay for bone resorption,” U.S. Pat. No. 5,661,039, which is herein incorporated by reference). The total volume of perspiration fluid may also be used as an indicator of neurophysiology. For example, perspiration has been shown to correlate with a clinical diagnosis of erythromelalgia (see Davis et al., “Thermoregulatory sweat testing in patients with erythromelalgia,” Arch Dermatol. 142: 1583-1588 (2006), which is herein incorporated by reference.)

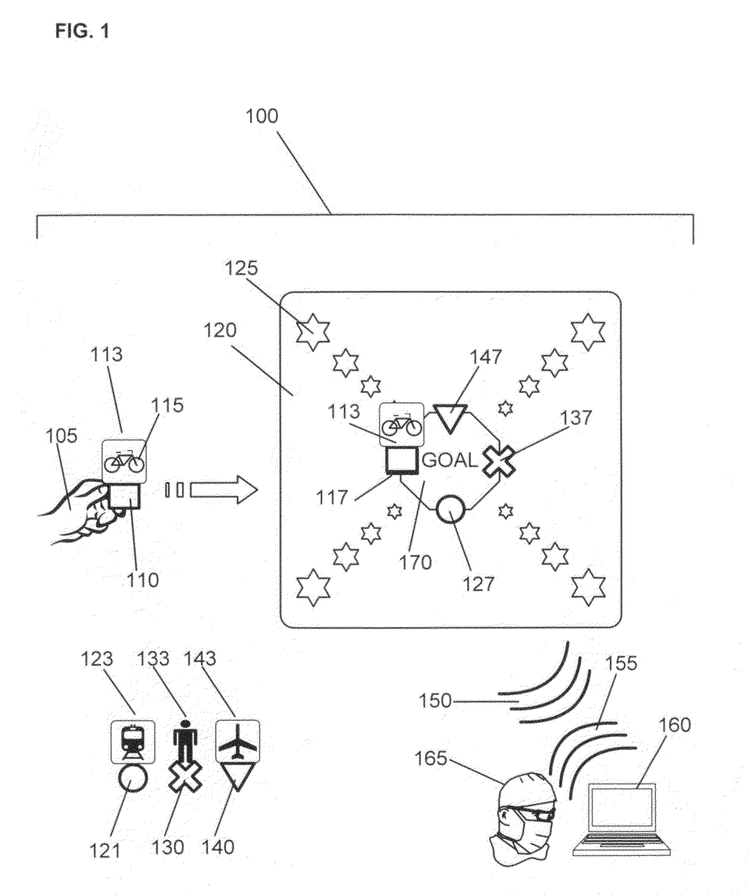

With reference now to FIG. 1, shown is an example of a game system that may serve as a context for introducing one or more processes and/or devices described herein. The use of the same symbols in different drawings typically indicates similar or identical items. FIG. 1 depicts a game system 100 including a plurality of game components 113, 123, 133, 143 configured for use in a game system 100 by an individual game player 105. In some embodiments there may be a single individual player and in some embodiments there may be multiple players. Some game systems may be configured to accept multiple individual players in some play functions and single individual players in other play functions. An individual player may utilize a single, distinct game component during game play activity. An individual player may utilize more than one game component during game play activity. A game system may include electronic components, non-electronic components, or a combination of both electronic components and non-electronic components. A game component 113, 123, 133, 143 may include an identifier, logo, symbol or similar depiction 115 to distinguish a unique game component or set of game components from other game components. For example, the game components 113, 123, 133, 143 illustrated in FIG. 1 include depictions of a bicycle, a train, a human figure and an airplane, respectively. A game component 113, 123, 133, 143 may include at least one unique identifier, such as visible identifier like a depiction 115, a unique shape or coloring, or a bar code. A game component 113, 123, 133, 143 may include at least one unique identifier, such as a transmission module with a radio frequency identification (RFID) tag or another unique transmitted signal that serves to identify a specific game component 113, 123, 133, 143. A game component 113, 123, 133, 143 may include at least one transmitter. A game component 113, 123, 133, 143 may be configured to at least partially encircle part of the individual player's body. For example, a game component 113, 123, 133, 143 may be configured as a ring, bracelet, arm band, necklace, headband, anklet or a component configured to encircle the ear of an individual player. A game component 113, 123, 133, 143 may include a region configured to encircle an individual player's body part completely or partially, such as a hand, finger, or arm. For example, a game component may contain a region configured as a complete or partial covering for the hand or finger, such as a complete or partial glove, ring or knuckle cover. For example, a game component may include a region configured to partially or completely encircle the wrist of an individual game player, such as an arm sweatband, bracelet, or wristband. A game component may be integrated into a device worn on a band circling the wrist, such as a watch. For example, a game component may include a region configured to partially or completely encircle the head of an individual game player, such as a hat, head sweatband, headband, visor or head cloth. A game component 113, 123, 133, 143 including a region configured to encircle an individual player's body part may operate to hold the game component in place during game play, to orient the individual player's body relative to the game component for increasing the assessment of bodily fluid, or for ease of detection of a physiological parameter of the individual user (i.e. blood pressure or pulse rate). A game system 100 may include a pulse oximeter or other monitoring device, such as in a game component 113, 123, 133, 143 configured to encircle part of the individual player's body, such as a finger or wrist. See Patel, “Nintendo Wii Vitality Sensor detects your pulse,” posted Jun. 2, 2009, which is herein incorporated by reference. A game component 113, 123, 133, 143 may include electrodes configured to sense changes in skin potential for evaluation of sympathetic skin response (SSR, or galvanic skin response: see Illigens and Gibbons, “Sweat testing to evaluate autonomic function,” Clin, Auton. Res. 19:79-87 (2008), which is herein incorporated by reference). A game component 113, 123, 133, 143 may include multiple sub-components and may be flexible, configured to be manipulated, or mobile. For example, a game component 113, 123, 133, 143 may include buttons, dials, sticks, or similar functional components. For example, a game component 113, 123, 133, 143 may include a joystick, a handheld electronic game unit, or a display. A game component may be operably attached to a principal game unit, such as through a wire or wireless connection. A game component may be distinct from the at least one principal game unit. A game component may be discrete from the at least one principal game unit. A game component may be integral to a principal game unit, for example a region, keyboard, or face of a principal game unit that is configured for use by an individual player. A game component 113, 123, 133, 143 may include a region configured to accept the breath of an individual player, such as a mouthpiece attached to a tube or straw and configured to sequester breath condensate from an individual player. A game component 113, 123, 133, 143 may include modular elements, such as disposable covers or components. A game component 113, 123, 133, 143 and/or a game system 100 may include electronic circuitry. A game component 113, 123, 133, 143 may be devoid of electronic circuitry. A game system may include additional components, such as dice, cards, spinners or buzzers configured to enhance game play activity.

A game system 100 is configured for use in one or more games. A “game,” as used herein, includes an activity that is designed to be an amusement or a pastime. Each game may be played by a single individual at a time, or by two or more individuals in concert or parallel. Where a game is played by two or more individuals, the game play activity may include the players interacting, such as by taking turns, working collaboratively towards a common goal, or competing. Where a game is played by two or more individuals, the game play activity may include the players acting independently of each other. For example, a game system 100 may interact with more than one individual game player 105 independently of any other game player using the game system 100 at the same time as the individual game player. A game system 100 includes one or more game components 113, 123, 133, 143 and at least one principal game unit 120 that are used by at least one individual game player 105 during game play activity. Multiple types of games may be played with different game systems such as those described herein. For example, some embodiments of the game systems described herein may be utilized in playing active games, such as those that include running, jumping, dancing, balancing, navigating an obstacle course, or other similar activities. For example, a game component 113, 123, 133, 143 may be integrated into a sweatband configured to be worn around the head, neck or wrist of an individual game player 105. The related game activity may include running a certain distance followed by placement of the game component 113, 123, 133, 143 in alignment with a principal game unit 120. For example, some embodiments of the game systems described herein may be utilized in playing games tied to public sporting events. For example, some embodiments of the game systems described herein may be utilized in playing games tied to professional sports. For example, an individual game player 105 may hold a game component 113, 123, 133, 143 during a sporting event, and be encouraged to suck on the game component when an athlete, sports team or team member scores or achieves some other target in the sporting event. At the conclusion of the sporting event or at a time point in the event (such as, for example, halftime, a penalty, an intermission, after an inning, the end of a match, etc.) an individual game player 105 may be encouraged to place the game component 113, 123, 133, 143 in association with a principal game unit 120. For example, an individual game player 105 may be required to suck on a game component 113, 123, 133, 143 to initiate an electronic game with a game system 100, and to then encouraged to place the game component 113, 123, 133, 143 in association with a principal game unit 120 to continue game play (such as to obtain extra points or additional time, to advance the game play, to increase the level of game play, to access additional game features, etc.). In some embodiments, medicinal agents may be incorporated with game play. For example, an individual game player 105 may be encouraged to drink a medicinal agent during and as part of game play, to place a game component 113, 123, 133, 143 into their oral cavity, and at some later time place the game component 113, 123, 133, 143 in association with a principal game unit 120 as part of the game play activity. Thus, the effectiveness or metabolization of a medicinal agent may be evaluated by the game system. Thus, the administration of a medicinal agent to an individual player/patient may be evaluated by the game system.

A game system, including a game component 113, 123, 133, 143 and a principal game unit 120, may be manufactured in part or entirety from a substantially rigid material, for example a hard plastic or fibrous composite. A game system, including a game component 113, 123, 133, 143 and a principal game unit 120, may include at least one pliable material. A game component 113, 123, 133, 143 may include, for example, at least one natural gum base, artificial gum base, acacia, carageenan, plastic, elastomeric polymer, polyisobutylene, or paraffin. For example, a game component 113, 123, 133, 143 may include a pliable material that is configured to bend or reform due to physical pressure within an individual player's oral cavity, such as from sucking or chewing activity. A game component 113, 123, 133, 143 may include at least one salivary fluid collection unit, which may be configured to passively collect salivary fluid, such as being configured to bend from the physical pressure of an individual player sucking or chewing on the game component. A game system, including a game component 113, 123, 133, 143 and a principal game unit 120, may include at least one material that is encapsulated, such as a material configured for timed release or durability during storage. A game system, including a game component 113, 123, 133, 143 and a principal game unit 120, may include a least one portion that is dehydrated prior to contact with bodily fluid. A game system, including a game component 113, 123, 133, 143 and a principal game unit 120, may include sterile packaging.

A game system, including at least one game component 113, 123, 133, 143 and a principal game unit 120, may include one or more modules. The modules may be configured for removal, replacement, recharge, cleaning and/or refurbishment. In some embodiments, modules may be configured for a single use. In some embodiments, modules may be configured to be disposable. In some embodiments, modules may be fabricated from recyclable or biodegradable materials to facilitate disposal. For example, a game component 113 may have a modular removable cover configured for removal and replacement. A modular removable cover of a game component 113 may be fabricated from a plastic material that is generally accepted by recyclers. Many recyclers currently accept various types of plastics, in particular polyethylene terephthalate (PET: often labeled as recycling number 1) or high-density polyethlene (HDPE: often labeled as recycling number 2). For example, a principal game unit 120 may include a modular removable cover, which may be fabricated from a plastic material that is generally accepted by recyclers. For example, a modular removable cover, such as of a game component 113 and/or of a principal game unit 120, may be fabricated from a biodegradable plastic. Commonly available types of biodegradable plastics include hydro-biodegradable plastics (HBP) and oxo-biodegradable plastics (OBP). A biodegradable plastic may be configured for disposal in an actively managed compost environment, for example a commercial compost setting. For example, a game component 113, 123, 133, 143 may include at least one module, such as an interior module including matrix components, a power source, or microcircuitry, which is configured for removal and replacement. For example, a game component 113, 123, 133, 143 may include at least one reservoir configured to accrue, store and/or dispense a substance. For example, a game component 113, 123, 133, 143 may include one or more salivary fluid collection units configured to accrue and store salivary fluid. For example, a game component 113, 123, 133, 143 may include at least one reservoir configured to hold at least one medicinal agent. For example, a game component 113, 123, 133, 143 may include at least one reservoir configured to store at least one medicinal agent. For example, a game component 113, 123, 133, 143 may include at least one reservoir configured to hold at least one medicinal agent prior to active or passive release of the medicinal agent. For example, a game component 113, 123, 133, 143 may include at least one reservoir configured to hold at least one flavorant. For example, a game component 113, 123, 133, 143 may include at least one reservoir configured to store at least one flavorant. For example, a game component 113, 123, 133, 143 may include at least one reservoir configured to hold at least one flavorant prior to active or passive release of the flavorant. For example, a game component 113, 123, 133, 143 may include one or more taggant reservoirs configured to responsively or passively release a taggant. For example, a principal game unit 120 may include at least one module configured to be removed and cleaned, such as one or more regions 117, 127, 137, 147 configured for placement of one or more game components 113, 123, 133, 143, which may include a removable cover or interior. For example, a principal game unit 120 may include at least one module, such as a detection unit or transmitter, which is configured for removal and replacement. Additionally or alternately, at least a portion of the game system may be configured for a single use.

Additionally, any part or all components of the system 100 may be provided in a sterile form and/or the system may include sterile packaging for at least a portion of the system, including a game component 113, 123, 133, 143 and a principal game unit 120. For example, there may be one or more modules that may be swapped out, removed, or replaced and the newly incorporated modules may include sterile packaging prior to incorporation and/or after removal. For example, there may be one or more modules that may be swapped out, removed, or replaced and the removed modules may be placed in sterile packaging prior to further analysis, examination, or disposal.

Portions of the system 100 described herein may be configured to be cleaned or have microbial contamination removed, such as before disposal or reuse. For example, the system may be made up of modules fabricated from materials that are structurally resistant to degradation by cleaning or sterilization products or methods. A game component 113, 123, 133, 143, modules or portions thereof may be configured to be sterilizable through conventional techniques such as UVC exposure, autoclaving, chemical or steam disinfection. Similarly, one or more portions of an external device may be configured to be sterilizable. For example, it may be desirable to sterilize a region 117, 127, 137, 147 of a principal game unit 120 configured for direct contact with a game component 113, 123, 133, 143. In some embodiments, a game component 113, 123, 133, 143 and/or a principal game unit 120 may include UVC capability to self-sterilize. For example, one or more UVC-emitting light source may be incorporated into a game component 113, 123, 133, 143 and/or a principal game unit 120, and configured to sterilize the relevant surfaces before use or between uses. Similarly, one or more steam-emitting instruments may be incorporated into a game component 113, 123, 133, 143 and/or a principal game unit 120, and configured to clean or eliminate pathogens on the relevant surfaces before use, after use, or between uses. One or more chemical disinfectants may also be incorporated into a game component 113, 123, 133, 143 and/or a principal game unit 120, and configured to clean or eliminate pathogens on the relevant surfaces before use, after use, or between uses. For example, one or more chemical disinfectants may be incorporated into a reservoir configured to controllably release or passively release the chemical disinfectants. For example, one or more chemical disinfectants may be incorporated into the structure of the game component 113, 123, 133, 143 and/or a principal game unit 120, or a cover or module thereof. For example, silver, calcium phosphate, triclosan, or silane-based antimicrobial agents may be incorporated into a module or unit of the game system 100.

In some embodiments, a game component 113, 123, 133, 143 may be manufactured in whole or in part from one or more gel or gel-like material such as a hydrogel, a hydrosol, a sol-gel, xerogel, an aerogel, a smart gel, a hydrocarbon gel, a ferrogel, a colloid, a superporous gel, a responsive gel, or other gel made from natural polymers, synthetic polymers, or a combination or composite thereof. A game component 113, 123, 133, 143 may include a chewable, pliant substance, such as one containing, for example, a natural or synthetic gum base, such as those used in chewing gums, like acacia or carrageenan; or a paraffin wax, soft plastic, or an elastomeric polymer like polyisobutylene. A game component 113, 123, 133, 143 may include at least one pliable material. A game component 113, 123, 133, 143 may be any shape and size, as required by the game activity and the functions of the game component 113, 123, 133, 143.

In some embodiments, a game component 113, 123, 133, 143 or a module or portion of the game component may be configured to allow for swallowing of the game component by an individual. Although swallowing of a game component is not envisioned as part of routine game activity, accidental or unintentional ingestion may occur in some circumstances and a game component 113, 123, 133, 143 or module or portion thereof may be configured to minimize hazard in the case of ingestion. A game component 113, 123, 133, 143, module or portion thereof configured to allow for swallowing may, for example, be configured in a size and shape to minimize choking hazard or intestinal blockage in the case of ingestion. A game component 113, 123, 133, 143, module or portion thereof configured to allow for swallowing may, for example, be manufactured from materials that are non-toxic, non-irritating, and stable when passed through an individual's gastrointestinal tract. Similarly, a game component 113, 123, 133, 143, module or portion thereof may be configured to minimize the possibility of accidental ingestion of the entirety or a part of a game component 113, 123, 133, 143, such as by fabricating a game component 113, 123, 133, 143, module or portion thereof in a size and shape unlikely to be swallowed by a game player or other individual. A game component 113, 123, 133, 143, or module may be configured to be structurally sound, with minimal regions that are likely to break off and potentially be ingested.

A game component 113, 123, 133, 143 may include at least one flavorant. A flavorant may be included in a coating or covering over the outside of the game component. A flavorant may be included in a reservoir within the game component configured for passive or active release. For example, the game component 113, 123, 133, 143 may include at least one flavorant or flavoring agent such as those common to the food industry. For example a game component 113, 123, 133, 143 may include at least one flavorant incorporating flavoring agents and a carbohydrate, gelatin or oil based compound. A flavorant may be a natural flavorant, such as the essential oil, oleoresin, essence or extractive, protein hydrolysate, distillate, or any product of roasting, heating or enzymolysis, which contains the flavoring constituents derived from a spice, fruit or fruit juice, vegetable or vegetable juice, edible yeast, herb, bark, bud, root, leaf or any other edible portions of a plant, meat, seafood, poultry, eggs, dairy products, or fermentation products thereof, whose primary function in food is flavoring rather than nutritional. A flavorant may be an artificially created flavorant. A flavorant may be a combination of compounds, including natural and artificially created compounds. A flavorant may include salts, sugars, artificial sweeteners, or flavor enhancers. For example, a game component 113, 123, 133, 143 may be covered entirely or partially with a flavorant. For example, the at least one flavorant may include a carbohydrate, gelatin or oil based coating on the surface of the game component 113, 123, 133, 143. For example, the at least one flavorant may be coated, dried or glazed onto the exterior of the game component 113, 123, 133, 143 or a cover 300. A flavorant may be included in an oleaginous material that also includes granulated gasified candy, such as described in U.S. Pat. No. 4,275,083 to Colten et al., titled “Gasified candy enrobed with oleaginous material,” which is herein incorporated by reference. In some embodiments, the flavorant may be initially located in an indentation, reservoir or internal region of the game component 113, 123, 133, 143 that is configured to release the flavorant through mechanical force, such as an individual sucking, chewing, or rubbing on the game component 113, 123, 133, 143. In some embodiments, the flavorant may be initially located in an indentation, reservoir or internal region of the game component 113, 123, 133, 143 that is configured to release the flavorant through the addition of heat, such as an individual holding the game component 113, 123, 133, 143 or placing the game component 113, 123, 133, 143 adjacent to the skin of an individual player and thereby transferring body heat from the individual player to the game component 113, 123, 133, 143. In some embodiments, the flavorant may be initially located in an indentation, reservoir or internal region of the game component 113, 123, 133, 143 that is configured to release the flavorant through the addition of bodily fluid, such as with a covering configured to dissolve in the presence of salivary fluid or perspiration fluid. A flavorant may be desiccated prior to contact with bodily fluid. In some embodiments, the flavorant may be initially located in an indentation, reservoir or internal region of the game component 113, 123, 133, 143 that is configured to release the flavorant in response to a condition, such as a temperature or pH. In some embodiments, the flavorant may be initially located in an indentation, reservoir or internal region of the game component 113, 123, 133, 143 that is configured to release the flavorant in response to a sensor. For example, a sensor may be configured to initiate an electrical signal that results in the release of flavorant. For example, a sensor may include a swellable gel that changes the conformation of a reservoir and thereby releases flavorant.

In some embodiments, the at least one flavorant is of a type expected to influence an individual, for example being configured to emit a pleasing flavor for a length of time, configured to lose a pleasing flavor after a particular length of time or use, or configured to emit an unpleasant flavor after a particular length of time or use. In some embodiments, the flavorant may be targeted to one or more group of users, for example a flavorant with sour flavor may be desirable to encourage a sufficient quantity of salivary fluid in the oral cavity in individuals with characteristically dry mouths. Citric acid, for example, has been shown to stimulate salivary fluid expression or production. See U.S. Pat. No. 6,102,872 to Doneen et al., titled “Glucose detector and method,” which is herein incorporated by reference. For example, a flavorant that tastes like candy, such as a bubble gum or cotton candy flavor, may be desirable for use with children. For example, a flavorant tasting like mint or spice flavor may be desirable for use with adults. In some embodiments, the at least one flavorant is configured to change chemical composition during contact with the skin of an individual player relative to at least one of time, duration of physical pressure, presence of a target material, or presence of an amount of a target material. For example, a flavorant may be configured with a limited quantity of flavoring agent and therefore configured to lose flavor after a finite length of time. For example, a flavorant may be configured with a limited quantity of a dissolvable flavoring agent, such as a sugar-based compound configured to dissolve after contact with a particular amount of perspiration in combination with the physical pressure of an individual player holding the game component 113, 123, 133, 143. For example, a flavorant may be encapsulated with a carbohydrate substrate which is configured to dissolve after contact with sufficient perspiration. See, for example, U.S. Pat. No. 6,746,529 to Witteveen et al., titled “Stable, spray-dried composition in a carbohydrate substrate and process for obtaining said composition,” which is herein incorporated by reference. A flavorant may be incorporated into an emulsion.

The game system also includes at least one principal game unit 120, including a port configured for communication with the at least one game component 113, 123, 133, 143. A principal game unit 120 may include markings 125, such as depictions and symbols relevant to a specific game to be played. For example, a principal game unit 120 configured for playing checkers may include markings 125 such as a black and red checkerboard. For example, a principal game unit 120 configured for playing solitaire may include markings 125 depicting playing cards in a row. A principal game unit 120 may include a region 170 designed for players to place their individual game components 113, 123, 133, 143 at the end of the game or a portion of the game, such as a goal, finish, end or score region 170 of the principal game unit 120. A principal game unit 120 may include modular elements, such as disposable covers or components. A principal game unit 120 may include one or more regions 117, 127, 137, 147 configured for placement of one or more game components 113, 123, 133, 143. A principal game unit 120 may include one or more regions 117, 127, 137, 147 configured to pair with a game component 113, 123, 133, 143. For example, one or more regions 117, 127, 137, 147 configured for placement of one or more game components 113, 123, 133, 143 may include markings configured to match the size and shape of one or more game components 113, 123, 133, 143. For example, one or more regions 117, 127, 137, 147 configured for placement of one or more game components 113, 123, 133, 143 may include markings configured to pair with one or more game components 113, 123, 133, 143, such as indentations or raised elements of the principal game unit 120 configured to mate with indentations or raised elements of one or more game components 113, 123, 133, 143. For example, FIG. 1 depicts game component 113 including a base 110 configured as a rectangle, and principal game unit 120 including a region 117 configured to pair with the base 110 rectangle of game component 113. Similarly, FIG. 1 illustrates game components 123, 133, 143 including base 121, 130, 140 elements shaped as a circle, X-mark, and triangle, respectively. The principal game unit 120 depicted in FIG. 1 illustrates regions 127, 137, 147 configured for placement of one or more game components 123, 133, 143 including base 121, 130, 140 elements shaped as a circle, X-mark, and triangle. In some embodiments, there may be one or more regions of a principal game unit 120 configured to pair with all or some of the game components of a system. For example, there may be one or more regions of a principal game unit 120 configured to mate with or conform to all or some of the game components of a system. Some embodiments may include individual regions of a principal game unit 120 configured to match individual game components such as depicted in FIG. 1 as regions 117, 127, 137 and 147 configured to mate with elements 110, 121, 130, 140 of individual game components 113, 123, 133, 143.

A game system 100 may include electronic circuitry, including electronic circuitry in the principal game unit 120. A game system 100 may be configured with circuitry to send signals 150 to a network, including elements such as a distant network computing device 160. A game system 100 may be configured to receive signals 155 from a network, including a distant network computing device 160. Although wireless signals 150, 155 are depicted in FIG. 1, in some embodiments a principal game unit 120 may transmit or receive signals through a wire connection or other physical conduit. One or more system user 165, such as a medical professional, caregiver, or public health official, may access information relating to the game system through the network, such as through a network computing device 160. A network computing device 160 may, for example, indicate to a system user 165 when results are available, options for display of information, or relevant medical information. A network computing device 160 may store information for later access by a system user 165. A unit of the game system 100 may include a user interface device such as a keyboard, touchscreen, display screen, touchpad, E-ink device, or auditory signal generator/receiver. For example, a network computing device 160 and/or a principal game unit 120 may include a user interface.

A game component 113, 123, 133, 143 may include at least one sensor configured to detect one or more analyte. At least one sensor may be integral to at least one game component 113, 123, 133, 143. A principal game unit 120 may include at least one sensor configured to detect one or more analyte. At least one sensor may be integral to at least one principal game unit 120. A “sensor” as used herein, includes a unit that specifically identifies a substance, such as an analyte, and generates a signal that the identification has been made. A sensor may include a gas or chemical sensor, or an optical, acoustic, or electric sensor. A sensor may be an electrochemical sensor. A sensor may be a biological sensor. In some embodiments, there may be only one sensor in a game system 100 or there may be a plurality of sensors in the game system.

In some embodiments, there may be a sensor system in a game system 100, wherein the sensor system includes at least one sensor. In some embodiments, there may be a sensor system in a game system 100, wherein the sensor system includes at least one sensor operably connected to a signal transmitter. A game component 113, 123, 133, 143 or a principal game unit 120 may be operably connected to at least one sensor system, the sensor system configured to detect one or more analyte and including a signal transmitter. A sensor system may include at least one sensor configured to detect at least one analyte in salivary fluid. For example, a sensor system may include at least one sensor configured to detect antibodies, pathogens, or drug analytes in salivary fluid. A sensor system may include at least one sensor configured to detect at least one analyte in perspiration fluid. For example, a sensor system may include at least one sensor configured to detect salts, caffeine, or drug analytes in perspiration fluid. A sensor system may include at least one sensor configured to detect at least one analyte in breath aspirate. For example, a sensor system may include at least one breathing tube configured to condensate fluid from breath aspirate prior to analysis by a sensor. A sensor system may include at least one recognition element such as those described herein. The at least one recognition element may be configured to recognize one or more chemical substance. A sensor system may be configured to be directly responsive to the one or more analyte. A sensor system may be configured to be indirectly responsive to the one or more analyte. For example, a sensor system may be configured to be responsive to a metabolite of the one or more analyte. For example, a sensor system may be configured to be responsive to a taggant, such as a taggant associated with the analyte.

Depending on the embodiment, a sensor system may include a plurality of sensors, which may be of a single type or multiple types. A sensor system may also include at least one power source, at least one antenna, and one or more display. A sensor system may be integral to another unit of a game system 100, such as a principal game unit or a game component. A sensor system may include a reservoir. A sensor system may include a gel or matrix containing sensor. A sensor system may be operably connected to another unit of a game system 100, such as a principal game unit or a game component. A sensor system may include a signal transmitter. A principal game unit may include at least one detector configured to detect a signal transmitted from the at least one sensor system.

Depending on the embodiment, various possible types of sensors may be utilized within a sensor system, a game component 113, 123, 133, 143 or integral to or operably connected to the principal game unit 120. A game system 100 may include at least one sensor system including at least one sensor. Multiple types of sensors are described herein as exemplary types. One or more sensor may include, for example, at least one optical sensor, acoustic sensor, electromagnetic sensor, magnetic sensor, electrophoretic sensor, electrochemical sensor, biochemical sensor, microfluidic sensor, magnetic resonance sensor, piezoelectric sensor, surface plasmon resonance sensor, optical microsensor array, surface enhanced raman spectrometer (SERS), laser, ion flow tube, metal oxide sensor (MOS), infrared spectrophotometer, acoustic wave sensor, colorimetric tube, conductive-polymer gas sensor, chemoresistor, selective resonance sensor, gas chromatograph, mass spectrophotometer, or magnetic resonance sensor. A sensor may include at least one gel. As an example of a visible light, UV or IR sensor, see the MiScope® Handheld Digital Microscope, available from Forensics Source (Jacksonville Fla.). A sensor may be optical and rely on frustrated total internal detection (FTIR) of magnetic particles, see Gelfand, “Device Offers a Roadside Dope Test,” MIT Technology Review Online Edition Aug. 4, 2009, which is herein incorporated by reference. A sensor may be fabricated in micrometer or nanometer scale, such as described by Beck et al., “Nanoelectrochemical transducers for (bio-) chemical sensor applications fabricated by nanoimprint lithography,” Microelectronic Engineering, 73-74(1): 837-842 (2004), which is herein incorporated by reference.

A sensor is configured to generate a signal in response to detection of an analyte. The signal generated by a sensor may be, for example, an electrical, visual, magnetic, acoustic, vibrational, heat, light (including infrared (IR) or ultraviolet (UV)), radio frequency (RF) or electromagnetic (EM) radiation signal. In some embodiments, the sensor may be configured to generate a signal directly in response to an analyte. For example, a sensor may be configured to include a luminescent compound that is released in the presence of an analyte, in which case the signal may be the presence of the luminescent compound or light emitted from the luminescent compound. In some embodiments, a sensor may include a matrix that includes a gel configured to be responsive to a substance, wherein the gel is configured to emit a signal when the substance is detected. A signal may include a chromatic, fluorescent, luminescent, or aromatic signal. A signal may include energy-emitting particles or radioactive particles. A signal may include a releasable taggant. Examples of signal systems include a polymerized crystalline colloidal array responsive to glucose. See, for example, U.S. Pat. Nos. 6,187,599 and 6,544,800 to Asher et al., titled “Polymerized crystalline collidal arrays,” and U.S. Pat. No. 7,105,352 to Asher et al., titled “Intelligent polymerized crystalline colloidal array carbohydrate sensors,” which are herein incorporated by reference. The Asher group at the University of Pittsburgh has also described the fabrication of polymerized crystalline colloidal arrays. See the attached printout of the Asher Laboratory materials titled “Colloid Group,” printed on Jul. 31, 2009, which are incorporated herein by reference. Chemical sensors utilizing physical transducers with integrated piezoresistive signals have been described, such as by Potyrailo et al., “Chemical sensors based on micromachined transducers with integrated piezoresistive readout,” Analytical Chemistry, 78 (16): 5633-5638 (2006), which is herein incorporated by reference.

A sensor may be operably attached to a transducer of the signal from the sensor. Among transducers, examples include acoustic transducers, composite piezoelectric transducers, conformal transducers, flexible transducers, flexible ultrasonic multi-element transducer arrays, flexible ultrasound transducers, immersible ultrasonic transducers, integrated ultrasonic transducers, microfabricated ultrasound transducers, piezoelectric materials (e.g., lead-zirconate-titanate, bismuth titanate, lithium niobate, piezoelectric ceramic films or laminates, sol-gel sprayed piezoelectric ceramic composite films or laminates, piezoelectric crystals, and the like), piezoelectric ring transducers, piezoelectric transducers, ultrasonic sensors, ultrasonic transducers, and the like. A transducer can include a single design where a single component outputs one single waveform at a time, or may be compound where two or more components are utilized in a single transducer or in multiple transducers thereby allowing multiple waveforms to be output sequentially or concurrently. For a review of transducers including cantilever designs, see Lavrik et al., “Cantilever transducers as a platform for chemical and biological sensors,” Review of Scientific Instruments, 75(7): 2229-2253 (2004), which is herein incorporated by reference.

A sensor may include at least one electronic chip sensor, which may be configured as a modular unit. An electronic chip sensor may be configured for reuse, multiple use, or single use. An electronic chip sensor may be removable, swappable, or replacable, such as an electronic chip sensor configured as a module. Other instrumentation associated with the game component 113, 123, 133, 143 may interact with the electronic chip sensor, such as one or more microfluidic device, which may be integrated into a principal game unit 120. Examples of electronic chips that may be configured for use with some embodiments includes immunoassay microchips and electrochemical DNA sensor chips. See, for example, Dill et al., “Immunoassays and sequence-specific DNA detection on a microchip using enzyme amplified electrochemical detection,”J. Biochem. Biophys. Methods 5: 181-187 (2004) and Drummond et al., “Electrochemical DNA sensors,” Nature Biotech. 21: 1192-1199 (2003), which are incorporated herein by reference. An electronic chip sensor may be configured for use with electronic, acoustic, or wireless technology to communicate remotely with a principal game unit 120 through a port, such as when a port is configured to be a receiver. See, for example: Yazawa et al., “A wireless biosensing chip for DNA detection,” ISCC 2005 30.6; Baker, “Beaming Biodata,” MIT Technology Review May 2005 (online edition); Heim, “Lab on a swab,” MIT Technology Review, Aug. 29, 2005; and Hitachi Ltd. News Release “Development of the world's first RFID sensor chip for DNA analysis—SNPs in DNA detected using chip and reader only,” Feb. 10, 2005, which are incorporated herein by reference. In some embodiments, a game component 113, 123, 133, 143 or a principal game unit 120 including one or more electronic chip sensor may include a power source, which may be configured to supply power to an electronic chip sensor through an electric transmission element, such as wires. A power source for a portion of the system 100, such as for a game component 113, 123, 133, 143 or a principal game unit 120 may include one or more rechargeable elements. A power source for a portion of the system 100, such as for a game component 113, 123, 133, 143 or a principal game unit 120 may include one or more transmitted power sources. See U.S. Patent Application No. 2005/0143787 to Boveja titled “Method and system for providing electrical pulses for neuromodulation of vagus nerve(s), using rechargeable implanted pulse generator,” which is herein incorporated by reference.

In embodiments in which a game component 113, 123, 133, 143 includes a sensor, the game component or a principal game unit 120 may include a detector configured to recognize a signal from a game component 113, 123, 133, 143. A detector may be configured to detect a signal directly generated by the sensor, or a signal generated by a signal output unit. For example, a principal game unit 120 may include a detector configured to recognize a signal from a game component 113, 123, 133, 143 through a port, for instance a glass window, a receiver, or a gas port. A port may be included in a region 117, 127, 137, 147 of the principal game unit 120 configured to pair with a game component. A port may be included in a region 170 of the principal game unit 120 indicated, for example, as a ‘goal,’ ‘finish’ or scoring region of the principal game unit 120. A detector configured to recognize a signal from a game component 113, 123, 133, 143 may be integrated into the principal game unit 120 without a specific port. A detector configured to recognize a signal from a game component 113, 123, 133, 143 may be integral to the game component. For example, a detector may be configured to detect signals directed generally to the principal game unit, such as sound waves or electromagnetic (EM) signals. As an example of a hand-held tandem mass spectrophotometer that may be integrated into a device, such as a principal game unit, see Gao et al., “Design and characterization of a multisource hand-held tandem mass spectrophotometer,” Anal. Chem. 80: 7198-7205 (2008), which is herein incorporated by reference. As an example of a gamma-beta radiation detector, see the Ion Ferret™ device available from Overhoff Technology Corporation (Milford Ohio), the 2009 brochure for which is herein incorporated by reference. As an example of a liquid scintillation counter detector, see the Innovative Technology Summary Report titled “Lumi-scint Liquid Scintillation Counter,” OST/TMS ID 2311, July 2001, which is herein incorporated by reference. As an example of a carbon dioxide or carbon monoxide detector and thermometer, see the AQ2000 hand-held analyzer available from KIMO (France).

An electronic chip sensor integral to a game component 113, 123, 133, 143 may be configured to interface directly with a principal game unit 120 through a port configured for communication, such as a port configured for communication through electronic circuitry. An electronic chip sensor integral to a game component 113, 123, 133, 143 may be configured to interface directly with a principal game unit 120 through a port configured for communication, such as a port configured for communication through one or more wavelengths of light. An electronic chip sensor integral to a game component 113, 123, 133, 143 may be configured to interface directly with a principal game unit 120 through a port configured for communication, such as a port configured for communication through vibration. In some embodiments, the interface between an electronic chip sensor and a principal game unit 120 may be enhanced through a surface component of the game component 113, 123, 133, 143. For example, a surface component of the game component 113, 123, 133, 143 may be configured to amplify or transmit one or more wavelengths of light. For example, a surface component of the game component 113, 123, 133, 143 may be configured to amplify or transmit vibration.

In some embodiments, a sensor is configured to be included within a game component 113, 123, 133, 143. A sensor may be configured to detect at least one analyte in salivary fluid. A sensor may be configured to detect at least one analyte in perspiration fluid. A sensor may be configured to detect at least one analyte in breath aspirate. Depending on the embodiment, many possible types and configurations of the one or more sensor integral to a game component 113, 123, 133, 143 may be utilized, including one or more array. Depending on the embodiment, a sensor may be utilized that is very small, such as a sensor or array that is configured to fit within a game component 113, 123, 133, 143. In some embodiments, the sensor is a chemical sensor. See, for example, Snow et al., “Chemical Detection with a Single-Walled Carbon Nanotube Capacitor,” Science 307:1942-1945 (2005), which is incorporated herein by reference. A sensor incorporated within a game component 113, 123, 133, 143 should be of a size and shape able to be configured for complete enclosure within the game component 113, 123, 133, 143. Furthermore, a sensor incorporated within a game component 113, 123, 133, 143 should be operable at ambient temperatures and conditions during game play. Some types and configurations of sensors, therefore, are not suitable for inclusion within a game component 113, 123, 133, 143. In embodiments wherein a sensor is incorporated within a game component 113, 123, 133, 143, a principal game unit 120 may be configured to detect at least one signal from the sensor. For example, a port in the principal game unit may include one or more devices configured to detect a signal transmitted from the sensor system.

A game component 113, 123, 133, 143 may include at least one detector configured to detect a signal from the at least one sensor. For example, a game component 113, 123, 133, 143 may include a sensor including a swellable, analyte-responsive gel and a detector including a pressure sensor. For example, a game component 113, 123, 133, 143 may include an aptamer-based electrochemical sensor and also an integrated detector including a voltammeter. For example, a game component 113, 123, 133, 143 may include a sensor including a chemiluminescent module and a detector configured to respond to the emitted light. Circuitry configured to propagate and/or transmit a signal may also be included within a game component 113, 123, 133, 143.

Some embodiments include at least one signal output unit associated with the game component 113, 123, 133, 143 and configured to output signal in response to a sensed analyte. For example, a signal output unit may include a device that amplifies a signal from the sensor. For example, a signal output unit may include a transducer. For example, if a sensor within a gel binds an analyte and results in the change in shape or size of the gel, a signal output unit including a piezoelectric component may be configured to output signal in response to the change in shape or size of the gel. For example a signal output unit may include a device that converts a signal from the sensor into a signal of a form configured to be detected by a principal game unit. For example, a sensor integral to a game component 113, 123, 133, 143 may bind an analyte and result in a change in color of a component of the sensor. A signal output unit associated with the game component 113, 123, 133, 143 may include an optical reader that detects the color change and, in response, transmit a signal of sound, visible light, UV or IR to the principal game unit. A game component 113, 123, 133, 143 may include at least one detector configured to detect a signal from the at leats one sensor, wherein the at least one detector is operably connected to the at least one signal output unit.

Some embodiments include a sensor system. A sensor system may include, inter alia, one or more sensors, detectors, signal output units, or transceivers as described herein. For example, in an embodiment wherein a sensor system is integral to an individual game component, the sensor system may include a sensor and a signal output unit. For example, in an embodiment where a sensor system is integral to a principal game unit, a sensor system may include a sensor, a detector and a transceiver. Other combinations would be relevant to different embodiments, as fitting the requirements of any specific embodiment, such as sensitivity, cost, fabrication, size, durability, disposal parameters, and compatibility with a larger medical system, such as an electronic hospital records system.

In some embodiments, at least one sensor is configured to be integral to a principal game unit 120. Depending on the embodiment, many possible types and configurations of the one or more sensor integral to a principal game unit 120 may be utilized. In some embodiments, a principal game unit 120 may be configured to be portable, such as a handheld or table-top device. In some embodiments, a principal game unit 120 may be included in a larger fixture or device, such as a medical testing apparatus or machine. In some embodiments, a principal game unit 120 may be coextensive with a device with other functionalities, such as a cell phone, computing device, media presentation device, digital recording device (e.g. MP3 player) or personal digital assistant (PDA). A sensor integral to a principal game unit 120 may, therefore, be of various sizes, weights and configurations depending on the embodiment. A principal game unit 120 may include a display, such as a light or display screen, configured to be responsive to at least one sensor. For example, a display may be configured to indicate that a signal has been received from a sensor, or that a sensor has recognized a specific analyte. A principal game unit 120 may include for example, at least one natural gum base, artificial gum base, acacia, carageenan, plastic, elastomeric polymer, polyisobutylene, or paraffin. A principal game unit 120 may include at least one reservoir. The at least one reservoir may include a gel configured for slow release of a chemical compound, such as a scented compound, a reagent required for operation of a sensor or detector, or a taggant.

A principal game unit 120 may include at least one signal detector configured to detect a signal transmitted from at least one sensor system operably connected to the at least one game component 113, 123, 133, 143. For example, at least one signal detector may be integral to the principal game unit. For example, a principal game unit 120 may be operably connected to at least one signal detector, and the at least one principal game unit may include at least one signal transmitter configured to transmit a signal responsive to the at least one signal detector. For example, FIG. 1 illustrates signals 150 from at least one signal transmitter configured to transmit a signal responsive to the at least one signal detector, wherein the signals 150 are configured to be received by a network computing device 160. A principal game unit 120 may include at least one signal transmitter. A principal game unit 120 may include at least one unique identifier. For example, a principal game unit 120 may include a unique signal transmitter that identifies that specific principal game unit in a larger system that may include many components, including many principal game units. For example, a principal game unit 120 may include a radio frequency identification (RFID) tag. For example, a principal game unit 120 may be colored, marked, or shaped in a unique manner for the game system. For example, a principal game unit 120 may include a display indicating a unique identifier for that principal game unit 120 (e.g. “Joe's game unit”).

A game system 100 may include at least one signaling element configured to signal contact of a game component 113, 123, 133, 143 with an individual game player 105 relative to at least one of time, presence of a target substance, or presence of an amount of a target substance. A signaling element may function to emit a signal after contact between a game component 113, 123, 133, 143 and an individual game player 105 has occurred, for example to signal a system user 165 that the system is operating. Depending on the embodiment, a system including a signaling element may be configured to signal contact with an individual game player 105 at a specific point. Depending on the embodiment, a system including a signaling element may be configured to signal lack of contact with an individual game player 105, or insufficient contact, at a specific point. For example, a system including a signaling element may be configured to signal contact with an individual game player 105 relative to heat, such as body heat of an individual game player 105. For example, a game system 100 including a signaling element may be configured to signal contact with an individual game player 105 relative to at least one of time, presence of a target material, or presence of amount of a target material. A target material may include, for example, the analyte detected by the sensor, or a reference or additional substance. For example, the target material may be water, and the signaling element may be configured to indicate that a fluid containing water has been detected. As salivary fluid and perspiration fluid contain some portion of water, a system including such a signaling element may function to indicate to a system user 165 that the system has sufficient contact with an individual game player 105 for relevant function. For example, a game system 100 may include a signaling element and a timekeeping device operably attached to a fluid sensor and a signal emitter, configured so that a system user 165 would be alerted that fluid had or had not been detected during a preset period of time. For example, a game system 100 may include at least one signal emitter. A signal emitter may include, for example, a light, sound, or vibration emitting device. For example, a game system 100 may include a signaling element operably attached to a sensor so that the sensor will cause a signal to be generated when an analyte is present or absent after a particular period of time. For example, a game system 100 may include a signaling element operably attached to a sensor so that the sensor will cause a signal to be generated when the sensor has detected a quantity of the analyte after a particular period of time. For example, the signaling element may be configured to emit a signal when more than a threshold level of an analyte, such as cocaine, has been detected in salivary fluid. Signaling elements may include, for example, electronic elements such as an acoustic wave generator, a vibration emitter or an electric light. For example, a signal emitter may beep, vibrate or flash light after the game system 100 has been in contact with an individual game player for a preset period of time and therefore indicate to a system user 165 that the game system 100 is operational. Signaling elements may include, for example, electronic elements such as a small electric shock emitter, or a transmitter to send data to an external device, such as a computing device 160. The computing device 160 may be configured to include a signaling function, for example by displaying text on a display such as “system operational” or by illuminating a specific indicator in a user interface. Signaling elements may include chemical elements such as chemical dyes, inks, chromogens, or fluorogens. For example, a game component 113, 123, 133, 143 may be configured with a color indicator that changes color in the presence of a target material. See, for example, U.S. Patent Application No. 2002/0044891 to Miller et al., titled “Food Quality Indicator Device,” which is herein incorporated by reference. Signaling elements may include flavorants, such as a signaling element configured to emit a flavorant with an unpleasant flavor after a threshold quantity of an analyte has been detected. Signaling elements may include a volatile chemical compound, such as a signaling element configured to emit an odorant with an unpleasant scent after a threshold quantity of an analyte has been detected.

A game system may include a transceiver system. For example, a game system may include a transceiver system including a transmitter and a receiver in distinct components of the game system. For example, a principal game unit may include a transmitter and a remote computing device may include a receiver. For example, a game component may include a transmitter and a principal game unit may include a receiver. For example, a game component may include a receiver and a principal game unit may include a transmitter. A game component may include a transceiver system configured to receive signals from the at least one principal game unit. A principal game unit may include at least one transceiver system configured to transmit signals from the at least one principal game unit to the at least one game component. A principal game unit may include at least one transceiver system configured to receive signals from at least one game component. A principal game unit may include at least one transceiver system configured to receive signals from at least one external network system. A principal game unit may include at least one transceiver system configured to transmit signals from the at least one principal game unit to at least one external device, such as a computing device, network device, external antenna, or other game system unit. A transceiver system may transmit and receive signals of a number of types, including light, sound, electromagnetic (EM) radiation, vibration, and IR.

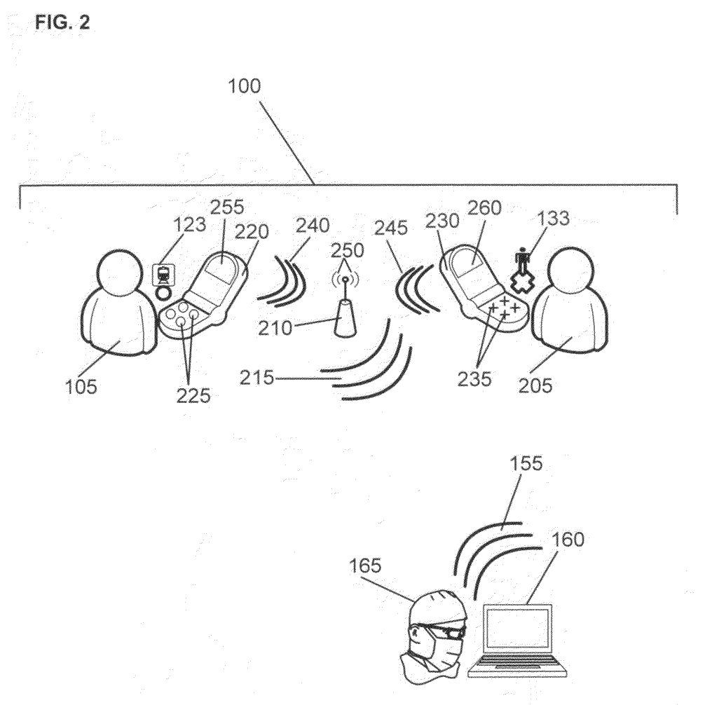

FIG. 2 illustrates further aspects of a game system. A game system 100 may include a plurality of principal game units 220, 230. The multiple principal game units 220, 230 may include unique identifiers configured to distinguish the principal game units. For example, each principal game unit 220, 230 may be a distinct color or include specific markings. For example, each principal game unit 220, 230 may include a bar code. For example, each principal game unit 220, 230 may include a RFID tag. Each principal game unit 220, 230 may be configured for use with one or more game components 123, 133. As depicted in FIG. 2, a principal game unit 220, 230 may be configured for use with one or more specific game components 123, 133. For example, FIG. 2 depicts principal game unit 220 configured with region 225 configured to pair with a portion of game component 123. Although these paired regions of the game component 123 and principal game unit 220 are depicted as physical shapes in FIG. 2, in some embodiments the paired regions may include visual markings or electronic system pairings. For example, a game component 123 may include a RFID tag and the corresponding principal game unit 220 may include a sensor configured to respond to that particular RFID tag, such as by being configured to only accept signals from a game component 123 containing that unique RFID tag. Similarly, FIG. 2 illustrates a second principal game unit 230 including regions 235 configured to pair with one or more specific game component 133. Each principal game unit 220, 230 may be utilized in game play by an individual game player 105, 205. In some embodiments, a game component 123, 133 and/or a principal game unit 220, 230 may be configured to respond to a specific individual game player 105, 205, such as a game player wearing a RFID identifier that indicates a specific individual game player 105, 205 or an individual game player 105, 205 entering a specific code into the game system. Each individual game player 105, 205 may use his or her own principal game unit during game play activity. One or more sensors in the game system 100 may similarly be configured to respond to a specific individual game player 105, 205, such as being configured to switch on or switch off in response to a signal that a specific individual game player 105, 205 is using the system.

A principal game unit 120 may include at least one sensor configured to detect one or more bodily fluid analyte. For example, a principal game unit 120 may include at least one sensor configured to detect one or more analyte obtained from salivary fluid or perspiration fluid. A principal game unit 120 may include at least one sensor configured to detect one or more analyte in bodily fluid. For example, a principal game unit 120 may include at least one sensor configured to detect one or more analyte contained in salivary fluid or perspiration fluid. A principal game unit 120 may include at least one detector configured to detect a signal from the at least one sensor. For example, a principal game unit 120 may include a microfluidic device with an arrayed immunosensor including mucin antibodies. See Tang et al., “Magnetic control of an electrochemical microfluidic device with an arrayed immunosensor for simultaneous multiple immunoassays,” Clinical Chemistry 53: 1323-1329 (2007), which is herein incorporated by reference. Such a microfluidic device may be operably attached to a reservoir collection unit, such as a conduit, configured to allow the egress of salivary or perspiration fluid from a reservoir within a game component 113, 123, 133, 143. A principal game unit 120 may include at least one detector configured to detect a signal from the at least one sensor, such as a light emitter of a specific wavelength and corresponding light detector.

FIG. 2 depicts a game system 100 including a plurality of principal game units 220, 230 and game components 123, 133, wherein the principal game units 220, 230 are configured to communicate with a network transmission element 210 by means of signals 240, 245, 250. FIG. 2 depicts principal game units 220, 230 transmitting signals 240, 245 to a network transmission element 210 which correspondingly transmits signals 250 to the principal game units 220, 230. The network transmission element 210 may also transmit and receive signals 215, 155 from other network elements, such as a network computing device 160. A network computing device 160 may indicate relevant information, such as results or data, to a system user 165. A network computing device 160 may indicate relevant information to a system user 165 through, for example, a display, indicator lights, a monitor, or auditory communications. Although the signals 155, 215, 240, 245 depicted in FIG. 2 are illustrated as wireless signals, in some embodiments there may be wires or other conduits configured to allow signals to be exchanged between the system devices. Although the network transmission element 210 and the network computing device 160 are depicted as separate devices in FIG. 2, in some embodiments they may be attached or incorporated into a single unit. A game system 100 may include at least one power source operably attached to the at least one principal game unit 220, 230. A game system 100 may include at least one antenna operably attached to the at least one principal game unit 220, 230. A game system 100 may include at least one user interface device operably attached to the at least one principal game unit 220, 230. For example, a game system 100 may include a joystick, keyboard, touchscreen, keypad, switches, dials or other user interface devices operably attached to the at least one principal game unit 220, 230.

A game system 100 may include one or more display unit. For example, a game system 100 may include at least one display operably attached to a network computing device 160. A game system 100 may include one or more display unit configured to be responsive to the at least one sensor. For example, a principal game unit 220, 230 may include a display 255, 260 configured to be responsive to the at least one sensor. For example, the display unit may be configured to show a color, pattern or message when an analyte is detected by the sensor. For example, a display unit may be configured to activate when a sensor is active, i.e. so that game play activity may be initiated or continued. For example, a display unit operably attached to a network computing device 160 may be configured to activate, display a color, pattern or message in response to a signal originating with the at least one sensor. A game system 100 may include one or more display unit configured to be responsive to the at least one principal game unit. For example, a principal game unit 220, 230 may include a display 255, 260 configured to be responsive to a module of that principal game unit 220, 230. For example, a principal game unit 220, 230 may include a display 255, 260 configured to be responsive to one or more regions 225, 235 configured to pair with one or more specific game component, such as a display 255, 260 configured to activate or display a color, pattern or message when a specific game component 123, 133 is placed in proximity to the one or more regions 225, 235. For example, a principal game unit 220, 230 may include a display 255, 260 configured to be responsive to signals received by the principal game unit 220, 230. For example, a principal game unit 220, 230 may include a display 255, 260 configured to be responsive to input from at least one individual game player 105, 205. A game system 100 may include one or display unit operably attached to a network computing device 160 configured to be responsive to the at least one principal game unit 220, 230. For example, a game system 100 may include one or more display unit operably attached to a network computing device 160 configured to activate, display a color, pattern or message in response to a signal received from the at least one principal game unit 220, 230.

A game system 100 may include at least one physiological sensor. For example, a game system 100 may include a game component 123, 133 configured to encircle the fingertip of an individual game player 105, 205 wherein the game component includes a pulse oximeter. For example, a game system 100 may include a game component 123, 133 configured to encircle the wrist, arm or fingertip of an individual game player wherein the game component includes a digital sphygmomanometer. Results from the physiological sensor may be stored in memory or transmitted to a computing device 160, such as a part of a network.

Although system users 165, 745, 845 are shown/described herein as a single illustrated figure, those skilled in the art will appreciate that system users 165, 745, 845 may be representative of a human user, a robotic user (e.g., computational entity), and/or substantially any combination thereof (e.g., a user may be assisted by one or more robotic agents) unless context dictates otherwise. Those skilled in the art will appreciate that, in general, the same may be said of “sender” and/or other entity-oriented terms as such terms are used herein unless context dictates otherwise.

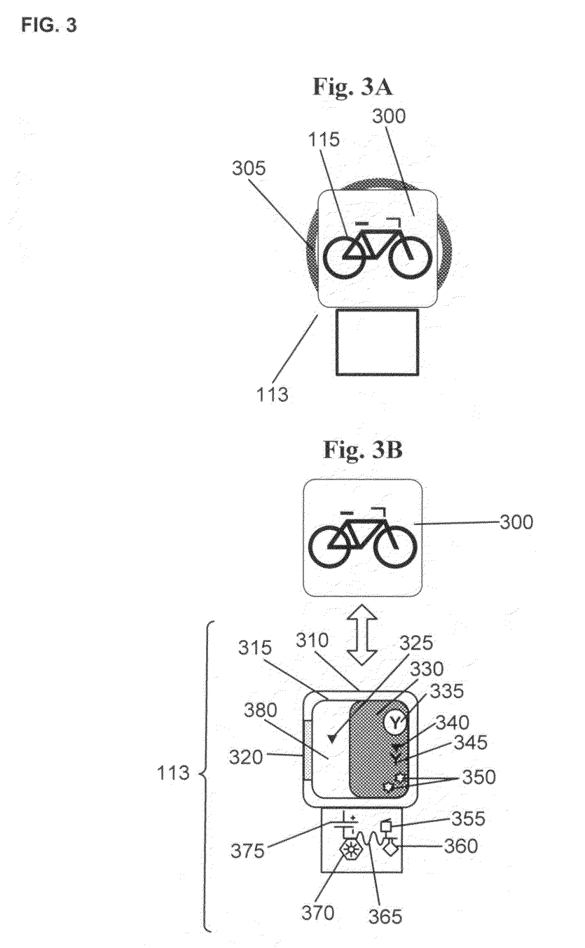

FIG. 3 depicts aspects of a game component 113. A game component 113 may include a removable game component cover 300, which may be configured to envelop all or a portion of the game component 113. As shown in FIG. 3A, a removable covering may include a symbol 115, marking or logo configured to distinguish a particular removable game component cover 300 or game component 113. A game component 113 may include a coating 305, such as an emulsion, covering all or part of the game component 113 or removable game component cover 300. A coating 305 may include one or more stabilizers or protective elements. See, for example, U.S. Pat. No. 7,022,514 to Vodyanoy et al., titled “Use of acacia gum to isolate and preserve biological material,” and U.S. Patent Application No. 2003/0138939 A1 to Vodyanoy et al., titled “Use of acacia gum to isolate and preserve biological material,” which are incorporated herein by reference. A coating 305 may include one or more flavorants.

One or more flavorant may be included in the game component 113 within an emulsion or encapsulation, such as a coating 305 and/or incorporated within the material forming the game component. A flavorant may be of a type designed to influence the user, for example a pleasant flavor that would encourage its use. The flavorant may be of a type responsive to a condition, such as time passed or the presence of a target material. Time-dependent flavorants are well-known in the food and candy industries. Multiple emulsions and encapsulations with differing properties as well as double emulsions are capable of enhanced responsiveness and may be included. See, for example, Utada et al., “Monodisperse Double Emulsions Generated from a Microcapillary Device,” Science 308:537-541 (2005), which is herein incorporated by reference. The one or more flavorant might be configured for responsiveness, such as to heat from a game player's mouth or hand. For instance, flavorant may be released through passive displacement or flavorant may be released through the operation of a more active element, such as release from a substance-responsive gel. A coating 305 may include gasified candy components configured to create a short-term sizzling or popping sensation in an individual player's mouth, as described in U.S. Pat. No. 4,275,083 to Colten et al., titled “Gasified candy enrobed with oleaginous material,” which is herein incorporated by reference.

A game component 113 may include at least one compound configured to be physiologically incorporated into the body of a user, and may be configured to retain the at least one compound until a predetermined condition. For example, a game component 113 may include at least one compound configured to be physiologically incorporated into the body of a game player within at least one matrix 330. For example, a game component 113 may include at least one compound configured to be physiologically incorporated into the body of a game player within a coating 305 such as an emulsion or encapsulation. For example, a matrix may be configured to retain a taggant or medicinal agent until the game component 113 is contacted with salivary fluid or perspiration. For example, a matrix 330 or a coating 305 may be configured to retain a provided substance, such as a medicinal agent, until the game component 113 has been in contact with salivary fluid for a preset period of time, such as the time required to dissolve an emulsion or encapsulation layer. See, for example, U.S. Pat. No. 6,746,529 to Witteveen et al., titled “Stable, spray-dried composition in a carbohydrate substrate and process for obtaining said composition,” which is herein incorporated by reference. A coating 305 may be configured to include a gaseous provided substance, including isotopically labeled carbon dioxide. For example, U.S. Pat. No. 4,275,083 to Colten et al., titled “Gasified candy enrobed with oleaginous material,” which is herein incorporated by reference, describes gas bubbles incorporated into edible candy coating.

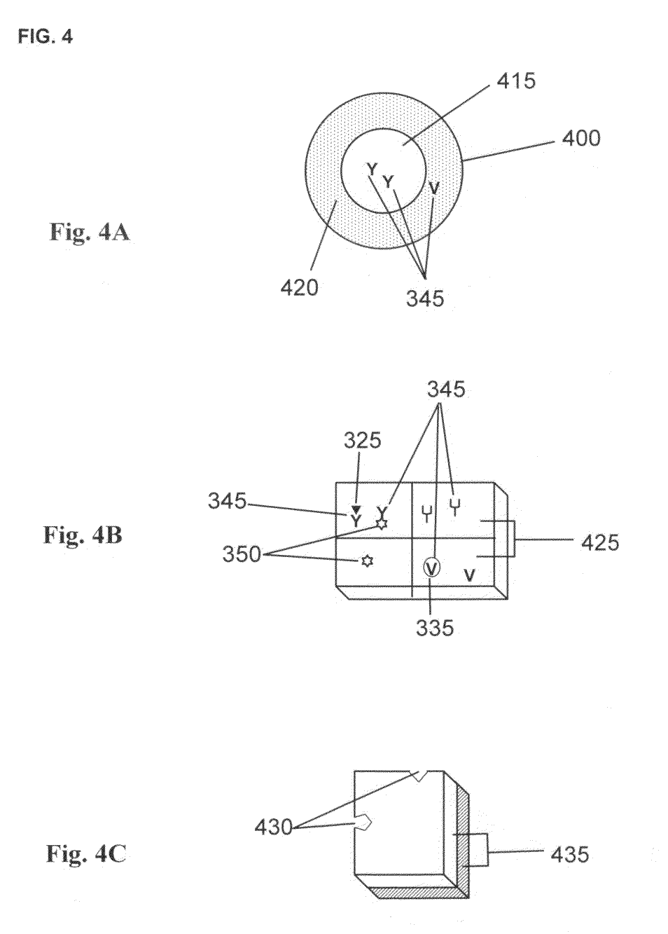

FIG. 3B illustrates additional aspects of a game component 113. As shown in the top portion of FIG. 3B, a removable game component cover 300 may be removed from the external surface of a game component 113. The interior of a game component 113 is depicted in cross-section in the lower portion of FIG. 3B. In some embodiments, a game component 113 includes an outer wall 310 and an inner wall 315. A game component 113 may be configured to allow access to the interior of the game component 113 through a permeable area of a game component cover 300 such as a gas permeable membrane such as a polysiloxane, and/or a selective medium 320. A game component may include at least one selective medium 320. The selective medium may include material configured as a screen with openings for the passage of some components, such as an analyte 325, and the exclusion of others, such as larger particulates or macromolecules. A selective medium may be configured as a layer such as illustrated 435 in FIG. 4C. A selective medium may be configured to filter out, for example, debris, cells, molecules of a range of sizes (including those above or below a specific range), charged molecules, or any other undesirable material, even excess moisture, while being configured to allow some other substances to pass through. Such a selective medium could be made from any of a number of materials including charcoal or cellulose; a synthetic polymer such as but not limited to polyethylene, polycarbonate, nylon, polyester, polysiloxane, or polypropylene; or a hydrogel, or a monolayer or bilayer of lipids, and a selective medium could include a protein. For example, a selective medium may include a layer made of cellulose configured with pores sized to allow diffusion of certain sized molecules, a hydrogel film of a type that swells at a certain pH, a gas-permeable membrane or a hydrophobic lipid bilayer. See, for example, “A hydrogel-based CO2 sensor,” Herber and Olthuis, MESA+ Institute for Nanotechnology, University of Twente, which is incorporated herein by reference. For example, a selective medium may include biocompatible membranes such as those described in U.S. Pat. No. 6,258,870 to Hubbell et al., titled “Gels for encapsulation of biological materials,” which is herein incorporated by reference. A selective medium could also or instead include one or more active transporter, such as a porin or ion transporter. A selective medium may be configured as a module or layer.

FIG. 3B illustrates an internal cross-section view of an exemplary game component 113. As illustrated in FIG. 3B, a game component 113 may include an internal region 380 including multiple features. A game component 113 may include a matrix 330. An internal region 380 may include one or more modular elements configured to passively collect salivary fluids and/or constituents thereof, including cells or other biologics, within a matrix 330 in an internal region 380. In some embodiments, the game component 113 may include a passive sampling container, and one or more gel or gel-like materials could include an absorbent made from one or more material like those mentioned herein, which may be dehydrated in its initial state prior to contact with salivary fluid or perspiration. In some embodiments, the game component 113 may include a passive sampling container that is empty prior to use, and may be fabricated with negative pressure prior to use to encourage flow of bodily fluid into the passive sampling container. A game component 113 may include at least one material from the list including absorbent, adsorbent, proteoglycan, charged polymer, polylysine, silica gel, alumina gel, and ion exchange resin. The matrix 330 may include an absorbent, like cotton, cellulose, natural or artificial sponge. The matrix 330 may include one or more gel, like a hydrogel, a hydrosol, a sol-gel, a xerogel, an aerogel, a hydrocarbon gel, a natural polymer gel, a synthetic polymer gel, a ferrogel, a colloid, a responsive gel, a superporous hydrogel or microparticle gel. A matrix 330 may be in a dehydrated form prior to contact with bodily fluid, such as salivary fluid or perspiration fluid. Many types of porous hydrogels may be utilized, such as those used in the wound dressing described in U.S. Pat. No. 6,372,248, to Qin et al., titled “Dehydrated Hydrogels,” which is incorporated herein by reference. An internal region 380 may include, possibly as a coating on the surface of the internal region 380, a synthetic or natural adsorbent material of a type that promotes the adhesion of one or more constituent in a salivary fluid or perspiration, like a cell or a protein. For example, a game component 113 may include a proteoglycan or a charged polymer such as polylysine. Other retaining materials could be included, such as semi-specific or non-specific adsorbents, such a silica (SiO₂) or alumina (Al₂O₃)— containing gel or an ion exchange resin, including as part of the matrix 330.

The matrix 330 may be fabricated from any number of materials or composites as appropriate to an embodiment, such as, but not limited to, a natural gel like agarose, a natural and/or synthetic polymer gel, hydrogel, or colloid, and may include a gum base such as an acacia gum. See, for example, U.S. Pat. No. 7,022,514 to Vodyanoy et al., titled “Use of acacia gum to isolate and preserve biological material,” and U.S. Patent Application No. 2003/0138939 A1 to Vodyanoy et al., titled “Use of acacia gum to isolate and preserve biological material,” which are incorporated herein by reference. A matrix 330 may, instead or in addition, be a lipid monolayer or bilayer, as in a micelle or liposome, and may be anchored to a internal region 380 through a nonorganic tether. See, for example, “Design of Supported Membranes Tethered via Metal-Affinity Ligand-Receptor Pairs,” Radler et al., Biophysical Journal 79:3144-3152 (2000), which is herein incorporated by reference. A matrix 330 may be configured as one or more film or layer. A matrix 330 may include at least one of a hydrogel, hydrosol, sol-gel, hydrocarbon gel, natural polymer gel, synthetic polymer gel, lipid, colloid, encapsulation or emulsion. A matrix 330 may be configured as a plurality of spheres, such as micro- or nano-spheres. Such spheres might include protein cages, liposomes, synthetic hybrid cerasomes, microspheres or nanospheres of one or more natural and/or synthetic polymer, including dendrimers. See, for example, Katagiri et al. “Creation of asymmetric bilayer membrane on monodispersed colloidal silica particles,” Colloids Surf B Biointerface, 38(3-4):149-53 (2004), which is incorporated herein by reference. For example, a matrix 330 may include at least one ligand affinity resin with or without a conjugated peptide or antibody such as those that are commonly used in chromatography and purification. For example a matrix 330 may include at least one ionophore as the recognition element presented on microspheres within the matrix. See, for example, U.S. Pat. No. 7,247,489 to Bakker, titled “Ion detecting microspheres and methods of use thereof,” which is incorporated herein by reference. For example, distinctly from a recognition element configured as a separate agent, a recognition element may be a recognition site molecularly imprinted within a matrix itself or a part thereof, such as a molecular mimetic. See, for example: U.S. Pat. No. 6,670,427 to Ulbricht et al., titled “Template-textured materials, methods for the production and use thereof;” Ye et al., “Molecularly imprinted polymers as antibody and receptor mimics for assays, sensors and drug discovery;” Anal Bioanal Chem. 378(8):1887-1897 (2004); and Peppas et al., “Polymers and gels as molecular recognition agents,” Pharm Res. 19(5):578-587 (2002), which are incorporated herein by reference.

One or more modular units of a game component 113 may be configured to store compounds within a matrix 330, which may be located within an internal region 380. For example, the matrix 330 may store a compound configured to be released. For example, the matrix 330 may be configured to store a recognition element 345. A matrix 330 may include at least one of a carbohydrate, alginate, protein, protein cage, lipid, phospholipid, liposome, cerasome, oil, emulsion, polymer, spheres, microspheres, or nanospheres. See U.S. Patent Applications Nos. 2004/0115132, 2006/0204444 and 2007/0059245 to Young et al., titled “Protein cages for the delivery of medical imaging and therapeutic agents,” and U.S. Patent Application No. 2006/0292174 to de los Rios and Oh titled “Self-assembling nanoparticle drug delivery system,” which are herein incorporated by reference. A game component 113 may include a hydrogel including hybrid materials, for example a hydrogel containing a hybrid protein-polysaccharide material. See U.S. Pat. No. 6,821,331 to Damodaran, titled “Protein-polysaccharide hybrid hydrogels,” which is herein incorporated by reference.

A game component 113 may include one or more recognition element 345 configured to recognize an analyte 325, 340. A sensor may include one or more recognition element. One or more recognition element 345 may be immobilized or otherwise embedded in the game component 113, such as within one or more internal region 380. In some embodiments, a recognition element 345 may specifically bind an analyte 340. In some embodiments, a recognition element 345 may recognize one or more chemical substance. For example, a recognition element may include a peptide chain such as described in U.S. Pat. No. 7,402,423 to Taghizadeh, titled “Apparatus for the detection of pepsin,” which is herein incorporated by reference. A recognition element 345 may, for example, be in solution within or immobilized on a surface of an internal region 380. A recognition element 345 may include, for example, at least one cell, protein, polypeptide, nucleic acid, oligonucleotide, carbohydrate, lipid, conjugate, synthetic molecule, or mimetic. A recognition element 345 may be in a matrix 330, for instance conjugated to a matrix of agarose beads, or embedded or encapsulated within a matrix structure. A recognition element 345 might itself be a biologic, for example: a staphylococcus protein A complex, which generally binds immunoglobulins; a binding peptide or protein like an immunoglobulin; a DNA binding protein; a genetically engineered protein; a nucleic acid; an aptamer; a carbohydrate; a lipid; a conjugate; or a synthetic molecule like an artificial antibody or other mimetic. See, for example, U.S. Pat. No. 6,255,461 to Mosbach et al., titled “Artificial antibodies to corticosteroids prepared by molecular imprinting,” U.S. Pat. No. 5,804,563 to Still et al., titled “Synthetic receptors, libraries and uses thereof,” U.S. Pat. No. 6,797,522 to Still et al. titled “Synthetic receptors,” U.S. Pat. No. 5,831,012 to Nilsson et al., titled “Bacterial receptor structures” and U.S. Patent Application No. 2004/0018508 to Friedman, titled “Surrogate antibodies and methods of preparation and use thereof,” which are incorporated by reference. A recognition element 345 may include an antibody, such as an antibody saturated with a labeled form of the target, as described in U.S. Pat. No. 5,183,740 to Ligler et al., titled “Flow immunosensor method and apparatus,” which is herein incorporated by reference. In embodiments where glucose is an analyte to be optically detected by the external device, the recognition element may be a malachite green acceptor covalently linked to insulin. See, for example, Tolosa et al., “Lifetime-based sensing of glucose using energy transfer with a long lifetime donor,” Analytical Biochemistry 250: 102-108 (1997), which is herein incorporated by reference.

A game component 113 may include an encapsulating material. For example, in certain embodiments, a recognition element 335 might be encapsulated in one or more emulsion or other encapsulating material, or a coating, instead of or in addition to distribution throughout the game component 113 or within its internal region 380 and/or in the matrix 330 as illustrated in FIG. 3B. Proteins, for instance, have been shown to maintain their function when encapsulated. For more information regarding encapsulation of proteins, see, for example: “Fluorescence detection of enzymatic activity within a liposome based nano-biosensor,” Vamvakaki et al., Biosens Bioelectron. 21:384-8 (2005); Sotiropoulou, et al., “Stabilization of enzymes in nanoporous materials for biosensor applications,” Biosens Bioelectron 20:1674-1679 (2005); Besanger, et al., “Screening of inhibitors using enzymes entrapped in sol-gel-derived materials,” Anal. Chem. 75, 2382-2391, (2003), which are herein incorporated by reference. Emulsions and encapsulating materials can, for example, include one or more carbohydrate, alginate, protein, protein cage, lipid, phospholipid, liposome, cerasome, oil, emulsion, or a polymer. Encapsulating materials may include photopolymerized water-soluble molecules, such as those described in U.S. Pat. No. 6,258,870 to Hubbell et al., titled “Gels for encapsulation of biological materials,” which is herein incorporated by reference.

In certain embodiments, a game system 100 may include one or more biosensor. At least one sensor, wherein the at least one sensor is configured to detect one or more analyte obtained from the individual player, may include a biosensor. A biosensor may be incorporated within the principal game component 120 or the game component 113. For instance a biosensor could be included within the internal region 380 and/or be incorporated in the matrix 330 of the game component 113. As used herein, “biosensor” refers to a sensor including at least one biological agent or component. A biosensor may include cells, proteins, peptides, nucleic acids, aptamers, lipids, or carbohydrates. The biosensor might comprise in part a recognition element 345 such as a cell, a protein, a nucleic acid, an aptamer, a lipid, and/or a carbohydrate, configured to transmit a signal when a substance is detected. For example, a recognition element 345 may include one or more genetically engineered cells, which may be configured within solution or immobilized in alginate within the matrix 330. Such genetically engineered cells may be configured to detect a substance through a receptor and then to produce a bioluminescent signal. See, for example, Daunert et al., “Genetically Engineered Whole-Cell Sensing Systems: Coupling Biological Recognition with Reporter Genes,” Chem. Rev. 100(7): 2705-2738 (2000), which is herein incorporated by reference. As another example, the recognition element 345 may include an encapsulated enzyme configured to recognize an analyte 325, 340 as a substrate wherein the encapsulated enzyme is conjugated or otherwise associated with a responsive fluorescent compound. See, for example, Vamvakaki et al., “Florescence detection of enzymatic activity within a liposome based nano-biosensor,” Biosensors and Bioelectronics 21: 384-388 (2005), and Sotiropoulou, et al., “Stabilization of enzymes in nanoporous materials for biosensor applications,” Biosensors and Bioelectronics 20:1674-1679 (2005), and Besanger, et al., “Screening of inhibitors using enzymes entrapped in sol-gel-derived materials,” Anal. Chem. 75:2382-2391 (2003), which are herein incorporated by reference. As another example, one or more component of a biosensor may be a biologically active molecule bound to a surface, for example using gold binding fusion proteins. See, for example, the product description from BioHesion™ titled “Advanced Surface Binding Technology,” which is herein incorporated by reference. For example, a biosensor may include a bacterial protein. See “Scientists develop biosensor to detect E. Coli bacteria,” RxPG News, Aug. 19, 2006, which is herein incorporated by reference. For example, a biosensor may include human-derived antibodies and detect the presence of cells or cellular components. See Uchida et al., “A new assay using surface Plasmon resonance (SPR) to determine binding of the Lactobacillus acidophilus group to human colonic mucin,” Biosci. Biotechnol. Biochem. 68: 1004-1010 (2004), which is herein incorporated by reference.

In some embodiments, a game system 100 may include one or more taggant. For example, a game component 113 may include one or more taggant 350. For example a game component 113 may include one or more taggant 350 as part of the matrix 330. A taggant 350 may be configured to be responsive to a recognition element 345, such as a taggant 350 configured to be released when an analyte 340 binds to a recognition element 345. A taggant, as used herein, includes a chemical or physical component which is configured to be detectable, such as through direct visual or olfactory detection by a user, or detection through a device or assay. In some embodiments, a taggant 350 may be included in a matrix structure or retaining materials of a game component 113. In some embodiments, a game component 113 may be configured to store a taggant 350 at a distance from a matrix 330. A taggant storage region may be a reservoir. A taggant storage region may be configured to release a taggant at a specific time or in response to a condition, such as physical pressure, temperature, pH or hydration. For example, a taggant may be released through flexing of a support surface configured to be responsive to binding of a substance to recognition elements. See, for example, Boisen et al., “Rapid molecular detection of food- and water-bourne diseases,” Microbiology Today, August 2007, 116-118, which is herein incorporated by reference. Numerous types of taggants exist and various configurations may be utilized. A taggant 350 can include a dye, chromogen, a fluorescent substance, a luminescent substance, an odorant, a protein, a nucleic acid like an aptamer, a carbohydrate, a lipid, a synthetic molecule, a quantum dot, an optically active compound, a magnetic compound, a genetically engineered protein, a molecule configured for release, a resonance energy transfer molecule, a metal, a mass-label molecule, a radioisotope, or a volatile compound. For example, see U.S. Patent Application No. 2003/0022225 to Monforte et al., titled “Releasable nonvolatile mass label molecules,” U.S. Pat. No. 6,635,452 to Monforte et al. titled “Releasable nonvolatile mass label molecules,” U.S. Pat. Nos. 5,516,931, 5,604,104 and 5,602,273 to Giese et al., titled “Release tag compounds producing ketone signal groups,” U.S. Pat. No. 5,360,819 to Giese titled “Molecular analytical release tags and their use in chemical analysis,” and U.S. Pat. No. 6,491,643 to Katzman and Carlebach, titled “Breath test analyzer,” which are herein incorporated by reference. A taggant 350 may be included in a matrix 330 and released when a substance binds, such as in a displacement assay. The taggant 350 may be dehydrated prior to use, including dehydrated in complex with a recognition element 345. See, for example, U.S. Pat. No. 5,354,654 to Ligler et al., titled “Lyophilized ligand-receptor complexes for assays and sensors,” which is herein incorporated by reference. The taggant 350 may be a passive label for an analyte 325, 340, such as a nonspecific dye like a cyanine dye, configured to bind to nucleic acids. A taggant 350 may be configured to activate in the presence of the one or more analyte. For example, the taggant 350 may be configured to be responsive to binding of an analyte 325, 340, for example a labeled recognition element 345 like a fluorescein-conjugated antibody able to complex with an analyte 325, 340, or a recognition element 345 like a transferase that is configured to include a recognition site for an analyte 325, 340 and is configured to transfer the taggant 350 as a labeled modifier like a phosphate or carbohydrate group. See, for example, U.S. Patent Application No. 2003/0022225 to Monforte et al., titled “Releasable nonvolatile mass label molecules,” and U.S. Pat. No. 6,635,452 to Monforte et al. titled “Releasable nonvolatile mass label molecules,” which are herein incorporated by reference. If an analyte 325, 340 or a recognition element 345 includes a catalyst or enzyme, the taggant 350 may also include a substrate with a taggant configured to be cleavable or activatable. Another example includes a recognition element 345 configured to exhibit altered conformation upon binding an analyte 325, 340, such as a calcium-dependent binding molecule like calmodulin, possibly as part of a fusion protein, and/or configured to allow resonance transfer. See, for example, Miyawaki et al., “Fluorescent indicators for Ca²⁺ based on green fluorescent proteins and calmodulin,” Nature 388: 882-887 (1997), which is incorporated herein by reference. The taggant 350 may also be incorporated in or intrinsically part of one or more material forming the matrix 330 or a game component 113, and responsive to binding of an analyte 325, 340, such as a stimuli-responsive gel.

In some embodiments, a recognition element 345 may include a releasable taggant compound. Many types of releasable compounds are available, such as nonvolatile mass tags. See, for example, U.S. Patent Application No. 2003/0022225 to Monforte et al., titled “Releasable nonvolatile mass label molecules,” and U.S. Pat. No. 6,635,452 to Monforte et al. titled “Releasable nonvolatile mass label molecules,” which are herein incorporated by reference. Volatile release taggants may also be utilized in some embodiments. See, for example, U.S. Pat. No. 5,610,020 to Giese et al., titled “Release tag compounds producing ketone signal groups,” which is incorporated herein by reference. In some embodiments, a release taggant 350 may be presented in the matrix 330 as a lipid layer. See, for example, U.S. Pat. No. 6,949,347 to Singh and Chan-Hui, titled “Multiplex analysis using membrane-bound sensitizers,” which is herein incorporated by reference.

In certain embodiments, a game component 113 may include electronic circuitry, such as microcircuitry 365, and in some embodiments may include a power source 375 such as a microbattery, which may be housed, for instance, in the internal cavity 380 or within the outer wall 310 or in an additional region of the game component 113. A power source may include rechargeable or replacable power units. A power source may include wirelessly transmitted power sources, such as described in U.S. Patent Application No. 2005/0143787 to Boveja, titled “Method and system for providing electrical pulses for neuromodulation of vagus nerve(s), using rechargeable implanted pulse generator,” which is herein incorporated by reference. A game component 113 may include electronic circuitry, such as microcircuitry 365, and in some embodiments may include one or more lights 370, an antenna 360, and/or a buzzer 355. A game system 100 may include at least one antenna 360 operably attached to the at least one game component 113. A game component 113 including circuitry may be configured to be responsive to the presence of an analyte, for example a light or buzzer may be switched on in response to the detection of an analyte. A game component 113 including circuitry may be configured to be responsive to the presence of an analyte, for example a signal may be transmitted by an antenna 360 in response to the detection of an analyte.

The game system may also include additional sensors such as a thermometer or pH meter and/or instruments such as a timekeeping device or clock. The game system may include one or more sensing device such as a temperature sensor, pH detector, pressure sensor, or time-keeping device. In some embodiments, one or more taggant, medicinal agent, or signal may be operably connected to one or more sensing device, such as a taggant or medicinal agent reservoir which is triggered to release material at a preset time point. In some embodiments, a signal may be generated in response to one or more sensing device, such as a light or vibratory signal that is generated in response to the detection of a temperature, pH or pressure range. In some embodiments, data from one or more sensing device may be transmitted or recorded along with the sensed data, such as when temperature or pH relevant to the sensor is included in information communicated to a network.

The interior of a game component 113 may be configured as a single unit or as a plurality of regions, units, or modules, and may include layers of material. For example, a game component 113 may include layers, units, modules or regions of supporting materials, gels, matrices, or shell structures. FIG. 4 depicts various aspects of potential material configurations within a game component 113. For example, as illustrated in FIG. 4A, a game component 113 may include layers such as concentric layers, with an inner sphere 415 and an outer sphere 420. The layers may be enclosed by an outer wall 400. For example, as illustrated in FIG. 4B, a game component 113 may include regions, for example configured in sections 425. The sections 425 may be modules. For example, as illustrated in FIG. 4C, a game component 113 may include layers such as stratified layers 435, and may include one or more units configured as a scaffold. Multiple modular regions or layers may also form an indicator system for presence of a substance, such as described in the PCT patent application publication No. WO 2008/006152 A1 to Brockwell and Holland, titled “Indicator system for determining analyte concentration,” which is herein incorporated by reference.

In some embodiments, one or more recognition elements 345 may be configured in a region of a game component 113, such as the inner sphere 415 of a layered sphere, with an outer sphere 420 a selective medium of an appropriate material and configuration, such as a gel or membrane. In some embodiments, one or more recognition elements 345 may be configured in a region of the game component 113, such as within one or more stratified layer 435. In some embodiments, one or more encapsulated recognition elements 335 may be configured in a region of a game component 113, such as within one or more sections 425 or modules. A plurality of materials may be present throughout or in distinct regions of a game component, as shown in FIGS. 3A, 3B and 3C. In some embodiments, various regions may be configured from different materials, such as a different type of gel, like sol gels with varying pore size, or pH-responsive or ion-responsive gels. Embodiments with various regions configured from different materials would allow for the sensing of a variety of substances in different units of the discrete oral component. Embodiments with various regions configured from different materials would allow for ready identification of sensed analytes, such as by identification that module X senses analyte Y, and therefore if module X has sensed a substance, it is inferred to be analyte Y.

Referring now to FIG. 4C, in some embodiments, one or more gel or gel-like materials configured as part of a game component may include at least one recognition element 430 configured as one or more molecularly imprinted recognition site. See, for example, Byrne et al., “Molecular imprinting within hydrogels,” Advanced Drug Delivery Reviews 54: 149-161 (2002), Peppas and Huang, “Polymers and gels and molecular recognition agents,” Pharm Res. 19(5):578-87 (2002), and U.S. Patent Application No. 2007/0190084 to Hilt et al., titled “Polymer network compositions and associated methods,” which are herein incorporated by reference. In some embodiments, there may be a plurality of molecularly imprinted recognition sites associated with various recognition elements specific to particular regions. In some embodiments, a game component may include one or more gel configured to recognize and respond to an analyte 325, for example a hydrogel that selectively recognizes and sequesters a metal. See, for example, Peppas and Huang, ibid. and Tanaka et al., “Polymer gels that can recognize and recover molecules,” Faraday Discuss., 102: 201-206 (1996), which are herein incorporated by reference.

Other compounds could similarly be included in a game component 113 configured in an outer sphere 420, inner sphere 415, one or more sections 425, or one or more stratified layer 435. For example, regions of a game component may be configured to include one or more taggant 350. For example, materials may be included in a game component configured within an emulsion, in a coating, or may be incorporated into a structure such as a gel. In some embodiments, materials may be encapsulated 335, for example configured for release over time or configured for responsive release. A hydrogel may be configured for either slow release or responsive release of materials, depending on the embodiment. In some embodiments, materials may be retained in a reservoir within a game component 113, such as a reservoir configured to responsively release one or more medicinal agent. A game component 113 configured for placement in the oral cavity during game play activity may include at least one reservoir configured to release at least one medicinal agent. A reservoir may be configured to actively or passively release a medicinal agent. For example, a reservoir may include a slow-release gel. Compounds configured within a game component 113 may include, for example, one or more medicine like an expectorant, a bronchodilator, a cough suppressant, a vasodilator, an analgesic, an anti-septic, an anti-infective, an antibiotic, a nutritional supplement, or a therapeutic; a substrate for a metabolic enzyme; and/or a substance able to be physiologically incorporated as through skin absorption, ingestion, or inhalation. A substance able to be physiologically incorporated may include a diagnostic challenge like methacholine or an allergen, or may be an agent like dextrose or urea that is useful in testing the metabolic activity of the body or an infecting pathogen. See, for example, Pathak et al., ibid., which is incorporated herein by reference.

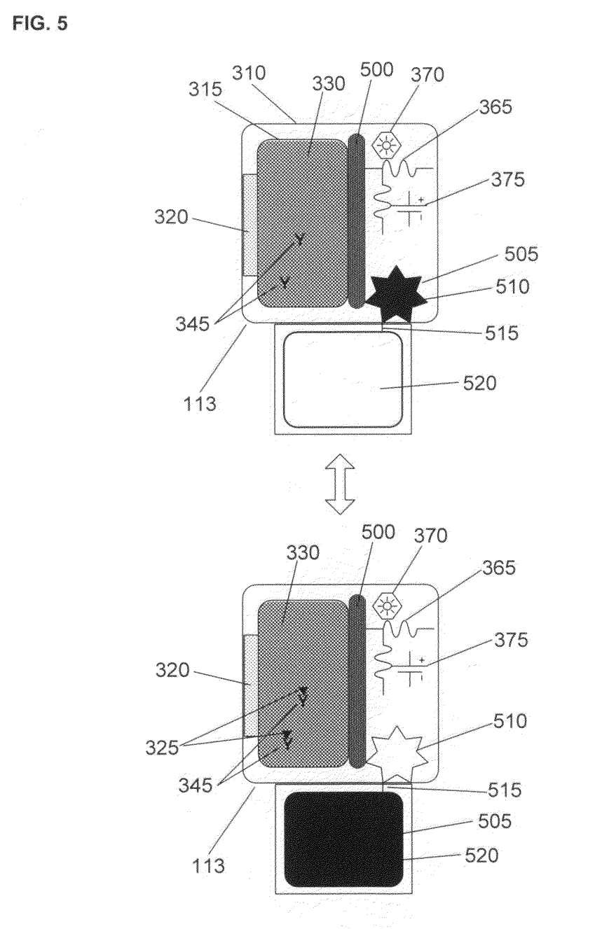

As illustrated in FIG. 5, in some embodiments a responsive gel may be configured as at least one sensor, which may be integral to a game component 113. For example, a responsive gel may be operably connected to a transducer configured to convert the response of the gel into a signal. FIG. 5 depicts a game component 113 in cross-section including an outer wall 310 and an inner wall 315. A selective medium 320 is configured between the interior region of the game component 113 and the exterior. The game component 113 may be configured to allow access to a responsive gel matrix 330 through a selective medium 320. For example, a selective medium 320 may be configured to allow access of an analyte 325 from the skin or salivary fluid of an individual game player into the interior of a game component 113. The interior of the game component 113 includes a responsive gel matrix 330 operably connected to a pressure sensor 500. The game component 113 depicted in FIG. 5 is configured so that the presence of an analyte 325 will elicit a response from the responsive gel matrix 330, such as swelling, light emission or release of a taggant, which may be detected by a detector. A responsive gel matrix 330 may include a swellable hydrogel operably connected to a signal output unit including a transducer, such as a pressure sensor 500 configured to convert the swelling response of the gel into a signal. See, for example, Bromberg, “Intelligent polyelectrolytes and gels in oral drug delivery,” Current Pharmaceutical Biotechnology 4: 339-349 (2003), which is herein incorporated by reference. A swellable hydrogel may include proteins such as the reversibly swellable, biodegradable, cation-binding hydrogel described in U.S. Pat. No. 6,310,105 to Damodaran, titled “Carboxyl-modified superabsorbent protein hydrogel,” which is herein incorporated by reference. In some embodiments, the swelling response of a gel may have stages responding to various ligands, which may be configured to be detectable by one or more transducers configured to respond to various stages of swelling. See, for example, Ehrick et al., “Genetically engineered protein in hydrogels tailors stimuli-responsive characteristics,” Nature Materials 4: 298-302 (2005), which is herein incorporated by reference. Examples of a transducer that may be configured for use in a signal output unit configured to respond to a responsive gel include a pressure sensor. A pressure sensor, for example, may be fabricated to include a piezoelectric material, such as an acoustical wave sensor or a cantilever sensor configured to convert the pressure of the gel into a sound, radiowave or wireless signal. See, for example: Drafts, “Acoustic Wave Technology Sensors,” Sensors Magazine Online, Oct. 1, 2000; Tanaka et al., ibid.; and Liu and Ji, “Detection of Pb²⁺ using a hydrogel swelling microcantilever sensor,” Analytical Sciences, 20:9-11 (2004), which are herein incorporated by reference. As depicted in FIG. 5, in some embodiments a pressure sensor 500 may be operably connected to circuitry 365, which may be configured to switch on a light 370 in response to the swelling of a responsive gel matrix 330. Circuitry 365 may include digital memory and a power source 375.

As depicted in FIG. 5, in some embodiments a game component 113 includes a signal output unit including an indicator material 505 stored in an indicator reservoir 510. An indicator reservoir 510 may be configured to release an indicator material 505 in response to a pressure sensor 500. An indicator reservoir 510 may be configured to release indicator material 505 into an indicator region 520, which may be a region configured to allow detection of the contents by a detection device or by inspection by an individual user. An indicator material 505 may include a material detectable by a detector or an individual user. For example, an indicator material 505 may include an ink or dye which is visibly detectable to an individual user in a visual inspection, such as through an optically permeable region, area, side or enclosure of the indicator region 520. For example, an indicator material 505 may include a chemiluminescent compound which is activated on release into the indicator region 520, the chemiluminescent signal from which is detectable by a detection device.

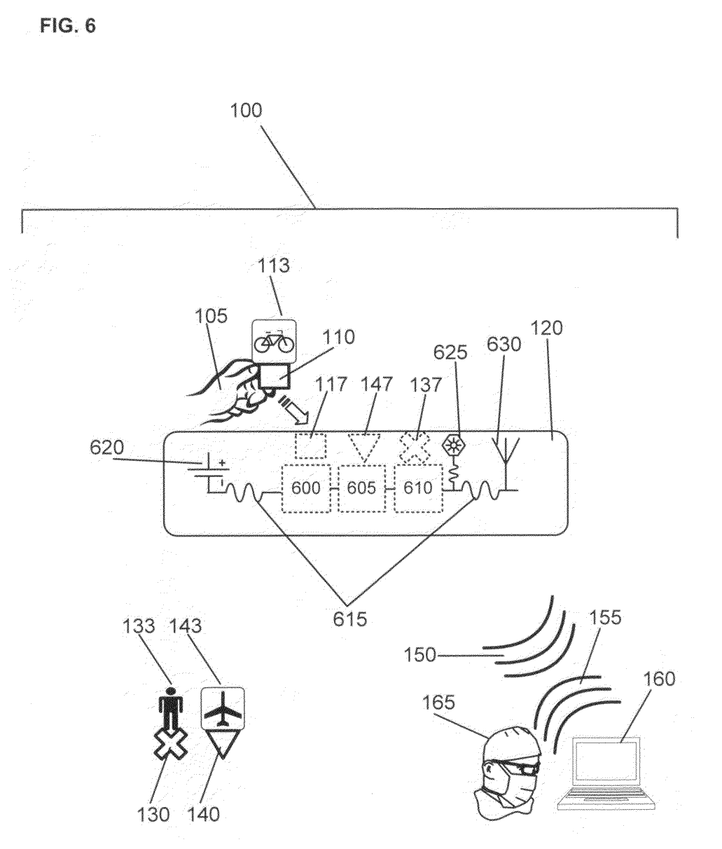

FIG. 6 depicts further aspects of a game system 100. FIG. 6 shows a principal game unit 120 in cross-section. As illustrated in FIG. 6, a principal game unit 120 may include one or more regions 117, 147, 137 configured to pair with one or more elements 110, 140, 130 of one or more game components 113, 133, 143. As illustrated in FIG. 6, one or more regions 117, 147, 137 configured to pair with one or more elements 110, 140, 130 of one or more game components 113, 133, 143 may be configured as indentations in the surface of the principal game unit 120, wherein the indentations are configured to mate with at least a portion of the one or more game components 113, 133, 143. Additionally or alternately, at least one principal game unit 120 may include a port for communication with the at least one game component 113, 133, 143. For example, a principal game unit 120 may include a port configured for the transmission of signals between the principal game unit 120 and a game component 113, 133, 143. For example, a principal game unit 120 may include a port configured for detection of signals from a sensor integral to a game component 113, 133, 143. FIG. 6 further illustrates that each of the one or more regions 117, 147, 137 of the principal game unit 120 configured to pair with one or more elements 110, 140, 130 of one or more game components 113, 133, 143 may be operably connected to one or more signal detectors 600, 605, 610. The signal detectors 600, 605, 610 are configured to detect a signal transmitted from a sensor system within the game components 113, 133, 143. A principal game component 120 may be configured to send a signal to a game component 113, 133, 143. A signal may include, for example, light, color changes, sound, vibration, infrared (IR), radio, wireless or other receivable signals. A signal from principal game component 120 may be part of the communication between a game component 113 and a network. For example, a principal game unit 120 and one or more game components 113, 133, 143 may be integrated with a system to provide light signals such as described in International Patent Application No. WO 99/31560 to Mueller et al., titled “Digitally controlled illumination methods and systems” which is herein incorporated by reference. A principal game component 120 may also include circuitry 615, and one or more of: a power source 620, an antenna 630, and a light 625. A light 625 and/or an antenna 630 may be configured to respond to the detection of a signal by a signal detector. A principal game unit may be configured to transmit to and receive signals 150, 155 from a network computing device 160. A network computing device 160 may indicate results or information to a system user 165 either in “real-time” or after a time delay. A principal game unit may include at least one signal transmitter configured to transmit a signal to at least one external device. A principal game unit may include at least one signal receiver configured to receive a signal from at least one external device.

A signal from at least one sensor may be part of the communication between a game component 113, 133, 143 and a principal game unit 120. For example, where a sensor is configured to emit light after binding one or more analyte, a principal game unit 120 may include a light detection device, such as a detection device configured to detect non-visible light or light of a specific wavelength. See, for example, U.S. Patent Application No. 2003/0143580 to Straus, titled “Rapid and sensitive detection of molecules,” which is herein incorporated by reference. In embodiments in which the game component 113, 133, 143 and/or an associated taggant is configured to emit optically-detectable signals, the one or more regions 117, 147, 137 of the principal game unit 120 may include in part or whole optically-permeable sections (e.g. windows), and a sensor or detector may include at least in part a spectrophotometer and/or light source configured to elicit signals from the game component or associated taggant. For example, a game component 113, 133, 143 or taggant may include at least one of a chromogen, fluorescent agent, luminescent agent, a quantum dot, or a compound configured to exhibit alterable optical density. A light source associated with a sensor system may include, for example, a light emitting diode or a white light source, such as a source configured to provide light in a variable and/or specific wavelength, including infrared (IR) or ultraviolet (UV). See, for example, U.S. Pat. No. 5,183,740 to Ligler et al., titled “Flow immunosensor method and apparatus,” U.S. Pat. No. 7,459,713 to Coates, titled “Integrated handheld sensing system approach for handheld spectral measurements having a disposable sample handling apparatus,” U.S. Patent Application No. 2008/0265146 to Coates, titled “Integrated sensing module for handheld spectral measurements,” and WIPO Patent Application Publication No. WO 2007/113727 to Kolesny-Chenko et al., titled “A portable food and/or beverage analyzer and a method of analyzing a food and/or beverage in the field,” which are herein incorporated by reference. For example, a light source may be configured to be a part of the detector that detects the opacity or colorimetric response of a component of the game component. See, for example: U.S. Pat. No. 6,623,698 to Kuo, titled “Saliva-monitoring biosensor head toothbrush;” U.S. Pat. No. 7,314,453 to Kuo, titled “Handheld diagnostic device with renewable biosensor;” U.S. Patent Application No. 2003/0023189 to Kuo, titled “Handheld diagnostic device with renewable biosensor;” and U.S. Patent Application No. 2002/0127143 to Kuo, titled “Saliva-monitoring biosensor electrical toothbrush,” which are herein incorporated by reference. In some embodiments, a principal game unit 120 may use electric pulses to measure the conductivity of a game component. See, for example, U.S. Pat. Nos. 6,623,698 and 7,314,453 to Kuo, ibid. In embodiments in which a taggant is a volatile compound or the analyte is in gaseous form, for example an oral or respiratory gas part of the salivary fluid, a sensor system may include a gas sensor such as an acoustic wave, chemiresistant, or piezoelectric sensor, such as those described as part of an “electronic nose.” See, for example, U.S. Pat. No. 5,571,401 to Lewis et al., titled “Sensor arrays for detecting analytes in fluids,” and U.S. Patent Application No. 2004/0006257 to Burch, titled “Detection, diagnosis, and monitoring of a medical condition or disease with artificial olfactometry,” which are herein incorporated by reference.

Depending on the embodiment, other sensor system types may include gas sensors, “electronic nose” sensors, “electronic tongue” sensors, nuclear magnetic resonance imagers, capillary electrophoretic devices, a volumetric sensor, or an optical sensor such as a spectrophotometer. See, for example: U.S. Pat. No. 5,303,585 to Lichte, titled “Fluid Volume Sensor;” Hagleitner et al., “Smart single-chip gas sensor microsystem,” Nature 414:293-296 (2001); Yusa et al., “Controlled multiple quantum coherences of nuclear spins in a nanometre-scale device,” Nature 434:1001-1005 (2005); U.S. Pat. No. 5,174,962 to Brennan titled “Apparatus for determining DNA sequences by mass spectrometry;” and Skelley et al., “Development and evaluation of a microdevice for amino acid biomarker detection and analysis on Mars,” Proc. Natl. Acad. Sci. USA, 102(4):1041-1046 (2005), which are herein incorporated by reference. See, for example, Lavigne et al., “Solution-based analysis of multiple analytes by a sensor array: toward the development of an “electronic tongue,” Journal of the American Chemical Society, 120: 6429-6430 (1998), which is herein incorporated by reference. A sensor may include a cantilever. Other examples of sensor systems may include technology such as optical microsensor arrays, surface enhanced raman spectroscopy (SERS), diode lasers, selected ion flow tubes, mass spectrometry, metal oxide sensors (MOS), infrared spectrometry, acoustic wave sensors, colorimetric tubes, infrared spectroscopy, conductive-polymer gas-sensors (chemoresistors), magnetic resonance, nanotechnology, and/or selective resonance techniques. See, for example, U.S. Patent Application No. 2007/0021458 to Ishikawa et al., titled “Selective resonance of bodily agents,” and Li et al., “The oral fluid MEMS/NEMS chip (OFMNC): diagnostic and translational applications,” Adv. Dent. Res., 18: 3-5 (2005), which are herein incorporated by reference.

A sensor integral to a game component may be configured to be directly responsive to an analyte. A sensor integral to a principal game unit may be configured to be directly responsive to an analyte. For example, a cantilever-based sensor may directly respond to the presence of an analyte. See Lee et al., “Chemical and biological sensor using an ultra-sensitive force transducer,” U.S. Pat. No. 5,807,758, which is herein incorporated by reference. Instead or in addition, a sensor may be configured to be indirectly responsive to at least one of the one or more analyte. For example, at least one sensor may be configured to be responsive to a metabolite of at least one of the one or more analyte. For example, at least one sensor may be configured to be responsive to at least one taggant. For example, at least one sensor may be configured to be responsive to a metabolic byproduct of an analyte.

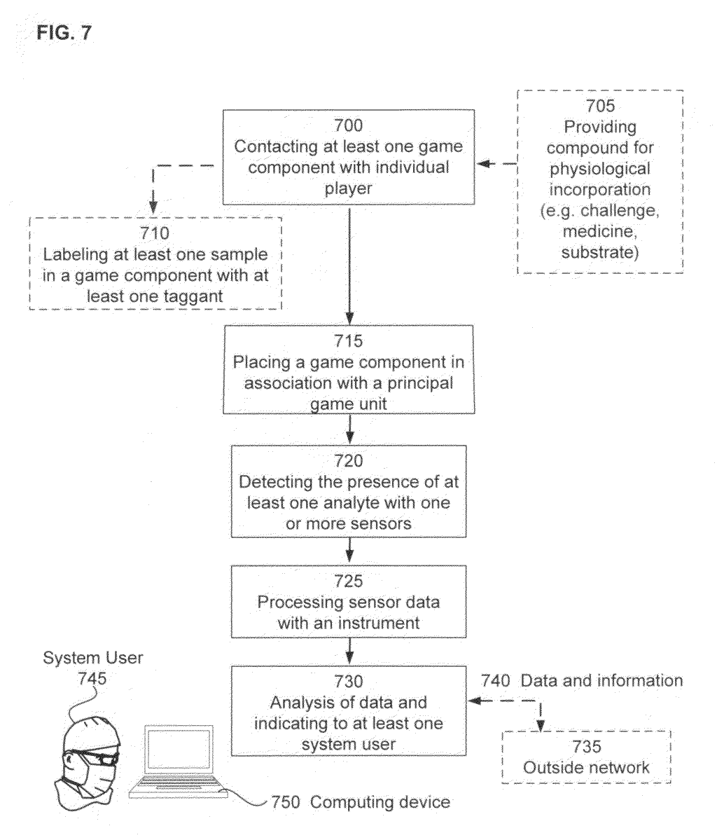

FIG. 7 depicts a flowchart illustrating aspects of a method of detecting at least one analyte with a game system such as those described herein. Box 700 illustrates contacting at least one game component with an individual player. For example, a game component may come into contact with an individual player during routine game play, including contact with an individual game player's hand, arm, mouth, or other body part. Multiple game components may come into contact with a single player. Each game component comes into contact with a single game player. Multiple players may come into contact with at least one game component per player, and the game components used by different players may have distinct components, such as matrix or sensor modules. Box 705 shows an optional step of providing at least one compound for physiological incorporation. For example, at least one challenge compound, medicine, or substrate may be provided as part of the game system. For example, at least one challenge compound, medicine, or substrate may be provided with a covering of a game component, such as an emulsion or dried coating. Optional block 710 depicts labeling at least one sample in a game component with at least one taggant. For example, a taggant may be incorporated into the interior of a game component. Block 715 illustrates placing a game component in association with a principal game unit. For example, a game component may be placed in a region of the principal game unit configured to mate with the game component, such as an indentation. For example, a game component may be placed in a region of the principal game unit marked as a “goal,” “score” or similar region. Block 720 shows detecting the presence of at least one analyte with one or more sensors. For example, at least one analyte may be transferred from an individual game player to a game component, which includes one or more sensors configured to respond to the at least one analyte. Block 725 depicts processing sensor data with an instrument. For example, in embodiments where a sensor emits light in the presence of an analyte, the light may be detected with a light detector and the resulting data processed on a microprocessor. Block 730 illustrates analysis of data and indicating to at least one system user. For example, the system may analyze the data and then indicate to a system user 745 that the analysis is complete. For example, the data from the sensor may be analyzed, such as in combination with similar data from the same individual game player from an earlier time point, and then a result indicated to a system user 745. Data and information 740 may be transmitted to and received from an outside network 735, including to and from one or more computing device 750.

Data and information from the systems and methods utilizing game systems may be used in correlations between other types of data (for example, analyte standards based on blood serum or urine analysis). Data and information from the systems and methods utilizing game systems may be integrated with other types of information, such as standards or ranges, relevant for standard clinical parameters. For example, data indicating the range of antibodies detected may be compared to a standard range from a population. For example, data indicating physiological parameters such as pulse and blood pressure may be integrated with standard clinical ranges of those values. Data and information from the systems and methods utilizing game systems may be integrated with previously-recorded, or simultaneously-recorded, data from the same patient. For example, the presence or absence of analytes relating to bacterial infection from a single individual may be integrated over time to result in a temporal view of the clinical progress of an infection. For example, data relating to the metabolization of a drug may be integrated from multiple times to result in an average or mean metabolic concentration for a specific individual. Data and information, including correlations and integrated information, may be stored in a local device or a remote device, such as a network device or a medical data system. Data and information, including correlations and integrated information, may be indicated to a system user such as medical personnel or caregivers immediately or at a later time. For example, if data describing a clinically-relevant analyte from an individual player is determined by the system to be outside of a preset range, an alarm or indication may be given immediately to alert medical personnel or caregivers.

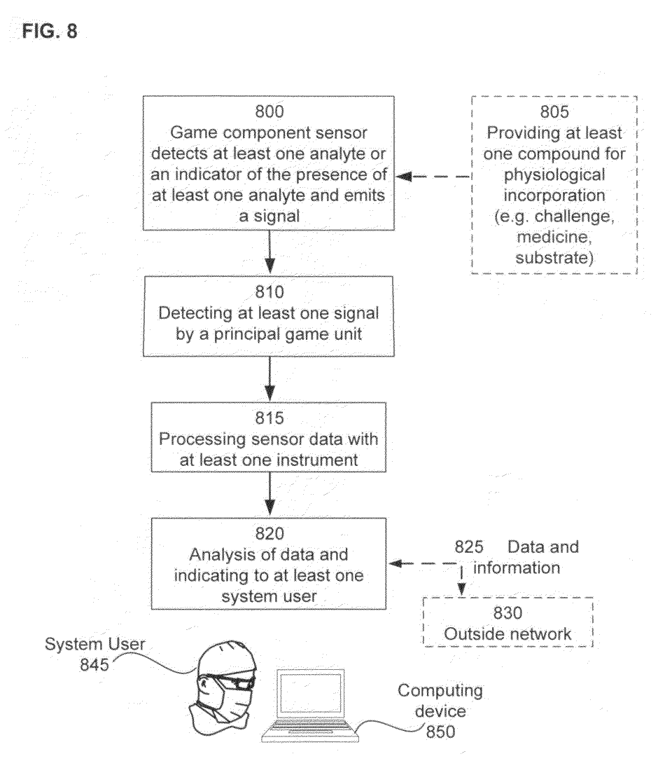

FIG. 8 depicts a flowchart illustrating aspects of the systems and methods described herein. Block 800 shows that a game component sensor detects at least one analyte, or an indicator of the presence of at least one analyte, and emits a signal. For example, a game component may include a matrix incorporating a responsively-swelling gel matrix, which is configured to press on a pressure sensor when an analyte is present. Circuitry operably attached to the pressure sensor may be configured to transmit a signal, such as emission of infrared (IR) in response to the detection of the swelling gel matrix. Optional block 805 depicts that the method may include providing at least one compound for physiological incorporation, for example a challenge, a medicine, or a substrate configured for physiological incorporation by at least one individual game player. Block 810 shows detecting at least one signal by a principal game unit. For example, wherein a game component sensor emits a gas in the presence of an analyte, a principal game unit may detect the presence of the gas as a signal. For example, wherein a game component sensor is configured with circuitry that emits an IR signal in the presence of an analyte, a principal game unit may detect the presence of the IR signal. Block 815 illustrates processing sensor data with at least one instrument. For example, wherein the game component sensor emits IR in response to the presence of an analyte, the instrument may be an IR detector which detects the IR and converts the signal into digital information. Block 820 shows analysis of data and indicating to at least one system user. For example, a computing device 850 may implement an analysis of the data, such as integrating it with other data regarding the individual game player, and then indicate to at least one system user 845 the data analysis. For example, a computing device 850 may implement an analysis of the data, such as integrating it with other relevant medical information such as standard toxic analyte levels or previously-known therapeutically effective levels, and then indicate the analysis to a system user 845, for example on the display of a computing device 850. Data and information 825 relevant to the analysis may also be transmitted to and from an outside network 830, which may include a computing device 850.

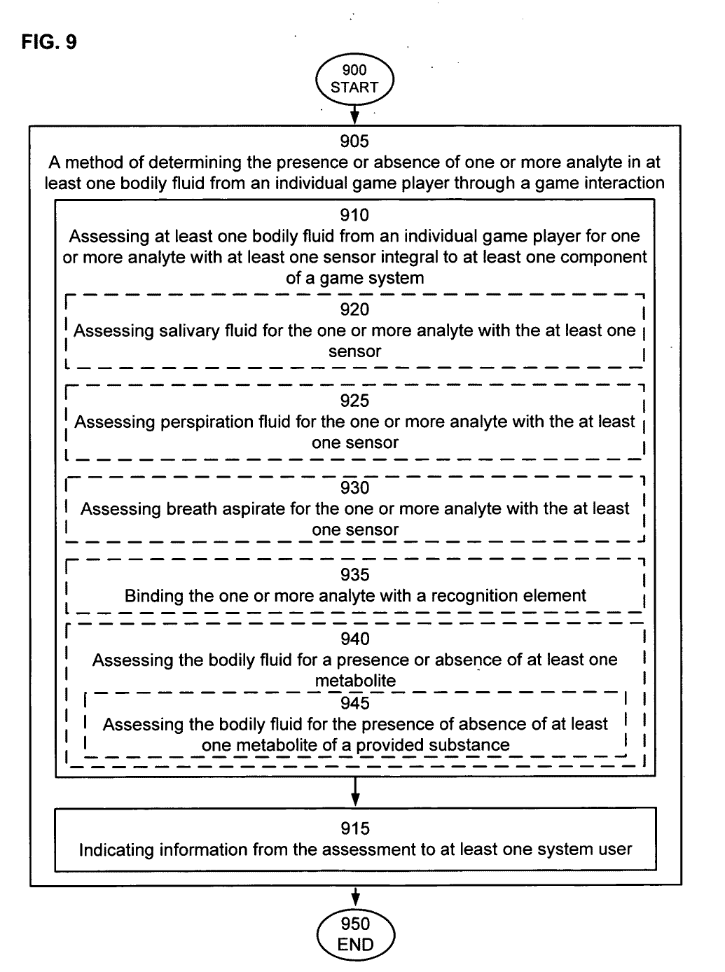

FIG. 9 illustrates a flowchart outlining further aspects of the systems and methods described herein. Block 900 shows the start of a method. Block 905 depicts that the method is a method of determining the presence or absence of one or more analyte in at least one bodily fluid from an individual game player through a game interaction. The method flowchart includes block 910, illustrating assessing at least one bodily fluid from an individual game player for one or more analyte with at least one sensor integral to at least one component of a game system. For example, salivary fluid from at least one individual game player may be assessed with sensor integral to an individual game component. For example, perspiration fluid may be assessed with a sensor integral to a principal game unit. The method flowchart includes block 915, showing indicating information from the assessment to at least one system user. For example, the method may include indicating information on a computing device through a display screen, or an auditory indication. For example, the method may include indicating information on a display operably attached to a principal game unit. Block 910 may include one or more of blocks 920, 925, 930, 935 and 940. Block 940 may include block 945. Block 920 depicts assessing salivary fluid for the one or more analyte with the at least one sensor. For example, a sensor may be incorporated with a game component that is configured as a candy item, including a flavorant coating, and an individual game player may be encouraged to suck on the game component during game play activity, thus transferring salivary fluid to the game component. Block 925 shows assessing perspiration fluid for the one or more analyte with the at least one sensor. For example, caffeine in perspiration fluid may be detected with the at least one sensor integral to a game component configured to be worn by an individual player, such as including a sweatband or undershirt. Block 930 illustrates assessing breath aspirate for the one or more analyte with the at least one sensor. For example, a principal game unit may include a sensor configured to detect ethanol levels in breath aspirate and a breathing tube operably attached to the sensor. During game play activity, a game player may be encouraged to breathe into the tube and therefore transfer breath aspirate into the principal game component. Block 935 depicts binding the one or more analyte with a recognition element. For example, a recognition element may be incorporated into a matrix within a sensor. Block 940 shows assessing the bodily fluid for a presence or absence of at least one metabolite. Block 940 may include block 945, illustrating assessing the bodily fluid for the presence or absence of at least one metabolite of a provided substance. Block 950 illustrates the end of the method.

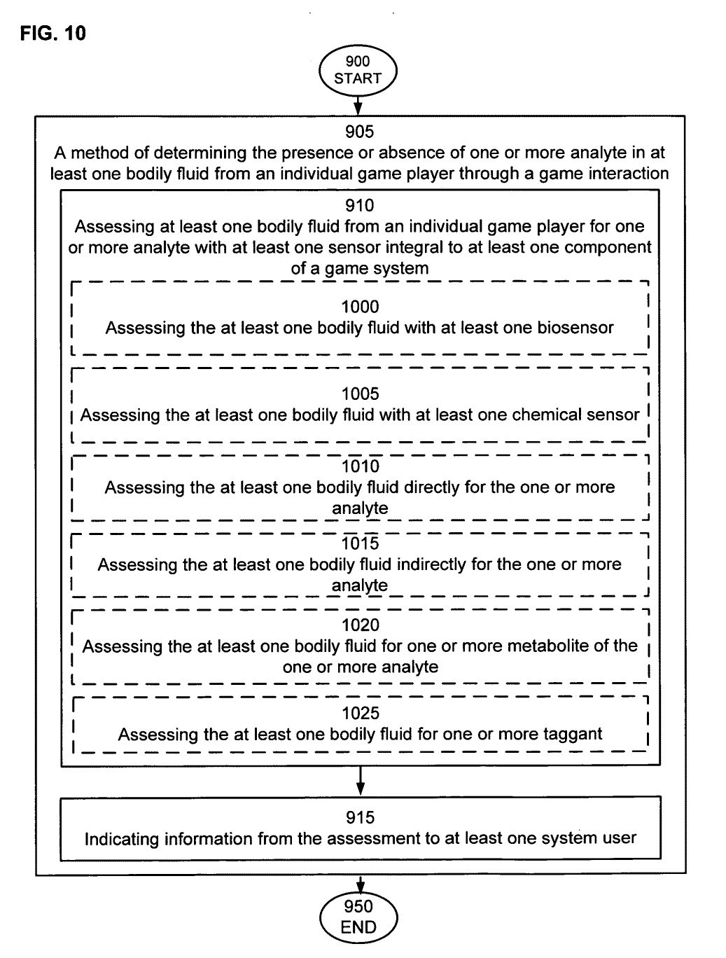

FIG. 10 illustrates other aspects of the method flowchart shown in FIG. 9. FIG. 10 shows that block 910, which depicts assessing at least one bodily fluid from an individual game player for one or more analyte with at least one sensor integral to at least one component of a game system, may include one or more of blocks 1000, 1005, 1010, 1015, 1020 and 1025. Block 1000 depicts assessing the at least one bodily fluid with at least one biosensor. For example, a sensor in a game system may include at least one protein component which originated from a cell as an active component of the sensor. Block 1005 shows assessing the at least one bodily fluid with at least one chemical sensor. For example, a sensor integral to a game system may include an “electronic tongue” component. See, for example, Lavigne et al., “Solution-based analysis of multiple analytes by a sensor array: toward the development of an “electronic tongue,” Journal of the Americal Chemical Society, 120: 6429-6430 (1998), which is herein incorporated by reference. Block 1010 depicts assessing the at least one bodily fluid directly for the one or more analyte. For example, a bodily fluid may include an analyte that binds directly with a component of a sensor, such as a recognition element. Block 1015 illustrates assessing the at least one bodily fluid indirectly for the one or more analyte. For example, a cofactor, associated element, or marker of an analyte may be assessed in bodily fluid, such as a pathogen-associated protein which may serve as an indirect marker of the presence of the entire pathogen and associated toxins. For example, enteropathogenic E. coli express a cell surface protein, intimin, which may be used to indirectly detect enteropathogenic E. coli as a whole. See Horner et al., “A proteomic biosensor for enteropathogenic E. coli,” Biosensors and Bioelectronics, 21: 1659-1663 (2006) and U.S. Pat. No. 7,292,349 to Miller, titled “Method for biomolecular sensing and system thereof,” which are herein incorporated by reference. Block 1020 depicts assessing the at least one bodily fluid for one or more metabolite of the one or more analyte. For example, wherein the analyte is methamphetamine, the metabolite of the analyte may be amphetamine. A game system may assess perspiration fluid for the presence or absence of amphetamine. See Barnes et al., “Excretion of methamphetamine and amphetamine in human sweat following controlled oral methamphetamine administration,” Clinical Chemistry 54: 172-180 (2008), which is herein incorporated by reference. Block 1025 illustrates assessing the at least one bodily fluid for one or more taggant. For example, a signal output unit may be configured to react with a specific chemical taggant released in response to binding of an analyte to the sensor.

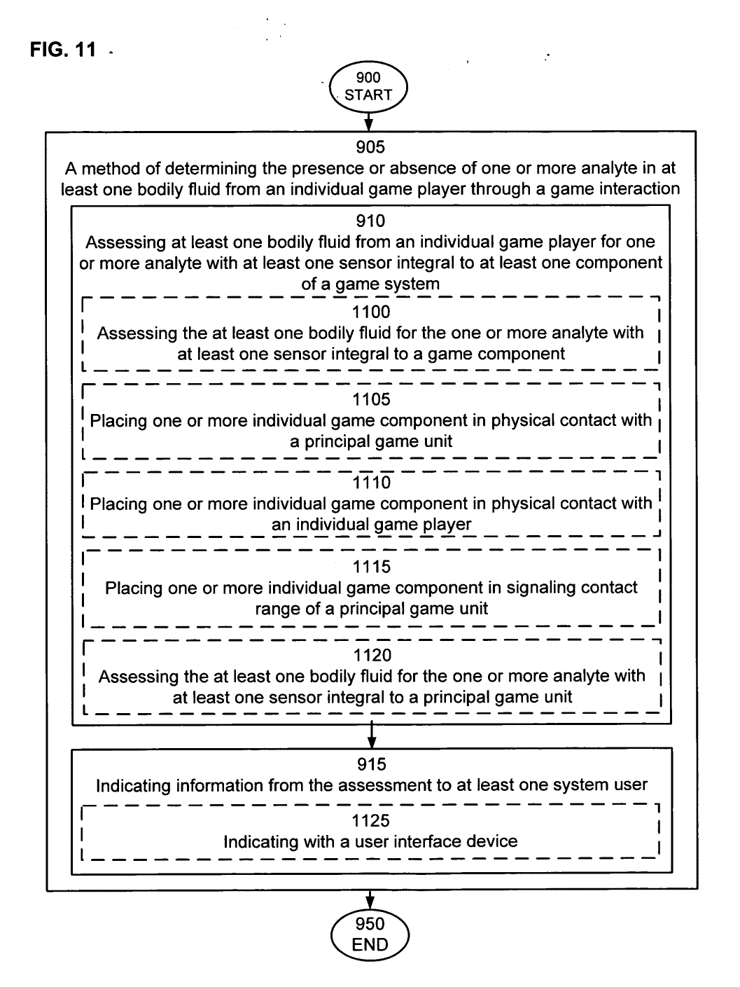

FIG. 11 illustrates further aspects of the method flowchart shown in FIG. 9. Block 910, depicting assessing at least one bodily fluid from an individual game player for one or more analyte with at least one sensor integral to at least one component of a game system, may include one or more of blocks 1100, 1105, 1110 1115 and 1120. Block 915, showing indicating information from the assessment to at least one system user, may include block 1125. Block 1100 shows assessing the at least one bodily fluid for at least one analyte with at least one sensor integral to a game component. For example, a sensor may be integral to a game component. For example, a detector may be operably attached to a port in the principal game unit, such as a window configured to allow a relevant IR signal to pass to the detector from a signal output unit operably attached to a sensor integral to a game component. Block 1105 depicts placing one or more individual game component in physical contact with a principal game unit. For example, a game component may be placed in physical contact with a region of the principal game unit during the game activity. For example, a game component may be placed in physical contact with a port configured for communication of the principal game unit during the game activity. Such placement may align the game component and the principal game unit to assist in communication between the game component and the principal game unit. Block 1110 shows placing one or more individual game component in physical contact with an individual game player. For example, a game component may be strapped on to the arm or wrist of a game player, or held in the hand of an individual game player. For example, a game player may wear a game component configured like a glove or hand covering during game play activity. Block 1115 illustrates placing one or more individual game component in signaling contact range of a principal game unit. For example, an individual game component may be placed in RF signaling contact range as part of game activity. For example, an individual game component may be placed in IR signaling range when an individual game component is placed in an indentation in the principal game unit, such as during game activity. Block 1120 depicts assessing the at least one bodily fluid for the one or more analyte with at least one sensor integral to a principal game unit. Block 915, showing indicating information from the assessment to at least one system user, may include block 1125. Block 1125 depicts indicating with a user interface device. For example, a system may be configured to automatically display, on assessment of the presence or absence of an analyte, a detection result, or the existence of an assessment result, on a display operably connected to a computing device. For example, a system may be configured to automatically indicate, with an audible tone, the presence of an analyte upon detection of an analyte.

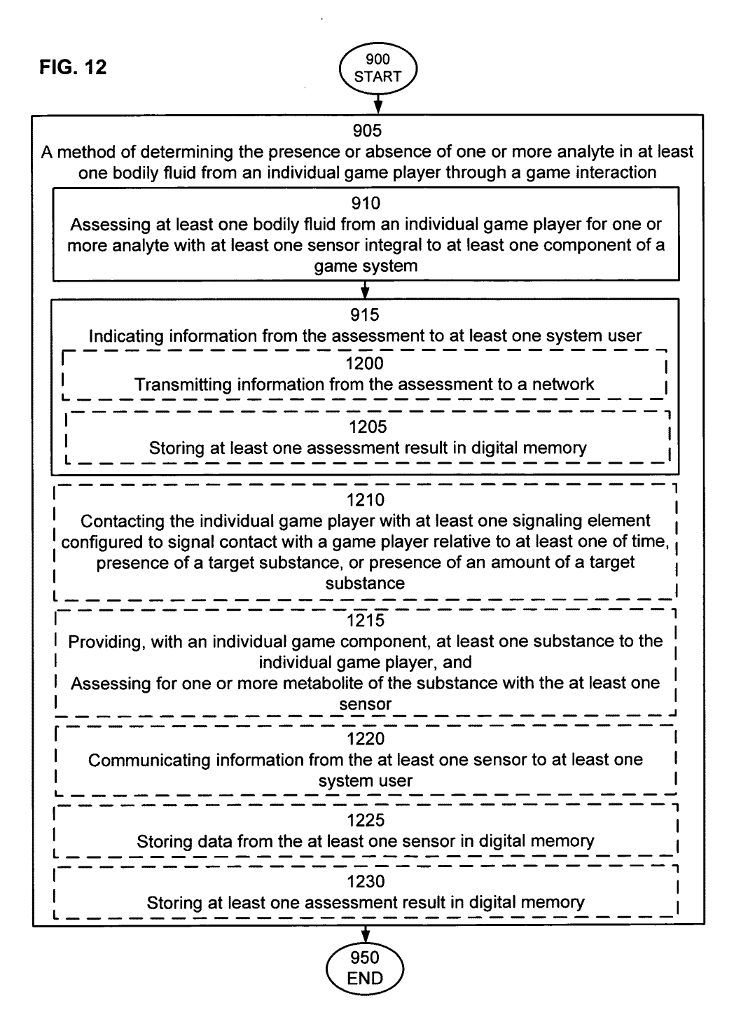

FIG. 12 depicts further aspects of the method flowchart shown in FIG. 9. Block 915, depicting indicating information from the assessment to at least one system user, may include one or more of blocks 1200 and 1205. The flowchart may also include at least one of blocks 1210, 1215, 1220, 1225 and 1230. Block 1200 illustrates transmitting information from the detection to a network. For example, information relating to the detection of a specific analyte or group of analytes, the date, time, or analyte levels may be transmitted from a detection device incorporated in a principal game unit to a network. For example, information may be transmitted wirelessly, such as through RF signals, to a network. For example, information may be transmitted via a conduit, such as a wire, to a network. Block 1205 depicts storing at least one assessment result in digital memory. For example, information from at least one assessment may be stored in digital memory, wherein the digital memory unit is physically located in a game component, a principal game unit, or a remote computing device. Block 1210 shows contacting the individual game player with at least one signaling element configured to signal contact with a game player relative to at least one of time, presence of a target substance, or presence of an amount of a target substance. For example, a pressure sensor integral to a game component may be operably connected to an indicator light in the game component, so that when an individual game player holds the game component for a minimum length of time a light indicator is switched on. For example, a fluid sensor integral to a game component including flavorant and configured to be held in the oral cavity of a game player may be operably attached to a RF signal emitter configured to transmit an RF signal when a sufficient quantity of salivary fluid has been detected. A signaling element may indicate, for example, to a system user or individual game player that the system is operational. Block 1215 depicts providing, with an individual game component, at least one substance to the individual player, and detecting one or more metabolite of the substance with the at least one sensor. For example, urea containing a nonstandard carbon isotope may be provided to an individual player and the resulting nonstandard isotope-containing carbon dioxide may be detected with a sensor. See Pathak et al., ibid. Block 1220 shows communicating information from the at least one sensor to at least one system user. For example, the presence of an analyte may be communicated via the system to a system user and visualized on a display. For example, a signaling element may be operably attached to a light and the system configured to switch on the light to indicate to a system user that the system is operational. Block 1225 illustrates storing data from the at least one sensor in digital memory. For example, data from a sensor indicating the time or date may be recorded in digital memory. Block 1230 indicates storing at least one assessment result in digital memory. For example, information relating to the assessment of the presence or absence of an analyte may be saved in digital memory, such as the date, time, individual game player, level of analyte detected, and specific game component(s) used in the detection. Saving at least one assessment result in digital memory may be implemented in a principal game component, in a computing device, and may occur in a network. For example, data relating to an analyte detection, such as the presence or absence of an analyte, may be recorded in digital memory. A digital memory unit may be physically located, for example, in a game component, a principal game unit, or a remote computing device.

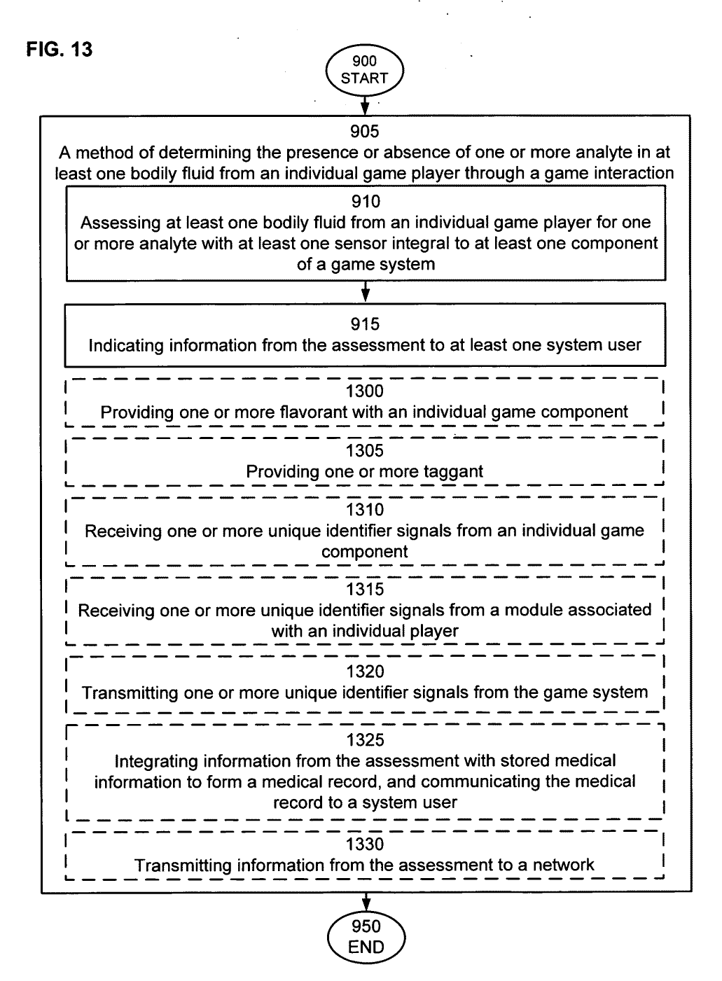

FIG. 13 illustrates further aspects of the method flowchart shown in FIG. 9. The flowchart may include one or more of blocks 1300, 1305, 1310, 1315, 1320, 1325 and 1330. Block 1300 shows providing one or more flavorant with an individual game component. For example, a flavorant may be incorporated on the exterior of the game component as a coating. For example, a flavorant may be incorporated within a reservoir of the game component configured for active or passive release. Block 1305 depicts providing one or more taggant. For example, a taggant may be provided with a game component, such as incorporated within a matrix. Block 1310 illustrates receiving one or more unique identifier signals from an individual game component. For example, a principal game component may be configured to receive RFID signals from a group of game components and the system may individually identify specific game components by associated RFID signals specific to each game component. Block 1315 depicts receiving one or more unique identifier signals from a module associated with an individual player. For example, an individual player may carry a game module configured to transmit a unique RFID code and the game system configured to respond to that code, such as by changing the game play, switching on a particular sensor and/or detector, or transmitting related information to a network. A module carried by an individual player may be integrated with a game component or it may be a separate unit, such as an ID ring or bracelet. Block 1320 depicts transmitting one or more unique identifier signals from the game system. For example, wherein multiple game systems are in communication with a network, each individual game system may transmit a unique identifier signal to identify its transmitted data specifically to the network. Block 1325 illustrates integrating information from the assessment with stored medical information to form a medical record, and communicating the medical record to a system user. For example, information from the detection may be integrated with stored medical information such as a game player's medical diagnosis, current approved medications, and known allergies to form a medical record which may be communicated to a system user through a computing device or a network. Block 1330 shows transmitting information from the assessment to a network. For example, a game system may transmit information to a network that the game system is operational, or that it requires maintenance, and an assessment is unreliable. For example, a game system may transmit information to a network including the results of an assessment, the presence or absence of an analyte, or a physiological parameter (e.g. blood pressure, body temperature or pulse rate of an individual game player).

Other aspects of the systems and methods described herein are described in the examples below.

### EXAMPLES

### Example 1

### Game System to Detect Antibodies to Viral Pathogens in Saliva and Indicate their Presence to Individual Users and Healthcare Workers and Caregivers

A game system is described herein that is configured to detect antibodies (e.g., IgG, IgM, IgA) to viral pathogens in the salivary fluid of individuals playing a game on the game system and to indicate the presence of the antibodies to individual game players, caregivers, family members and healthcare workers. The game system may be used to monitor the health or disease status of individuals, including monitoring the immunization status of individuals who have been vaccinated for microbial pathogens. The game system indicates the health status and/or immunization status of the individual to a network that may be accessed by the individual player, parents, teachers, nurses, caregivers, and public health workers.

Individual game components include unique radiofrequency identification (RFID) tags that are assigned to each individual game player (i.e. child, parent, student, patient, teacher, health care professional) by scanning the game component containing an RFID tag over the principal game unit containing an RFID reader module. Game components uniquely identified by RFID tags may be discarded after playing the game, or game components may be modular, with a RFID tag subsequently transferred to a fresh game component so that each player retains their unique RFID tag. Alternatively, a reusable principal game unit may include a RFID reader module and each individual may be assigned a unique RFID tag incorporated in a game module such as a bracelet or neck tag. RFID tags and reader modules are available from GAO RFID Inc., (Seattle, Wash.).

Game components include a semi-permeable selective medium covering an opening to the interior of the game component that allows salivary fluid and its contents to pass into the interior but excludes macroscopic particles, food and other constituents that might interfere with analysis of the salivary fluid and its contents. For example, devices to collect salivary fluid are described in U.S. Pat. No. 6,022,326 to Tatum et al., titled “Device and method for automatic collection of whole saliva,” which is incorporated herein by reference. A selective medium is manufactured from polyethylene. The individual game components are configured to be placed in the mouth and include a sugar-based coating incorporating flavorant to encourage individual players to hold the individual game components in their mouths.

When an individual has been infected or immunized with a specific virus, salivary fluid may contain antibodies that bind to viral antigens. For example, one may detect IgM antibodies in salivary fluid that recognize Dengue virus antigens. Dengue virus infection results in the presence of anti-Dengue virus IgM in salivary fluid, as shown by Balmaseda et al., “Diagnosis of Dengue virus infection by detection of specific immunoglobulin M (IgM) and IgA antibodies in serum and saliva,” Clin. Diag. Lab. Immun., 10: 317-322 (2003) which is incorporated herein by reference. Moreover, analysis of salivary fluid from individuals immunized with an influenza virus vaccine may detect IgG antibodies that recognize influenza virus. Vaccination with an inactivated influenza virus vaccine derived from influenza-A/Chile/83 (H₁N₁), -A/Mississippi/85 (H₃N₂) and -B/Ann Arbor/1/86 (influenza vaccines are available from Sanofi Pasteur, Lyon, France) results in the appearance in saliva and blood of anti-influenza IgG antibodies which can be detected in salivary fluid 13 days after vaccination as shown by Moldoveanu et al., “Human immune responses to influenza virus vaccines administered by systemic or mucosal routes,” Vaccine, 13: 1006-1012 (1995), which is incorporated herein by reference.

Salivary fluid may also be used to determine immunization to and/or infection with measles virus, mumps virus and rubella virus. Assays of anti-viral antibodies in salivary fluid following infection or immunization indicates the presence of antibodies in salivary fluids that correlates with serum levels, with sensitivities and specificities of 97% and 100% for measles, 94% and 94% for mumps, and 98% and 98% for rubella, respectively. See Thieme et al., “Determination of measles, mumps and rubella immunization status using oral fluid samples,” JAMA 272: 219-221 (1994), which is incorporated herein by reference.

A game component configured for the detection of viral antibodies in salivary fluid includes an antigen microarray with multiple viral antigens imprinted on the microarray to recognize and capture anti-viral antibodies derived from salivary fluid that come into contact with microarray. Microarrays are fabricated by printing viral proteins on silanized glass slides (available from CEL Associates, Inc. Pearland, Tex.) using a computer-controlled high-speed robotics system described in Schena et al., “Quantitative monitoring of gene expression patterns with a complementary DNA microarray,” Science, 270: 467-470 (1995) and Mezzasoma et al., “Antigen microarrays for serodiagnosis of infectious diseases clinical chemistry,” Clin. Chem. 48: 121-130 (2002) which are incorporated herein by reference. For example, viral antigens from cytomegalovirus (CMV), herpes simplex virus (HSV) types 1 and 2 and rubella virus, measles virus, mumps virus, respiratory syncytial virus, Epstein-Barr virus, hepatitis A virus, hepatitis B virus, hepatitis C virus and human immunodeficiency virus type 1 (HIV-1) are available from Meridian Life Science, Inc. (Saco, Me.), and they may be imprinted using stainless steel solid pins 200 μm in diameter that transfer approximately 1 nanoliter of antigen solution to a slide. See Mezzasoma et al., “Antigen microarrays for serodiagnosis of infectious diseases clinical chemistry,” Clin. Chem. 48: 121-130 (2002), which is incorporated herein by reference. A viral antigen microarray within a game component may be connected to an opening that collects salivary fluid from an individual playing the game. The viral antigen microarray may recognize and bind to anti-viral antibodies present in the salivary fluid thus capturing the anti-viral antibodies. For example, antigen microarrays may capture anti-viral antibodies recognizing CMV, HSV and rubella virus as shown by Mezzasoma et al., ibid.

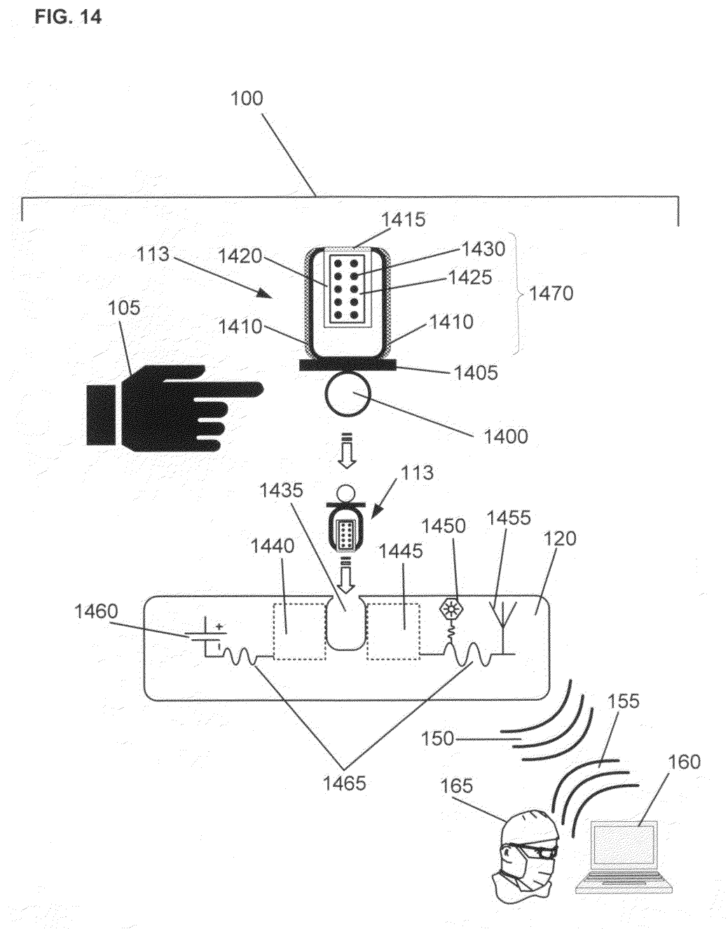

As an illustration, FIG. 14 depicts a game system 100 including a game component 113 including a ring 1400 configured for an individual game player 105 to hold the game component 113, such as by wearing the game component 113 on a finger or wrist. The game component 113 includes a base 1405 between the ring 1400 and a sensor region 1470. The game component 113 and a principal game unit 120 are depicted in FIG. 14 in cross-section to illustrate interior aspects. The game component 113 includes a selective membrane 1415 between the exterior of the game component 113 and an interior salivary fluid collection chamber 1420. The selective membrane 1415 is fabricated from polyethylene configured to allow salivary fluid from the individual game player 105 to enter the interior salivary fluid collection chamber 1420 and exclude large particulates, such as food residue or cellular debris. The interior salivary fluid collection chamber 1420 may be fabricated to have negative pressure prior to use to encourage the collection of salivary fluid in the chamber. The interior salivary fluid collection chamber 1420 includes a viral antigen microarray 1425 including multiple regions with imprinted viral antigen “spots” 1430. The exterior of the sensor region 1470 is coated with a candy coating 1410, including flavorant, wherein the candy coating 1410 is configured to coat the surface of the sensor region 1470 but allow access of salivary fluid to the selective membrane 1415.

A game module containing a viral antigen microarray 1425 with anti-viral antibodies bound to it 1430 may be placed in a principal game unit 120 that is configured to process and analyze the bound antibodies. A principal game unit containing a mini-pump and solenoid valves (both are available from Parker-Hannifin, Precision Fluidics Division, Hollis, N.H.) and reservoirs may dispense a wash buffer, 2× phosphate buffered saline (PBS) containing 10 g/L Tween 20. (1×PBS contains: 0.2 g/L KCl, 1.44 g/LNa2HPO4, 0.24 g/L KH2PO4, 8 g/L NaCl, pH 7.4.) to wash the microarray free of salivary fluid and loosely bound proteins. The principal game unit also contains fluorescently-labeled anti-human IgG, anti-human IgM and anti-human IgA antibodies. Anti-human antibodies are available from Sigma-Aldrich Co., (St. Louis, Mo.) and they may be labeled with fluorescent molecules, fluors (e.g. AlexFluor546™ and AlexaFluor594™), using fluors and kits available from Molecular Probes-Invitrogen, (Carlsbad, Calif.). Fluorescent antibodies are delivered to and incubated with the microarray containing bound anti-viral antibodies, and the microarray is washed by delivery of wash buffer. Detailed descriptions and protocols for using antigen microarrays are given in Mezzasoma et al., ibid. Analysis of the microarrays is done by scanning confocal microscopy with a moving magnet scanner (available from Cambridge Technology, Inc., Lexington, Mass.) contained in the principal game unit. Images are generated with ScanDesign™ software made by GSI Lumonics, GSIG, Bedford, Mass. and quantitated with QuantArray™ software also from GSI Lumonics. Analysis of antibodies bound to microarrays detects a lower limit of 0.5 picograms of human IgG or IgM bound to a microarray (See Mezzasoma et al., ibid.).

After analysis is complete, the principal game unit transmits data to a network. The data may include quantitative data on anti-viral antibodies including IgA, IgG, and IgM antibodies for multiple viruses that are detected in salivary fluid from the individual playing the game. For example, the amounts of IgG antibodies specific for CMV, HSV, rubella virus, measles virus, mumps virus, respiratory syncytial virus, Epstein-Barr virus, hepatitis A virus, hepatitis B virus, hepatitis C virus and/or HIV present in the salivary fluid of an individual may be transmitted to a computer network that may be accessed by the individual game player, family members, teachers, healthcare workers, public health officials and caregivers. Moreover, information stored on the network may include the individual's medical history, vaccination schedule, previous viral infections, medications and known allergic responses. Importantly the network contains data from previous analyses by the game system of anti-viral antibody levels that constitute baseline, or pre-infection, or pre-vaccination levels of antibodies for specific microbial pathogens.

As an illustration, FIG. 14 depicts a game system 100 including a game component 113 placed into an indentation 1435 in a principal game unit 120. The principal game unit 120 includes microfluidic devices 1440 operably connected to the indentation 1435 and configured to wash the viral antigen microarray 1425 as described above. The principal game unit 120 also includes a scanning confocal microscope 1445 configured to detect antibodies bound to the microarray 1425 after washing is completed. The principal game unit also includes circuitry 1465 and a power source 1460. A light 1450 is operably connected to the scanning confocal microscope 1445 and circuitry 1465 and configured to indicate when the analysis is complete so that the game component 113 may be removed. The principal game unit 120 also includes an antenna 1455 operably connected to the circuitry and configured to send a signal 150 with information regarding the analysis to a computing device 160. The principal game unit 120 further includes an antenna 1455 operably connected to the circuitry and configured to receive a signal 155 from a computing device 160. For example, a computing device 160 may send a signal 155 confirming that a signal 150 from the principal game unit 120 has been received. Results are indicated to a system user 165, such as on a user interface display of a computing device 160.

### Example 2

### Game System to Detect Infectious Bacterial Pathogens in Saliva and Indicate their Presence to Individual Users, Healthcare Workers and Caregivers

A game system is described to detect bacterial pathogens in saliva and/or nasal fluid from individuals playing the game and to indicate their presence to individual users, caregivers and healthcare workers. Game systems comprised of game components and a principal game unit are configured to be used by children, students, hospital patients, institutional inmates and the elderly. The game components are configured to sample salivary fluid, which often includes nasal fluid, and detect pathogenic bacteria using a sensor system including an aptamer array. The aptamer array electronically signals a principal game unit that is configured to analyze the electronic signals and transmit information on bacterial pathogens to a network comprised of the individual game player, caregivers, teachers, parents and healthcare workers.

Game components may have a semi-permeable membrane covering an opening to the interior of the game component that allows salivary fluid and its contents to pass into the interior but excludes macroscopic particles, food and other constituents that might interfere with analysis of the salivary fluid and its contents. For example, devices to collect salivary fluid are described in U.S. Pat. No. 6,022,326 to Tatum et al., entitled “Device and method for automatic collection of whole saliva,” which is incorporated herein by reference. A semi-permeable membrane covering may include biocompatible membranes such as those described in U.S. Pat. No. 6,258,870 to Hubbell et al., titled “Gels for encapsulation of biological materials,” which is herein incorporated by reference.

The game component contains a biosensor for pathogenic bacteria that is based on an aptamer recognition element. A biosensor with aptamer recognition elements that bind biotoxins or bacteria and contain electrochemical elements transduces an electrical signal (see Lai et al., “Aptamer-based electrochemical detection of picomolar platelet-derived growth factor directly in blood serum,” Anal. Chem. 79: 229-233 (2007), which is incorporated herein by reference) and indicates the presence of pathogenic bacteria in salivary fluid and nasal fluid.

More specifically, aptamers specific for E. coli 0111:B4 are selected from a random oligonucleotide collection by using magnetic beads conjugated with lipopolysaccharide (LPS) 0111:B4 (see Dwarakanath et al., “Quantum dot-antibody and aptamer conjugates shift fluorescence upon binding bacteria,” BBRC 325: 739-743 (2004) and Bruno and Kiel, “Use of magnetic beads in selection and detection of biotoxin aptamers by electrochemiluminescence and enzymatic methods,” BioTechniques, 32: 178-183 (2002) which are herein incorporated by reference). The biotoxin LPS 0111:B4 (available from Sigma-Aldrich, St. Louis, Mo.) is conjugated to Dynal M-270 amine-magnetic beads (available from Invitrogen Corp., Carlsbad, Calif.) using sodium periodate and cyanoborohydride chemistry as described by Dwarakanath et al., ibid. Methods for construction, selection and amplification of a single stranded, random sequence DNA pool containing approximately 2×10¹⁴ different molecules are described in U.S. Pat. No. 5,631,146 to Szostak et al., titled “DNA aptamers and catalysts that bind adenosine or adenosine-5′-phosphates and methods for isolation thereof,” which is herein incorporated by reference. To select aptamers that recognize E. coli 0111:B4, the random oligonucleotide sequence pool is incubated with and allowed to bind to LPS 0111:B4-magnetic beads. The aptamer-LPS-magnetic bead complexes are washed three times and then polymerase chain reaction (“PCR”) is used to amplify the aptamers bound to the LPS-magnetic beads. PCR reagents, protocols and thermal cyclers are available from Applied Biosystems, Foster City, Calif. Selected and amplified oligonucleotides are added to another aliquot of LPS-magnetic beads and the entire process is repeated four more times (see Bruno et al., ibid.).

Binding of selected aptamers to E. coli 0111:B4 is verified by attachment of quantum dots to the aptamers and performance of fluorescence spectroscopy. Quantum dots (also known as nanocrystals) are available from eBioscience, Inc. (San Diego, Calif.). Protocols for attaching quantum dots to oligonucleotides via a N-b-maleimidopropionic acid (“BMPA”; Thermo Fisher Scientific Inc., Rockford, Ill.) linkage are detailed in Dwarakanath et al., ibid. Selected aptamers with quantum dots attached specifically bind to E. coli 0111:B4 (available from American Type Culture Collection, Manassus, Va.), inducing a shift in the fluorescence emission wavelength of the quantum dots (as shown by Dwarakanath et al., ibid.). The maximum emission wavelengths (lambdaₘₐₓ) for quantum-dot aptamers alone (lambdaₘₐₓ=˜605 nm) and quantum dot aptamers plus E. coli (lambdaₘₐₓ=˜462 nm) are visible with less than approximately 2.8×10⁶ bacteria per mL. Fluorescence emission spectra can be determined with a spectrofluorometer (available from StellarNet Inc., Tampa, Fla.).

Selected aptamers that specifically bind E. coli (or other microbes, as indicated for the embodiment) are used to initiate the action of electronic signaling elements in a game component. The binding of specific E. coli proteins to the aptamer results in the creation of a signal, which electronic elements of the principal game unit detect as the presence of microbial contaminants. Aptamers that provide for the electronically detectable signals are created by mutagenesis of aptamers followed by conjugation of an oxidation/reduction tag to the mutated aptamer. See Stojanovic et al., “Aptamer-based folding fluorescent sensor for cocaine,”J. Am. Chem. Soc., 123: 4928-4931 (2001) and Baker et al., “An electronic, aptamer-based small-molecule sensor for the rapid, label-free detection of cocaine in adulterated samples and biological fluids,” J. Am. Chem. Soc., 128: 3138-3139 (2006), which are herein incorporated by reference. Aptamers which signal electronically upon binding a specific target protein are also described by Lai et al., “Aptamer-based electrochemical detection of picomolar platelet-derived growth factor directly in blood serum,” Anal. Chem. 79: 229-233 (2007), which is herein incorporated by reference. Covalent attachment of the electroactive label methylene blue (“MB”) to the 3′ end of a DNA aptamer specific for a target protein creates an electroactive aptamer that signals via electron transfer when the target (e.g. protein) binds. Methods for covalent attachment of MB to an aptamer using an N-hydroxysuccinimide ester of MB to create a MB-aptamer are described by Lai et al., ibid.

A game component with an aptamer-modified electrode is fabricated to include a battery, a direct current/alternating current transformer, a reference electrode (e.g. Ag/AgCl electrode), microcircuitry and signaling elements. A specific target protein sensor is constructed by immobilization of the MB-aptamer on gold electrodes. Gold working electrodes (0.88 mm²) are fabricated on a glass plate using standard microfabrication techniques. See Lai et al., “Differential labeling of closely spaced biosensor electrodes via electrochemical lithography,” Langmuir, 22: 1932-1936 (2006), which is incorporated by reference herein. Aptamer electrodes can be analyzed in a principal game unit by alternating current voltammetry over the range −0.15 to −0.43 Volt versus Ag/AgCl with a 10 Hz, 25-mV ac potential (as shown by Lai et al., (2007), ibid.). A platinum wire is used as the counter electrode and electrochemical potentials are reported versus a Ag/AgCl (3 M KCl) reference electrode. Methods and materials including voltammetry instrumentation and experimental parameters are detailed in Lai et al., (2007), ibid. Aptamer-MB biosensors are extremely sensitive. For example, the measured dynamic range for a platelet-derived growth factor (PDGF) sensor is 50 pM to 10 nM PDGF as shown by Lai et al. (2007), ibid. Aptamer-MB biosensors are capable of detecting microbes or microbial toxins in salivary fluid at very low concentrations.

Game systems with bacterial biosensors and principal game units are configured to transmit signals electronically to external devices such as cell phones, computing devices, personal digital assistants (PDAs) or other devices, which may be part of a network. Detection of bacterial pathogens in an individual's salivary fluid may be automatically indicated to the individual and to a system user via the network to alert caregivers, parents, teachers, healthcare workers and public health officials of an apparent infection.

### Example 3

### A Game System to Detect Medicinal Analytes in Salivary Fluid from an Individual and to Indicate the Concentrations of Medicinal Analytes to the Individual, to Caregivers and to Healthcare Workers

A game system is configured to detect medicinal analytes in salivary fluid from patients playing a game and to indicate the concentrations of medicinal analytes to individual game players, family members, caregivers and healthcare workers. Such a game system may be used to confirm that patients prescribed ongoing courses of medication are compliant in taking the appropriate medications at prescribed dosages and that the medications are being metabolized by the individuals' bodies as expected. Compliance with prescribed treatment regimens is recognized as a substantial problem in ongoing treatment regimens, particularly in chronic conditions such as psychiatric disease. Often it is difficult for a healthcare professional to determine if the prescribed therapy is not successful due to lack of compliance with the drug regimen or to lack of therapeutic effect. See Cramer and Rosenheck, “Compliance with medication regimens for mental and physical disorders,” Psychiatr Sery 49:196-201 (1998) and Mitchell and Selmes, “Why don't patients take their medicine? Reasons and solutions in psychiatry,” Advances in Psychiatric Treatment 13: 336-346 (2007), which are herein incorporated by reference.

A game system is configured to detect the concentration of medicinal analytes in salivary fluid and signal a system user that medication levels are within or outside preset concentration limits. Preset concentration limits may be established in blood, and the game system configured to convert the concentration levels based on the correlation between medication concentrations in salivary fluid and blood (serum or plasma). During game play activity the game components, which incorporate hydrogels configured for colorimetric change in the presence of medicinal analytes, are placed in a principal game unit and analyzed by a sensor system that is configured to detect colorimetric changes. The game system includes a transmitter that is configured to transmit information regarding the detection to an external device, for example a computer or cell phone, to indicate the medication concentrations to a caregiver. Information regarding the detection may be saved in memory in the external device for comparison with similar detection information taken at other times. The external device may be part of a network which may be accessed by the individual game player, parents, caregivers and healthcare workers. A game system detecting and reporting frequently on medicinal analyte levels is useful for patients who need to monitor their medication levels, and to caregivers who need to monitor patients and verify compliance with prescribed treatments.

A game system including a sensor system configured to detect medications in salivary fluid can be used to detect psychiatric medications and to monitor patient compliance. For example, a game system can detect lithium, carbamazepine, ethosuximide, phenobarbital, phenyloin, and theophylline in salivary fluid and indicate if preset criteria for drug concentration (i.e. therapeutic concentrations) are being maintained. Serum concentrations and salivary fluid concentrations have been shown to be highly correlated for: lithium (correlation coefficient, r=0.87), carbamazepine (r=0.89), phenobarbital (r=0.98), phenyloin (r=0.97), and theophylline (r=0.85). See Kaufman and Lamster, “The diagnostic applications of saliva—a review,” Crit. Rev. Oral Biol. Med., 13: 197-212 (2002), which is herein incorporated by reference. Phenyloin is a commonly prescribed anti-seizure medication. The concentration of phenyloin present in salivary fluid has been shown to correlate with therapeutically active phenyloin levels, even in the presence of the commonly co-prescribed medication sodium valproate. See Knott et al., ibid., which is herein incorporated by reference.

A game system with a game component including a hydrogel sensor that detects phenyloin and indicates its presence via a color change may be used periodically (e.g. approximately every hour, every 4 hours, every 8 hours, every 12 hours, daily, weekly) to assess the level of phenyloin in salivary fluid. A hydrogel sensor that changes color in response to phenyloin is fabricated to include crystalline colloid arrays including charged polystyrene spheres that are polymerized within the hydrogel. The hydrogel is configured to swell or shrink in response to phenyloin present in salivary fluid. See Holtz and Asher, “Polymerized colloidal crystal hydrogel films as intelligent chemical sensing materials,” Nature 389: 829-832 (1997), which is incorporated herein by reference). Methods and materials to produce hydrogels with covalently coupled antibodies and antigens that swell in response to cognate antigen are described in Miyata et al., “A reversibly antigen-responsive hydrogel,” Nature 399: 766-769 (1999), which is incorporated herein by reference. Antibodies that specifically recognize psychiatric medications, for example, carbamazepine, phenobarbital, phenyloin, or theophylline, are available from GenWay Biotech, Inc. (San Diego, Calif.). Crystalline colloidal arrays can diffract light at (visible) wavelengths determined by their lattice spacing, which can give rise to an intense color. Swelling of the hydrogel comprising the polymerized crystalline colloid array changes the lattice spacing and causes a shift in the Bragg peak of diffracted light to longer wavelengths. For example, a polymerized crystalline colloid that swells in response to lead acetate (Pb(CH3COO)₂) shifts its Bragg peak diffraction wavelength from 450 nm to approximately 500 nm, a shift easily visible by the naked eye (Holtz and Asher et al., ibid.).

A game system with a game component is fabricated to contain a polymerized crystalline colloid with a phenyloin-anti-phenyloin antibody complex conjugated to the hydrogel. A game system including such a game component may be used by an individual game player to capture and recognize phenyloin in their salivary fluid. Enclosure of the game component with a semi-permeable membrane allows salivary fluid and phenyloin, but not interfering substances, to enter the game component and to contact the antibodies on the hydrogel leading to swelling of the hydrogel and a shift in the diffraction wavelength corresponding to a visible color change. For example, Holtz and Asher, ibid., report polymerized crystalline colloid arrays that can respond within approximately 30 seconds to 2 minutes with a change in color from blue to green. In addition, the game component can be placed in a principal game unit including a spectrophotometer to measure the peak diffraction wavelength and the shift in peak diffraction wavelength, which can be correlated with analyte concentration. (See Holtz and Asher et al., ibid.)

Moreover, the game component containing a polymerized crystalline colloidal array may be reused following a wash in an analyte-free solution. For example, as shown by Holtz and Asher et al., ibid., a previously used polymerized crystalline colloid array sensor diffracts light at its original peak wavelength after soaking in vitro in deionized water, and it is fully responsive upon reimmersion in an analyte solution. A game component containing a polymerized crystalline colloid array medication sensor may be used to periodically (e.g. approximately every 4 hours, 8 hours, 16 hours, 24 hours, 48 hours or 72 hours) assess and indicate medication levels (e.g. phenyloin) in salivary fluid. A color change may be visually detected by an individual present during game play, and reported via an external device, such as a cell phone or computer, and received by a network system that stores the color, date, time and identity of the patient. Alternatively, the game component may be analyzed in a principal game unit including a detection device that measures the peak wavelength of diffraction and sends the wavelength data, date, time, and identity of the patient to a network system. Devices to measure peak wavelength of diffraction include spectrophotometers and colorimeters (available from Hach Co, Loveland, Colo. and Cole-Parmer Instrument Co., Vernon Hills, Ill.) and are operably connected to a computer device configured to receive wavelength data, to calculate analyte concentrations, and to send the information to a network system.

### Example 4

### A Game System to Detect Recreational Drugs in Salivary Fluid from an Individual Player and Indicate the Levels of Recreational Drugs to the Individual, and to Caregivers and Healthcare Workers

A game system is described to detect recreational drugs (e.g. methamphetamine, tetrahydrocannabinol (THC), ethanol, cocaine) in salivary fluid from individuals playing the game and to indicate the concentrations of recreational drugs to individual users, parents, caregivers and healthcare workers. A sensor system that includes game components (e.g. modules, remotes, wands, figures, icons) collects salivary fluid, detects drugs, and transmits signals regarding the detection. Signals are detected by a principal game unit and information on recreational drug presence is transmitted to a network that may include the individual game player, his or her family members, teachers, caregivers and healthcare workers. The game system can be used by families, individuals, schools, drug rehab centers, video game parlors, and any other institution or business where routine recreational drug monitoring may be desirable.

A game system is described to detect cocaine in salivary fluid from cocaine users who play the game system, and simultaneously monitor their cocaine usage. The game system includes a sensor system to detect cocaine that is based on an aptamer that signals electronically when cocaine is present. To create the system, an aptamer that specifically binds cocaine with high affinity and high specificity is selected from a mixture (or pool) of oligonucleotides with random sequences by using an iterative process combining affinity chromatography and amplification termed Systematic Evolution of Ligands by Exponential Enrichment (SELEX; see U.S. Pat. No. 5,475,096 issued to Gold et al., titled, “Nucleic acid ligands,” which is herein incorporated by reference). Construction, selection and amplification of a single stranded, random sequence DNA pool containing approximately 2×10¹⁴ different molecules is described in U.S. Pat. No. 5,631,146 issued to Szostak et al. titled, “DNA aptamers and catalysts that bind adenosine or adenosine-5′-phosphates and methods for isolation thereof,” which is herein incorporated by reference. DNA ligands are selected by affinity chromatography with a matrix comprised of sepharose or agarose coupled to cocaine. Methods and materials for construction and use of an affinity chromatography matrix are given in Win et al., “Codeine-binding RNA aptamers and rapid determination of their binding constants using a direct coupling surface plasmon resonance assay,” Nucleic Acids Research, 34: 5670-5682 (2006) which is herein incorporated by reference. Following each round of selection, amplification is carried out using the polymerase chain reaction as described (U.S. Pat. No. 5,631,146, Szostak et al., ibid. and Win et al., ibid.). Alternatively an aptamer with high affinity for cocaine may be purchased from AptaRes, (D-15749 Mittenwalde, Germany).

Aptamers that signal electronically are created by mutagenesis of aptamers and by conjugation of an oxidation/reduction tag to the aptamer. See Stojanovic et al., “Aptamer-based folding fluorescent sensor for cocaine,”J. Am. Chem. Soc., 123: 4928-4931 (2001) and Baker et al., “An electronic, aptamer-based small-molecule sensor for the rapid, label-free detection of cocaine in adulterated samples and biological fluids,” J. Am. Chem. Soc., 128: 3138-3139 (2006), which are herein incorporated by reference. An aptamer configured to bind cocaine and result in a comformational change is created by a combination of site-specific and random mutagenesis as shown by Stojanovic et al., ibid. Conjugation of an oxidation/reduction taggant, such as methylene blue, to an aptamer that undergoes a ligand-dependent conformational change will result in an aptamer that signals electrochemically when ligand binds. For example, conjugation of methylene blue (available from ScienceLab.com, Inc., Houston, Tex.) to an aptamer that changes conformation upon binding cocaine creates an electrochemical aptamer-based sensor that will transfer electrons in response to cocaine binding (see Baker, et al., ibid.). A cocaine-specific aptamer conjugated with methylene blue is immobilized on a 1.6 mm diameter gold electrode (Bioanalytical Systems, Inc., West Lafayette, Ind.) by adding an alkanethiol group to the 5′ end of the aptamer and reacting the derivatized aptamer-thiol with the gold surface (see Baker et al., ibid. for methods and materials).

To detect the electrochemical response of the electronic aptamer cocaine sensor, one can perform alternating current voltammetry with an electrochemical workstation (CH Instruments, Austin, Tex.) using a reference electrode comprised of Ag and AgCl. An increase in current following immersion of an electronic aptamer cocaine sensor in cocaine solutions is observed and displayed as voltammograms plotting volts versus alternating current. An electronic aptamer-based sensor for cocaine is sensitive to less than 10 uM cocaine and exhibits a response curve that is informative to approximately 500 uM cocaine (as shown by Baker et al., ibid.). Additional examples of aptamer-based electrochemical sensors, including those configured to respond to small molecules (e.g. nicotine), proteins (including thrombin and platelet-derived growth factor) and cells are as described in Lee et al., “Aptamers as molecular recognition elements for electrical nanobiosensors,” Anal. Bioanal. Chem., 390: 1023-1032 (2008) which is herein incorporated by reference.

A game system with an electronic aptamer-based cocaine sensor integrated in a game component also includes a principal game unit that contains a micro-voltammetric sensor that can measure changes in current when varying voltages are applied. The principal game unit includes the micro-voltammetric sensor in an indentation configured to mate with the individual game component during game play (e.g. as depicted in FIG. 6). Microfabricated electrochemical sensors are described in Liu et al., “Applications of microfabrication techniques in electrochemical sensor development,” Applied Biochemistry and Biotechnology, 41: 99-107 (1993), which is herein incorporated by reference. Salivary fluid, which may contain cocaine, enters a chamber in the game component via capillary action and comes in contact with the electronic aptamer-based sensor for cocaine. The electronic signal measured by voltammetry (as above) is detected by circuitry in the principal game unit. The electronic signal detected by voltammetry is transmitted by the principal game unit to an external device such as a computer, a cell phone or an ammeter. Based on the transmitted information, a computer device may calculate the concentration of cocaine in salivary fluid and, by correlation, in blood serum based on previously established curves (see Kaufman and Lamster, “The diagnostic applications of saliva—a review,” Crit. Rev. Oral Biol. Med., 13: 197-212 (2002) which is herein incorporated by reference). Circuitry in the game component and the principal game unit may identify the individual game player and transmit electronic signals including information on the time, date, and location, along with the salivary fluid cocaine concentration. Such information may be indicated with computers, cell phones and other devices possessed by the individual game player, parents, teachers, caregivers, healthcare workers, probation officers and other authorized individuals.

A game system including one or more modular drug sensor may be used to monitor drug abuse as well as recreational drugs. For example, amphetamines, barbiturates, opioids, cocaine, tetrahydrocannabinol, and nicotine can be detected in salivary fluid (Kaufman and Lamster, ibid.), and the detected drug concentrations in saliva or the calculated drug concentrations in serum reported to caregivers or authorities. Although details are given herein regarding the construction of a game system for monitoring cocaine in an individual user's salivary fluid, similar methodologies may be implemented to fabricate game systems configured to monitor other medications or drugs with a high correlation between serum concentrations and salivary fluid concentrations (e.g. see Kaufman and Lamster, ibid.).

### Example 5

### A Game System to Detect Biomarker Chemicals in the Breath of Individuals Who May have Asthma or Lung Inflammatory Diseases and to Indicate Biomarker Concentrations to a Network of Caregivers

A game system is described that is configured to sample and assay the exhaled breath of individual game players. The game system is used to screen individuals (in particular children, adolescents and young adults) for asthma or lung inflammatory disease, and/or to monitor individuals with previously diagnosed asthma or lung inflammatory disease in order to aid in controlling asthma and lung inflammation together with ongoing treatments. The game system includes game components (e.g. figures, remotes, wands, icons, joysticks) configured to sample exhaled breath from the individual player and detect chemicals, metabolites, and biological molecules. Furthermore, each game component contains a unique radio frequency identification (RFID) tag that is assigned to each individual game player. The game system also includes a principal game unit that mates with the game components and determines the identity and level of analytes present in the exhaled breath captured by each game component. The principal game unit also includes a RFID reader that identifies a specific game component, and therefore the associated game player, by the associated RFID tag. The principal game unit is configured to transmit the detected breath analyte information to external devices (e.g. cell phones and computer devices). The information may be transmitted to a network and results indicated to the individual game player, family members, teachers, healthcare workers and caregivers. An external device also stores the individual's medical information that corresponds to the unique RFID tag including: medical history, previous analyses of exhaled breath, and past and present medications.

Individual game components in the game system include unique RFID tags. RFID tags are assigned to each individual player by scanning the game component containing an RFID tag over a principal game unit containing an RFID reader module. The game system is configured to not allow game play activity of a game component that has not been scanned. Game components, uniquely identified by RFID tags, may be discarded after playing the game and the RFID tag may be transferred to a fresh game component with each player retaining their unique RFID tag. Alternatively, a reusable game component may include a RFID reader module and each individual may be assigned a unique RFID tag incorporated in a bracelet or neck tag. RFID tags and reader modules suitable for healthcare, event management, access control and asset tracking are available from GAO RFID Inc., Seattle, Wash.

The game system including the game components and the principal game unit contains a sensor system to detect and quantitate the level of breath analytes that are biomarkers for lung inflammation and asthma. Game play activity is coordinated to include individual players exhaling into the breathing tube of their individual game component. For example a child with previously diagnosed asthma breathes into a game component and exhaled nitric oxide is detected and quantitated by the sensor system. Each game component includes a breathing tube connected to a sensor within the game component. Each game component contains an electrochemical sensor for detection and quantitation of nitric oxide in exhaled breath. See Hemmingsson et al., “Novel hand-held device for exhaled nitric oxide-analysis in research and clinical applications,” J. Clin. Monitoring and Computing 18: 379-387 (2004), which is incorporated herein by reference. Handheld nitric oxide analyzers are available from Aerocrine AB (Solna, Sweden) that measure fractional nitric oxide levels in exhaled breath in concentrations ranging from 5 to 300 parts per billion of exhaled breath. Each game component is configured to mate with a principal game unit and transmit the individual's fractional exhaled nitric oxide (FENO) level to the principal game unit where the data is stored and analyzed.

Analysis of FENO for asthmatic children may be used as a biomarker for asthma control and may guide treatment with corticosteroids as shown by Robroeks et al., “Exhaled nitric oxide and biomarkers in exhaled breath condensate indicate the presence, severity and control of childhood asthma,” Clin. Exp. Allergy 37: 1303-1311 (2007) and Smith et al., “Use of exhaled nitric oxide measurements to guide treatment in chronic asthma,” N. Engl. J. Med. 352: 2163-73 (2005) which are incorporated herein by reference. The mean daily dose of an inhaled corticosteroid, fluticasone, may be reduced from 641 micrograms per day to 270 micrograms per day by monitoring exhaled nitric oxide levels and reducing the dosage stepwise as clinically appropriate. A criterion to maintain FENO at or below 15 parts per billion allows reducing corticosteroid usage, while the rate of asthma exacerbations (e.g. 0.49 episodes per patient per year) is equivalent to that seen with higher doses of corticosteroids. See Smith et al., ibid. Thus a game system with a sensor system for nitric oxide monitoring of exhaled breath allows the reduction of maintenance dosing of inhaled corticosteroids for chronic asthma patients without compromising asthma control.

FENO levels are transmitted from game components to the principal game unit and assigned to the individual file corresponding to a unique RFID tag. Results are indicated directly with a display integrated into the principal game unit. For example, depending on the game play activity and then age range of the players involved, a red light may flash indicating that one or more game players requires prompt medical intervention. The principal game unit also transmits the FENO level and patient identification to a computing device (e.g. computer, cell phone, PDA) in which previous FENO analyses, past and present medications and medical history are stored. Ultimately FENO levels and treatment recommendations are indicated to system users such as the individual patient, caregivers, parents and healthcare workers.

The state of the art has progressed to the point where there is little distinction left between hardware, software, and/or firmware implementations of aspects of systems; the use of hardware, software, and/or firmware is generally (but not always, in that in certain contexts the choice between hardware and software can become significant) a design choice representing cost vs. efficiency tradeoffs. There are various vehicles by which processes and/or systems and/or other technologies described herein can be effected (e.g., hardware, software, and/or firmware), and the preferred vehicle will vary with the context in which the processes and/or systems and/or other technologies are deployed. For example, if an implementer determines that speed and accuracy are paramount, the implementer may opt for a mainly hardware and/or firmware vehicle; alternatively, if flexibility is paramount, the implementer may opt for a mainly software implementation; or, yet again alternatively, the implementer may opt for some combination of hardware, software, and/or firmware. Hence, there are several possible vehicles by which the processes and/or devices and/or other technologies described herein may be effected, none of which is inherently superior to the other in that any vehicle to be utilized is a choice dependent upon the context in which the vehicle will be deployed and the specific concerns (e.g., speed, flexibility, or predictability) of the implementer, any of which may vary. For example, the optical aspects of implementations will typically employ optically-oriented hardware, software, and or firmware.

In some implementations described herein, logic and similar implementations may include software or other control structures. Electronic circuitry, for example, may have one or more paths of electrical current constructed and arranged to implement various functions as described herein. In some implementations, one or more media may be configured to bear a device-detectable implementation when such media hold or transmit a device detectable instructions operable to perform as described herein. In some variants, for example, implementations may include an update or modification of existing software or firmware, or of gate arrays or programmable hardware, such as by performing a reception of or a transmission of one or more instructions in relation to one or more operations described herein. Alternatively or additionally, in some variants, an implementation may include special-purpose hardware, software, firmware components, and/or general-purpose components executing or otherwise invoking special-purpose components. Specifications or other implementations may be transmitted by one or more instances of tangible transmission media as described herein, optionally by packet transmission or otherwise by passing through distributed media at various times.

Alternatively or additionally, implementations may include executing a special-purpose instruction sequence or invoking circuitry for enabling, triggering, coordinating, requesting, or otherwise causing one or more occurrences of virtually any functional operations described herein. In some variants, operational or other logical descriptions herein may be expressed as source code and compiled or otherwise invoked as an executable instruction sequence. In some contexts, for example, implementations may be provided, in whole or in part, by source code, such as C++, or other code sequences. In other implementations, source or other code implementation, using commercially available and/or techniques in the art, may be compiled, implemented, translated, or converted into a high-level descriptor language (e.g., initially implementing described technologies in C or C++ programming language and thereafter converting the programming language implementation into a logic-synthesizable language implementation, a hardware description language implementation, a hardware design simulation implementation, and/or other such similar mode(s) of expression). For example, some or all of a logical expression (e.g., computer programming language implementation) may be manifested as a Verilog-type hardware description (e.g., via Hardware Description Language (HDL) and/or Very High Speed Integrated Circuit Hardware Descriptor Language (VHDL)) or other circuitry model which may then be used to create a physical implementation having hardware (e.g., an Application Specific Integrated Circuit). Those skilled in the art will recognize how to obtain, configure, and optimize suitable transmission or computational elements, material supplies, actuators, or other structures in light of these teachings.

In a general sense, the various aspects described herein can be implemented, individually and/or collectively, by a wide range of hardware, software, firmware, and/or any combination thereof and can be viewed as being composed in part of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of memory (e.g., random access, flash, read only, etc.)), and/or electrical circuitry forming a communications device (e.g., a modem, communications switch, optical-electrical equipment, etc.). The subject matter described herein may be implemented in an analog or digital fashion or some combination thereof.

At least a portion of the devices and/or processes described herein can be integrated into a data processing system. A data processing system generally includes one or more of a system unit housing, a video display device, memory such as volatile or non-volatile memory, processors such as microprocessors or digital signal processors, computational entities such as operating systems, drivers, graphical user interfaces, and applications programs, one or more interaction devices (e.g., a touch pad, a touch screen, an antenna, etc.), and/or control systems including feedback loops and control motors (e.g., feedback for sensing position and/or velocity; control motors for moving and/or adjusting components and/or quantities). A data processing system may be implemented utilizing suitable commercially available components, such as those typically found in data computing/communication and/or network computing/communication systems.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In one embodiment, several portions of the subject matter described herein may be implemented via Application Specific Integrated Circuits (ASICs), Field Programmable Gate Arrays (FPGAs), digital signal processors (DSPs), or other integrated formats. However, some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a floppy disk, a hard disk drive, a Compact Disc (CD), a Digital Video Disk (DVD), a digital tape, a computer memory, etc.; and a transmission type medium such as a digital and/or an analog communication medium (e.g., a fiber optic cable, a waveguide, a wired communications link, a wireless communication link (e.g., transmitter, receiver, transmission logic, reception logic, etc.), etc.).

The herein described components (e.g., operations), devices, objects, and the discussion accompanying them are used as examples for the sake of conceptual clarity and various configuration modifications are contemplated. Consequently, as used herein, the specific exemplars set forth and the accompanying discussion are intended to be representative of their more general classes. In general, use of any specific exemplar is intended to be representative of its class, and the non-inclusion of specific components (e.g., operations), devices, and objects should not be taken limiting. The foregoing specific exemplary processes and/or devices and/or technologies are representative of more general processes and/or devices and/or technologies taught elsewhere herein, such as in the claims filed herewith and/or elsewhere in the present application.

The herein described subject matter sometimes illustrates different components contained within, or connected with, different other components. It is to be understood that such depicted architectures are merely exemplary, and that in fact many other architectures may be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled,” to each other to achieve the desired functionality, and any two components capable of being so associated can also be viewed as being “operably couplable,” to each other to achieve the desired functionality. Specific examples of operably couplable include but are not limited to physically mateable and/or physically interacting components, and/or wirelessly interactable, and/or wirelessly interacting components, and/or logically interacting, and/or logically interactable components.

It is common within the art to implement devices and/or processes and/or systems, and thereafter use engineering and/or other practices to integrate such implemented devices and/or processes and/or systems into more comprehensive devices and/or processes and/or systems. That is, at least a portion of the devices and/or processes and/or systems described herein can be integrated into other devices and/or processes and/or systems via a reasonable amount of experimentation. Examples of such other devices and/or processes and/or systems might include—as appropriate to context and application—all or part of devices and/or processes and/or systems of (a) an air conveyance (e.g., an airplane, rocket, helicopter, etc.), (b) a ground conveyance (e.g., a car, truck, locomotive, tank, armored personnel carrier, etc.), (c) a building (e.g., a home, warehouse, office, etc.), (d) an appliance (e.g., a refrigerator, a washing machine, a dryer, etc.), (e) a communications system (e.g., a networked system, a telephone system, a Voice over IP system, etc.), (f) a business entity (e.g., an Internet Service Provider (ISP) entity such as Comcast Cable, Qwest, Southwestern Bell, etc.), or (g) a wired/wireless services entity (e.g., Sprint, Cingular, Nextel, etc.), etc.

In certain cases, use of a system or method may occur in a territory even if components are located outside the territory. For example, in a distributed computing context, use of a distributed computing system may occur in a territory even though parts of the system may be located outside of the territory (e.g., relay, server, processor, signal-bearing medium, transmitting computer, receiving computer, etc. located outside the territory). A sale of a system or method may likewise occur in a territory even if components of the system or method are located and/or used outside the territory. Further, implementation of at least part of a system for performing a method in one territory does not preclude use of the system in another territory.

All of the above U.S. patents, U.S. patent application publications, U.S. patent applications, foreign patents, foreign patent applications and non-patent publications referred to in this specification and/or listed in any Application Data Sheet, are incorporated herein by reference, to the extent not inconsistent herewith.

While particular aspects of the present subject matter described herein have been shown and described, it will be apparent that, based upon the teachings herein, changes and modifications may be made without departing from the subject matter described herein and its broader aspects and, therefore, the appended claims are to encompass within their scope all such changes and modifications as are within the true spirit and scope of the subject matter described herein. In general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). If a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense of the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense of the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

With respect to the appended claims, recited operations therein may generally be performed in any order. Also, although various operational flows are presented in sequence(s), it should be understood that the various operations may be performed in other orders than those which are illustrated, or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

The various aspects and embodiments disclosed herein are for purposes of illustration and are not intended to be limiting, with the true scope and spirit being indicated by the following claims.

What is claimed is:

1. A game system, comprising:
at least one game component configured for use in a game system by an individual player;
at least one principal game unit including a port configured for communication with the at least one game component;
at least one sensor, wherein the at least one sensor is configured to detect one or more analyte obtained from the individual player; and
at least one signal output unit associated with the at least one sensor and configured to output signal in response to at least one sensed analyte.

2. The game system of claim 1, wherein the at least one game component is configured to encircle part of the individual player's body.

3. The game system of claim 1, wherein the at least one game component comprises:
at least one flavorant.

4.-7. (canceled)

8. The game system of claim 1, wherein the at least one game component comprises:
a transceiver system configured to receive signals from the at least one principal game unit.

9. The game system of claim 1, wherein the at least one game component comprises:
at least one unique identifier.

10. The game system of claim 1, wherein the at least one game component comprises:
at least one transmitter.

11. The game system of claim 1, wherein the at least one game component comprises:
at least one selective medium.

12. The game system of claim 1, wherein the at least one game component comprises:
at least one reservoir.

13.-18. (canceled)

19. The at least one game component of claim 1, comprising:
an encapsulating material.

20.-24. (canceled)

25. The game system of claim 1, wherein the at least one sensor comprises:
at least one recognition element.

26.-27. (canceled)

28. The game system of claim 1, wherein the one or more analyte comprises:
at least one moiety that is an indicator of a physiological state.

29. The game system of claim 1, wherein the one or more analyte comprises:
a metabolite.

30.-31. (canceled)

32. The game system of claim 1, wherein the at least one sensor is configured to be indirectly responsive to the one or more analyte.

33.-35. (canceled)

36. The game system of claim 1, wherein the at least one principal game unit comprises:
at least one detector configured to detect a signal from the at least one sensor.

37.-38. (canceled)

39. The game system of claim 1, wherein the at least one principal game unit comprises:
at least one transceiver system configured to receive signals from the at least one game component.

40. The game system of claim 1, wherein the at least one principal game unit comprises:
at least one transceiver system configured to receive signals from at least one external network system.

41. The game system of claim 1, wherein the at least one principal game unit comprises:
at least one signal transmitter.

42. The game system of claim 1, wherein the at least one principal game unit comprises:
at least one unique identifier.

43. The game system of claim 1, wherein the at least one principal game unit comprises:
at least one region configured to pair with the at least one game component.

44. The game system of claim 1, further comprising:
at least one physiological sensor.

45. (canceled)

46. The game system of claim 1, comprising:
at least one signal emitter.

47.-52. (canceled)

53. The game system of claim 1, comprising:
one or more display unit configured to be responsive to the at least one sensor.

54. The game system of claim 1, comprising:
one or more display unit configured to be responsive to the at least one principal game unit.

55.-56. (canceled)

57. The game system of claim 1, comprising:
at least one antenna operably connected to the at least one principal game unit.

58. The game system of claim 1, comprising:
at least one antenna operably connected to the at least one game component.

59. The game system of claim 1, comprising:
at least one user interface device operably connected to the at least one principal game unit.

60. A game system comprising:
at least one game component configured for use in a game system by an individual player;
at least one sensor system operably connected to the at least one game component and configured to detect one or more analyte, the at least one sensor system including a signal transmitter;
at least one signal detector configured to detect a signal transmitted from the at least one sensor system; and
at least one principal game unit operably connected to the at least one signal detector, the at least one principal game unit including at least one signal transmitter configured to transmit a signal responsive to the at least one signal detector.

61. The game system of claim 60, wherein the at least one game component is configured to at least partially encircle part of the individual player's body.

62. The game system of claim 60, wherein the at least one game component is integral to the at least one principal game unit.

63.-64. (canceled)

65. The game system of claim 60, wherein the at least one game component comprises:
at least one flavorant.

66. (canceled)

67. The game system of claim 60, wherein the at least one game component comprises:
at least one reservoir.

68.-69. (canceled)

70. The game system of claim 60, wherein the at least one game component comprises:
at least one selective medium.

71. The game system of claim 60, wherein the at least one game component comprises:
an encapsulating material.

72. The game system of claim 60, wherein the at least one game component comprises:
at least one unique identifier.

73.-79. (canceled)

80. The game system of claim 60, wherein the at least one sensor system comprises:
at least one recognition element.

81.-89. (canceled)

90. The game system of claim 60, wherein the at least one signal detector is integral to the at least one principal game unit.

91. The game system of claim 60, wherein the at least one principal game unit comprises:
a port for communication with the at least one game component.

92.-93. (canceled)

94. The game system of claim 60, wherein the at least one principal game unit comprises:
at least one signal transmitter configured to transmit a signal to at least one external device.

95. The game system of claim 60, wherein the at least one principal game unit comprises:
at least one signal receiver configured to receive a signal from at least one external device.

96. The game system of claim 60, wherein the at least one principal game unit comprises:
at least one unique identifier.

97. The game system of claim 60, wherein the at least one principal game unit comprises:
at least one region configured to pair with the at least one game component.

98. (canceled)

99. The game system of claim 60, further comprising:
at least one physiological sensor.

100.-105. (canceled)

106. The at least one sensor system of claim 60, comprising:
an encapsulating material.

107. (canceled)

108. The game system of claim 60, comprising:
one or more display configured to be responsive to the at least one sensor system.

109. The game system of claim 60, comprising:
at least one antenna operably connected to the at least one principal game unit.

110. The game system of claim 60, comprising:
at least one antenna operably connected to the at least one sensor system.

111.-112. (canceled)

113. The game system of claim 60, further comprising:
at least one user interface device operably connected to the at least one principal game unit.

114. A method of determining the presence or absence of one or more analyte in at least one bodily fluid from an individual game player through a game interaction comprising:
assessing at least one bodily fluid from an individual game player for one or more analyte with at least one sensor integral to at least one component of a game system; and
indicating information from the assessment to at least one system user.

115.-117. (canceled)

118. The method of claim 114, wherein assessing at least one bodily fluid from an individual game player comprises:
binding the one or more analyte with a recognition element.

119.-126. (canceled)

127. The method of claim 114, wherein assessing at least one bodily fluid from an individual game player comprises:
assessing the at least one bodily fluid for the one or more analyte with at least one sensor integral to an individual game component.

128. The method of claim 114, wherein assessing at least one bodily fluid from an individual game player comprises:
assessing the at least one bodily fluid for the one or more analyte with at least one sensor integral to a principal game unit.

129.-135. (canceled)

136. The method of claim 114, comprising:
providing, with an individual game component, at least one substance to the individual game player; and
assessing for one or more metabolite of the at least one substance with the at least one sensor.

137.-139. (canceled)

140. The method of claim 114, comprising:
providing one or more flavorant with an individual game component.

141.-142. (canceled)

143. The method of claim 114, comprising:
receiving one or more unique identifier signals from a module associated with the individual game player.

144.-146. (canceled)
